(19) United States
(12) Patent Application Publication
(10) Pub. No.: US 2026/0007597 A1
(43) Pub. Date: Jan. 8, 2026

(54) COMPOSITIONS AND METHODS FOR TREATMENT OF VIRUS-INDUCED AIRWAY FIBROSIS

(71) Applicant: The United States of America, as represented by the Secretary, Dept. of Health and Human Services, Bethesda, MD (US)

(72) Inventors: Peter D. Sun, Rockville, MD (US); Rachel Erickson, Philadelphia, PA (US)

(73) Assignee: The United States of America, as represented by the Secretary, Dept. of Health and Human Services, Bethesda, MD (US)

(21) Appl. No.: 18/993,347

(22) PCT Filed: Jul. 10, 2023
(86) PCT No.: PCT/US2023/069899

§ 371 Date: Jan. 10, 2025

Related U.S. Application Data
(60) Provisional application No. 63/388,498, filed on Jul. 12, 2022.

(51) Int. Cl. A61K 9/00; A61K 31/245; A61K 31/403; A61K 31/427; A61K 31/4439; A61K 31/4709; A61K 31/7068; A61K 45/06; C07K 16/10

(52) U.S. Cl. CPC A61K 9/0078; A61K 31/245; A61K 31/403; A61K 31/427; A61K 31/4439; A61K 31/4709; A61K 31/7068; A61K 45/06; C07K 16/1003

(57) ABSTRACT

Methods of treating or inhibiting viral infection-induced airway fibrosis in a subject, the method including administering to the subject an effective amount of a composition including one or more direct thrombin inhibitors or one or more serine protease inhibitors or metalloprotease inhibitors are provided. In some examples, the composition is administered to the subject by inhalation. In particular examples, the viral infection-induced airway fibrosis is a coronavirus infection-induced airway fibrosis.

### CROSS REFERENCE TO RELATED APPLICATIONS

This application claims the benefit of U.S. Provisional Application No. 63/388,498, filed on Jul. 12, 2022, which is incorporated by reference in its entirety.

### FIELD

This disclosure relates to methods of treating lung fibrosis, particularly airway fibrosis related to viral infection.

### BACKGROUND

COVID-19 is an acute respiratory disease caused by the coronavirus SARS-COV-2. Early autopsy of COVID-19 patients revealed the presence of extensive pulmonary fibrosis, a likely cause of reduced oxygen intake and the need for ventilation in many hospitalized patients. Thrombotic structures were observed in pulmonary artery, vein, and microvasculature sites. However, despite the use of low molecular weight heparin as a standard thromboprophylaxis to mitigate coagulopathy, mortality persists. In addition to vascular thrombosis, SARS-COV-2 infected lungs often exhibit diffuse alveolar damage (DAD), as characterized by the presence of a proteinaceous intra-alveolar exudate that forms part of hyaline membranes. The development of DAD was also prominent in fatal H1N1 influenza, SARS-COV, and MERS infections. These fibrin-containing hyaline membranes may also promote development of pulmonary fibrosis. Due to continued prevalence of SARS-COV-2 infection worldwide, there remains a need for effective therapeutics.

### SUMMARY

Disclosed herein are methods of treating or inhibiting infection-induced (such as viral infection-induced) airway fibrosis. The methods include administering therapeutic agents, such as direct thrombin inhibitors or serine protease or metalloproteinase inhibitors to the airway.

Provided herein are methods of treating or inhibiting viral infection-induced airway fibrosis in a subject, the method including administering to the subject an effective amount of a composition including one or more direct thrombin inhibitors. In particular examples, the viral infection-induced airway fibrosis is a coronavirus infection-induced airway fibrosis (for example, infection-induced airway fibrosis caused by severe acute respiratory syndrome (SARS)-CoV-2 infection).

In some examples, the composition including one or more direct thrombin inhibitors is administered by inhalation, for example, as an aerosol. In some examples, the composition is administered using a nebulizer, a dry powder inhaler, or a metered dose inhaler. In some examples, the direct thrombin inhibitor includes one or more of hirudin, lepirudin, desirudin, bivalirudin, argatroban, dabigatran, and ximelagatran. In additional examples, the composition further includes a pharmaceutically acceptable carrier. In some specific examples, the direct thrombin inhibitor is argatroban or dabigatran. In further examples, the dose of argatroban or dabigatran administered to the subject is about 0.1 μg/kg to about 10 mg/kg.

Also provided herein are methods of treating or inhibiting viral infection-induced airway fibrosis in a subject, the method including administering to the subject an effective amount of a composition including one or more serine protease inhibitors or metalloprotease inhibitors. In particular examples, the viral infection-induced airway fibrosis is a coronavirus infection-induced airway fibrosis (for example, infection-induced airway fibrosis caused by SARS-COV-2 infection).

In some examples, the composition including the one or more serine protease inhibitors or metalloprotease inhibitors is administered by inhalation, for example, as an aerosol. In some examples, the composition is administered using a nebulizer, a dry powder inhaler, or a metered dose inhaler. In some examples, the serine protease inhibitor is camostat or nafamostat or the metalloprotease inhibitor is batimastat (BB-94) or prinomastat. In additional examples, the composition further includes a pharmaceutically acceptable carrier.

The disclosed methods may further include administering to the subject an additional treatment for the viral infection, such as one or more of an antiviral compound, a corticosteroid, and a monoclonal antibody. In some examples, the subject has a SARS-COV-2 infection and the antiviral compound is one or more of nirmatrelvir, ritonavir, remdesivir, and molnupiravir. In other examples, the subject has a SARS-COV-2 infection and the monoclonal antibody is bebtelovimab.

The foregoing and other features of the disclosure will become more apparent from the following detailed description, which proceeds with reference to the accompanying figures.

### BRIEF DESCRIPTION OF THE DRAWINGS

FIGS. 1A-1F shows proteomics analysis of COVID bronchoalveolar lavage fluid (BALF). FIG. 1A shows the number of proteins identified and overlaps among various BALF samples by mass spectrometry. H878, H902, and H906 are from healthy individuals, C3146 is from an acute COVID individual, and R3428 is from a recovered COVID individual. FIG. 1B shows that the average overlap among all five BALF samples decreases with identified protein abundance. Most abundant proteins exhibit greater than 80% overlap and are common to the five BALF samples, whereas low abundance proteins show less overlap and more unique to each sample. FIG. 1C shows Pearson correlation coefficient between pairwise samples calculated based on the abundances of 163 common proteins in all five samples. FIG. 1D is a heatmap showing differential protein abundance among mass spectrometry identified proteins in the five BALF samples. Plasma proteins, complement components, and coagulation factors (coag) were upregulated in acute COVID BALF sample. FIG. 1E is a heatmap displaying the fold change, measured as a ratio between the abundance of a protein in individual samples and its average abundance from all five samples, for each coagulation factor. n.d. stands for not detected in the BALF sample. FIG. 1F shows concentrations of total fibrinogen, prothrombin, and IgG present in healthy, acute COVID, and recovered COVID BALF samples as measured by ELISA.

FIG. 2 is a heatmap displaying differential abundance of mass spectrometry-identified BALF proteins involved in complement pathway. Samples labeled H878, H902, and H906 are BALF samples from healthy donors, C3146 and R3428 are from acute and recovered COVID individuals, respectively. Proteins not detected by mass spectrometry are labeled as n.d.

FIGS. 3A-3G show SARS-COV-2 pseudovirus infections and fibrin clot formation. FIG. 3A shows SARS-COV-2 pseudovirus infection of ACE2-293T and 293T cells (left panel) and NHBE cells (right panel). Cells were infected with 50 μl Wuhan strain of SARS-COV-2 pseudovirus, approximately 5×10⁶ copies of RNA/ml, for 48 hours. Cells were lysed and infections were measured by luciferase activity. FIGS. 3B and 3C show that infected NHBE cells induced fibrin clot formation. NHBE cells were grown in 96-well (FIG. 3B) or 384-well plate (FIG. 3C) to near confluence and infected with 5 μl (FIG. 3B) or titration amount (FIG. 3C) of Wuhan pSARS-2 for 24 hours before adding fibrinogen for clotting turbidity assay. Absorbance was taken at 350 nm with Synergy_h1 plate reader. FIG. 3D is confocal images of fibrin clot formation in NHBE cells infected with pSARS-2 (left) or uninfected cells (right) in the presence of fluorescently labelled fibrinogen. FIG. 3E is a SEM image showing of fibrin network observed in infected NHBE sample. FIG. 3F shows fibrin fibers associated with the infected (top) but not uninfected (bottom) NHBE cells. FIG. 3G shows that infected NHBE or human small airway epithelial cells (HSAEC) cells, but not Vero-E6 or ACE2-293T cells, induced fibrin clot formation. All cells were infected with equal amount (4 μl each) of delta strain SARS-COV-2 pseudovirus for 24 hours before adding fibrinogen for clotting turbidity assay. Data shows mean±SD. P values from unpaired t tests. ****P<0.0001.

FIGS. 4A-4H show SARS-COV-2 pseudovirus infection and fibrin clotting. FIG. 4A shows SARS-COV-2 pseudovirus infection in Vero E6 cells. Vero E6 cells were infected with SARS-CoV-2 pseudovirus for 24 hours. Cells were lysed and infection measured using luciferase assay. FIG. 4B shows SARS-COV-2 pseudovirus infection in NHBE cells. NHBE cells were infected with varying doses of SARS-COV-2 pseudovirus for 24 hours. Cells were lysed and infection measured using luciferase assay. FIG. 4C shows VSV pseudovirus infection in 293T cells. ACE2-expressing 293T or 293T cells were infected with VSV pseudovirus for 24 hours. Cells were lysed and infection measured using luciferase assay. FIG. 4D shows thrombin induced fibrin clotting. Thrombin was added to purified fibrinogen, and fibrin clot formation was measured by turbidity assay. OD was read with a plate reader at 350 nm. Data shows mean±SD. P values from unpaired t tests. ****P<0.0001. FIGS. 4E and 4F show confocal (FIG. 4E) and SEM (FIG. 4F) images of thrombin-induced fibrin clotting. Thrombin was added to fluorescently labelled fibrinogen. Fibrin clot formation was visualized with confocal microscopy. FIG. 4G shows viral dose dependent fibrin clotting results of FIG. 3C presented as area under the curve (AUC). FIG. 4H is SEM image of fibrin clots associated with SARS-COV-2 pseudovirus infected NHBE cells. Scale bar=20 μm.

FIGS. 5A-5D show fibrin clot formation from NHBE cells infected with different variants of SARS-COV-2. FIG. 5A shows NHBE cells that were grown in a 96-well plate and infected with 4 μl of different variant spike-typed pSARS-2 for 24 hours before adding fibrinogen for clotting turbidity assay. FIG. 5B shows fibrin clotting induced by replication competent SARS-COV-2 variants. FIG. 5C shows confocal images of fibrin clots observed in the presence of WA-1, beta, and delta variant infected NHBE cells. FIG. 5D shows SEM images of fibrin clots in the presence of SARSCOV-2 WA-1 or beta variant-infected NHBE cells. Data shows mean±SD. P values from unpaired t tests. ****P<0.0001.

FIGS. 6A and 6B show WA-1 strain of SARS-COV-2 infection of air-liquid interface cultured NHBE cells. FIG. 6A shows kinetics of viral titer expansion in infected NHBE cells. FIG. 6B shows an exemplary plaque assay used to determine the viral titer at each time point.

FIGS. 7A and 7B show inhibition of SARS-COV-2 infection-induced fibrin clot formation. FIG. 7A shows fibrin clot formation induced by Wuhan pSARS infected NHBE cells was suppressed by a serine protease inhibitor, camostat. FIG. 7B shows hirudin inhibited the fibrin clot formation by WA-1, beta, and delta strains of replication competent SARS-COV-2 infection of NHBE cells.

FIGS. 8A-8F show SARS-COV-2 induced fibrin clotting is thrombin dependent. FIG. 8A shows inhibition of thrombin (˜0.2 U/ml) induced fibrin clotting in the presence or absence of stoichiometric concentration of hirudin. FIG. 8B shows Wuhan SARS-COV-2 pseudovirus infected or uninfected NHBE cells assayed for fibrin clot formation in the presence of or absence of 5 U/ml hirudin, 5 μM dabigatran, or 5 μM argatroban. NHBE cells were infected for 24 h with SARS-CoV-2. Hirudin was added to infected and uninfected cells during fibrin clotting assay. Data shows mean±SD. P values from unpaired t tests. ****P<0.0001. FIG. 8C shows confocal images of fibrin clotting observed in pSARS infected and uninfected NHBE cells in the presence of hirudin, dabigatran and argatroban. Fluorescently labelled fibrinogen was added to cells 24 hours post infection. FIG. 8D shows fibrin clotting of WA-1 strain of SARSCOV-2 infected NHBE cells in the presence of titrating amount of hirudin. FIG. 8E shows confocal images of fibrin clotting observed in SARS-COV-2 delta variant infected NHBE cells in the absence (left), presence of 5 U/ml hirudin (middle), and in uninfected cells. FIG. 8F shows mass spectrometry identification of proteins in pSARS-2 infected NHBE cell culture supernatant. After 24 hour infection with Wuhan variant pSARS-2 virus, NHBE cell culture media was removed and cells washed with PBS once before incubating them with PBS for 1 hour to collect supernatants from both infected and uninfected NHBE cells for mass spectrometry analyses. Seven peptides were mapped to regions of thrombin catalytic domain, as indicated by short bars, from infected but not uninfected samples.

FIGS. 9A-9F show fibrin clot formation induced by NHBE cells and supernatant. FIG. 9A shows NHBE cells were infected with 5×10⁶ copies of RNA/ml of pSARS-2 variants for 24 hours. Cell culture supernatants (50 μl) were transferred to separate wells. Fibrinogen were added to both supernatants and cells for clotting assay. Data shows means±SD. P values from unpaired t tests. ****P<0.0001. FIG. 9B shows enzymatic cleavage of fluorescent Thrombin-324 peptide by Factor Xa and NHBE supernatant. Factor Xa recombinant protein or supernatant from infected/uninfected NHBE cells were added to thrombin-324 peptide and enzymatic activity was measured by increase in fluorescence over time. FIGS. 9C and 9D show expression of members of TMPRSS gene family, ST14 and TMPRSS11D, in various cells as measured by counts per 10 million total reads (TPM) from RNAseq (FIG. 9C), and western blot (FIG. 9D). FIG. 9E shows metalloproteinase inhibitors, BB-94 and prinomastat, but not others, inhibited infected NHBE cells induced fibrin clotting. The inhibitors were added during the viral infection, but not during fibrin clotting assay. FIG. 9F shows pSARS-2 infection resulted in the release of soluble ST14 in the culture supernatant.

FIG. 10 shows inhibition of the clotting step by Wuhan pSARS infected NHBE supernatants. As BB-94 reduced fibrin clot formation in FIG. 9E, further experiments were performed to clarify if the inhibition by BB-94 was on the infection or fibrin clotting steps. The fibrin clot formation was performed in the presence of various protease inhibitors. This experiment differed from that shown in FIG. 9E in that the inhibitors were added post-infection during the fibrin clotting assay, but not during the infection, whereas the inhibitors in FIG. 9E were included during the infection. Thus, BB-94, an inhibitor for ADAM metalloproteinases, reduced fibrin clot formation only if it was added during the infection but not during clotting, supporting the shedding of transmembrane serine proteases is important in the infection-induced fibrin clot formation.

FIGS. 11A-11C show contribution of matriptase and HAT in fibrin clot formation. FIG. 11A shows enzymatic cleavage of the prothrombin peptide, Thrb-324, by recombinant matriptase and HAT. FIG. 11B shows recombinant matriptase and HAT cleaved prothrombin for fibrin clot formation similar to factor Xa. FIG. 11C shows treatment with 25 ng ST14 (matriptase) or 50 ng TMPRSS11D (HAT) in transfected ACE2-293T cells induced fibrin clot formation. Infected (I) or uninfected (UI) ACE2-293T cells without transfection did not form fibrin clots.

FIGS. 12A-12C show SARS-COV-2 infection promoted fibrin clotting in COVID BALF. Delta variant pSARS-2 infected (pSARS-2) or uninfected (UI) NHBE cells were incubated with fibrinogen or various healthy (H877, H880, H882, H879, H883) (FIG. 12A), COVID-acute (C3263, C3267, C3146 and C3189) (FIG. 12B), and COVID-recovered (R3200, R3261, R3151, R3188, R3219, R3232 and R3248) (FIGS. 12B and 12C) BALF samples in fibrin clotting assays. The formation of fibrin clots was observed using confocal microscope with incorporation of sub-stoichiometric amount of fluorescent TAMRA labeled fibrinogen.

FIGS. 13A and 13B show concentration of fibrinogen (FIG. 13A) and prothrombin (FIG. 13B) in various healthy and COVID BALF samples as measured by ELISA.

FIGS. 14A and 14B are schematic diagrams illustrating a model for SARS-COV-2 infection-induced fibrosis. FIG. 14A illustrates that SARS-COV-2 viral infection directly activates prothrombin for fibrin clot formation. The viral-induced fibrin clotting does not require classical coagulation factors. FIG. 14B is a model for SARS-COV-2 infection induced lung fibrosis. 1) SARS-COV-2 infects lung cells. 2) Infected cells shed TTSPs from cell surface. 3) TTSPs cleave prothrombin into thrombin. 4) Thrombin cleaves fibrinogen into fibrin, which aggregates in the lung.

### SEQUENCE LISTING

Any nucleic acid and amino acid sequences listed herein and in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is the amino acid sequence of residues 324-333 of prothrombin: FNPRTFGSGE

### DETAILED DESCRIPTION

### I. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an inhibitor” includes singular or plural inhibitors and can be considered equivalent to the phrase “at least one inhibitor.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Aerosol: A suspension of fine solid particles or liquid droplets in a gas (such as air).

Administration: The introduction of a composition (such as a direct thrombin inhibitor or protease inhibitor) into a subject by a chosen route, such as via inhalation. In some examples herein, one or more direct thrombin inhibitors, one or more serine protease inhibitors, or one or more metalloprotease inhibitors are administered as an aerosol via inhalation (such as using a nebulizer).

Coronavirus: A family of positive-sense, single-stranded RNA viruses that are known to cause severe respiratory illness. Viruses currently known to infect humans from the coronavirus family are from the alphacoronavirus and betacoronavirus genera. Additionally, it is believed that the gammacoronavirus and deltacoronavirus genera may potentially infect humans in the future.

Non-limiting examples of betacoronaviruses include SARS-COV-2, Middle East respiratory syndrome coronavirus (MERS-COV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and transmissible gastroenteritis coronavirus (TGEV). A non-limiting example of a deltacoronavirus is the swine delta coronavirus (SDCV).

The viral genome is capped, polyadenylated, and covered with nucleocapsid proteins. The coronavirus virion includes a viral envelope containing type I fusion glycoproteins referred to as the spike(S) protein. Most coronaviruses have a common genome organization with the replicase gene included in the 5′-two thirds of the genome, and structural genes included in the 3′-third of the genome.

Direct thrombin inhibitor: A compound that directly inhibits thrombin activity, for example, by binding to thrombin and blocking its interaction with substrates and/or its activity. This is as compared to indirect thrombin inhibitors, which block generation and activity of thrombin upstream in the thrombosis process. Direct thrombin inhibitors include bivalent inhibitors (such as hirudin, bivalirudin, lepirudin, and desirudin), which bind to the active site and exosite 1 of thrombin, acting as competitive inhibitors. Univalent inhibitors (such as argatroban, inogatran, melagatran (and its prodrug ximelagatran), and dabigatran) block the active site of thrombin. Direct thrombin inhibitors also include allosteric inhibitors (such as DNA aptamers, benzofuran dimers or trimers, and polymeric lignins (such as sulfated β-O4 lignin)).

Fibrosis: A condition associated with the thickening and scarring of connective tissue. Often, fibrosis occurs in response to an injury, such as from a disease or condition that damages tissue. Fibrosis is an exaggerated wound healing response that when severe, can interfere with normal organ function. Fibrosis can occur in almost any tissue of the body, including in the lungs or airway. In some examples, fibrosis of the lung or airway is induced by viral infection, such as infection with a coronavirus (such as SARS-COV-2). In some examples, diffuse alveolar damage (DAD), characterized by presence of fibrin-containing hyaline membranes, may precede fibrosis.

Influenza virus: Influenza viruses are enveloped negative-strand RNA viruses belonging to the orthomyxoviridae family. Influenza viruses are classified on the basis of their core proteins into three distinct types: A, B, and C. Within these broad classifications, subtypes are further divided based on the characterization of two antigenic surface proteins, hemagglutinin (HA or H) and neuraminidase (NA or N). While B and C type influenza viruses are largely restricted to humans, influenza A viruses are pathogens of a wide variety of species including humans, non-human mammals, and birds. Periodically, non-human strains, particularly of swine and avian influenza, have infected human populations, in some cases causing severe disease with high mortality. Reassortment between such swine or avian strains and human strains in co-infected individuals has given rise to reassortant influenza viruses to which immunity is lacking in the human population, resulting in influenza pandemics. Four such pandemics occurred during the past century (pandemics of 1918, 1957, 1968, and 2009) and resulted in numerous deaths world-wide.

Influenza viruses have a segmented single-stranded (negative or antisense) genome. The influenza virion consists of an internal ribonucleoprotein core containing the single-stranded RNA genome and an outer lipoprotein envelope lined by a matrix protein. The segmented genome of influenza consists of eight linear RNA molecules that encode ten polypeptides. Two of the polypeptides, HA and NA, include the primary antigenic determinants or epitopes required for a protective immune response against influenza. Based on the antigenic characteristics of the HA and NA proteins, influenza strains are classified into subtypes. For example, recent outbreaks of avian influenza in Asia have been categorized as H1N1, H5N1, H7N3, H7N9, and H9N2 based on their HA and NA phenotypes.

HA is a surface glycoprotein which projects from the lipoprotein envelope and mediates attachment to and entry into cells. The HA protein is approximately 566 amino acids in length, and is encoded by an approximately 1780 base polynucleotide sequence of segment 4 of the genome. In addition to the HA antigen, which is the predominant target of neutralizing antibodies against influenza, the neuraminidase (NA) envelope glycoprotein is also a target of the protective immune response against influenza. NA is an approximately 450 amino acid protein encoded by an approximately 1410 nucleotide sequence of influenza genome segment 6. Recent pathogenic avian strains of influenza have belonged to the N1, N2, N3, and N9 subtypes.

Metalloprotease inhibitor: An agent that inhibits activity of a metalloprotease. In some examples, the metalloprotease inhibitor is an inhibitor of one or more ADAM (a disintegrin and metalloproteinase) metalloproteases, for example, is a compound that decreases or inhibits activity of an ADAM. In some examples, the ADAM inhibitor is BB-94 (batimastat), which has the structure:

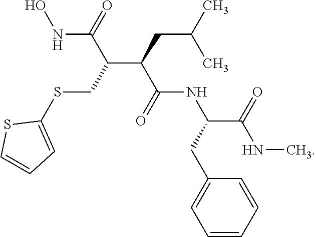 [SMILES: CNC(=O)[C@H](Cc1ccccc1)NC(=O)[C@H](CC(C)C)[C@H](CSc1cccs1)C(=O)NO]

In other examples, the ADAM inhibitor is prinomastat, which has the structure:

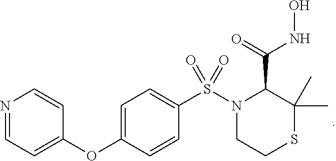 [SMILES: CC1(C)SCCN(S(=O)(=O)c2ccc(Oc3ccncc3)cc2)[C@H]1C(=O)NO]

Microparticles: Solid colloidal particles that range in size from about 0.1 to 100 microns. They can be made from biodegradable and biocompatible biomaterials. Active components, such as drugs, can be adsorbed, encapsulated, or covalently attached to their surface or into their matrix.

Nanoparticles: Solid colloidal particles that range in size from about 10-1000 nm. They can be made from biodegradable and biocompatible biomaterials. Active components, such as drugs, can be adsorbed, encapsulated, or covalently attached to their surface or into their matrix.

Nebulizer: A device for converting a therapeutic agent in liquid form into a mist or fine spray (an aerosol) that can be inhaled into the respiratory system, such as the lungs. A nebulizer is also known as an “atomizer.” Exemplary nebulizers include AEROECLIPSE® II Breath Actuated Nebulizer (BAN), AirLife Sidestream nebulizer, or AEROGEN® Ultra vibrating mesh nebulizer.

Pharmaceutically acceptable carriers: Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of the compositions herein disclosed. In general, the nature of the carrier will depend on the particular mode of administration being employed. For instance, liquid formulations usually comprise fluids that include pharmaceutically and physiologically acceptable fluids such as water, physiological saline, balanced salt solutions, aqueous dextrose, glycerol or the like as a vehicle. For solid compositions (e.g., powder forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition (such as fibrosis) after it has begun to develop. “Ameliorating” refers to the reduction in the number or severity of signs or symptoms of a disease.

SARS-COV-2: Also known as Wuhan coronavirus or 2019 novel coronavirus, SARS-COV-2 is a positive-sense, single stranded RNA virus of the genus betacoronavirus that has emerged as a highly fatal cause of severe acute respiratory infection. The viral genome is capped, polyadenylated, and covered with nucleocapsid proteins. The SARS-COV-2 virion includes a viral envelope with large spike glycoproteins. The SARS-COV-2 genome, like most coronaviruses, has a common genome organization, with the replicase gene included in the 5′-two thirds of the genome, and structural genes included in the 3′-third of the genome. The SARS-COV-2 genome encodes the canonical set of structural protein genes in the order 5′-spike (S)-envelope (E)-membrane (M)-nucleocapsid (N)-3′. Symptoms of SARS-COV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-COV-2 infection, including but not limited to, assessment of patient symptoms and background and genetic tests such as reverse transcription-polymerase chain reaction (rRT-PCR) or antigen-based tests. The test can be done on patient samples such as nasal swab, respiratory (such as BALF), or blood samples.

Serine protease inhibitor: An agent that inhibits or decreases activity of a serine protease. In particular examples, a serine protease inhibitor is a type II transmembrane serine protease (TTSP) inhibitor, for example, is a compound that decreases or inhibits activity of a TTSP. In particular examples, the TTSP is matriptase or human airway trypsin-like protease (HAT). An exemplary TTSP inhibitor is camostat, which has the structure:

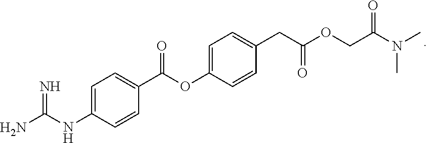 [SMILES: CN(C)C(=O)COC(=O)Cc1ccc(OC(=O)c2ccc(NC(=N)N)cc2)cc1]

Another exemplary TTSP inhibitor is nafamostat, which has the structure:

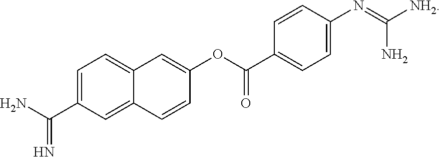 [SMILES: N=C(N)c1ccc2cc(OC(=O)c3ccc(N=C(N)N)cc3)ccc2c1]

Subject: Living multi-cellular vertebrate organisms, a category that includes both human and veterinary subjects, including human and non-human mammals. In some examples, the subject has lung fibrosis, such as virus-induced airway fibrosis.

Therapeutically effective amount: A quantity of a specified agent (such as a direct thrombin inhibitor or protease inhibitor) sufficient to achieve a desired effect in a subject, cell, or sample being treated with that agent. In some examples, the therapeutically effective amount is the amount of an agent (such as a direct thrombin inhibitor or protease inhibitor) sufficient to decrease fibrin clot formation, either in vitro or in vivo. For example, the agent or agents can decrease the size or number of fibrin clots by a desired amount, for example by at least 5%, at least 10%, at least 15%, at least 20%, at least 25%, at least 30%, at least 50%, at least 75%, at least 90%, or at least 95% as compared to a response in the absence of the agent. In other examples, the therapeutically effective amount is the amount of a direct thrombin inhibitor or protease inhibitor sufficient to treat or inhibit lung fibrosis (such as virus-induced lung fibrosis) in a subject.

### II. Overview

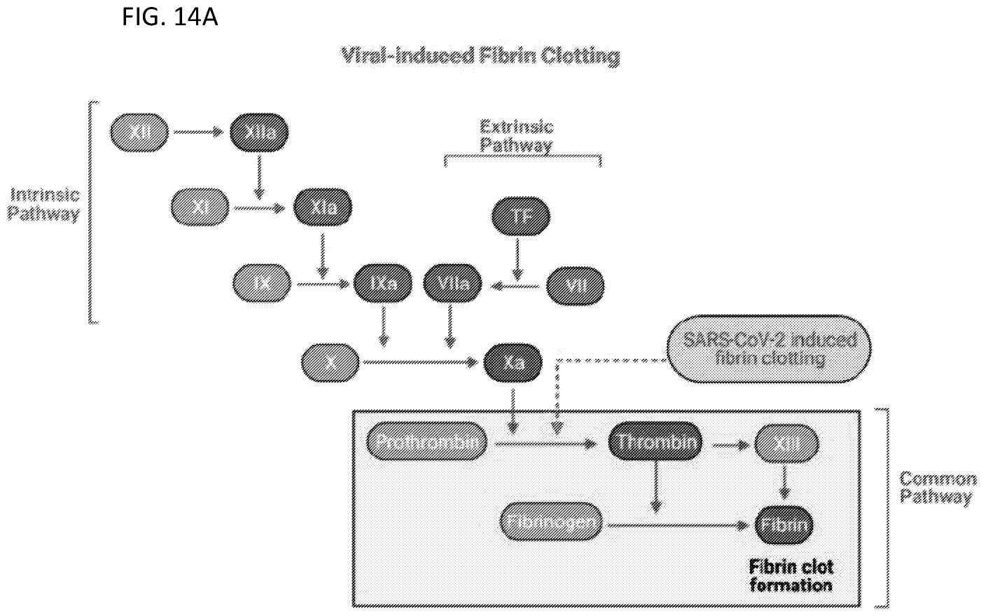

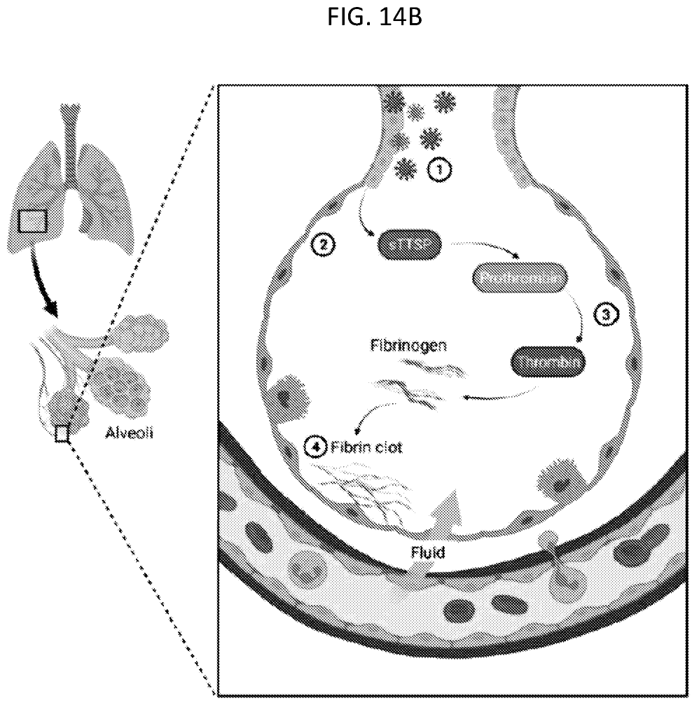

The classical coagulation pathway refers to a sequential activation of a network of serine proteases leading to thrombin-mediated fibrin clotting or thrombosis, a critical process to prevent excessive bleeding in wound healing. Dysregulated thrombosis, such as venous thromboembolism (VTE), is known to contribute to morbidity and mortality in cancer patients. For COVID-associated lung fibrosis, various mechanisms, including TGF-β mediated extracellular collagen fiber formation and neutrophil extracellular traps, have been proposed. One proposed mechanism attributes lung fibrosis to the inflammatory activation of the classical extrinsic coagulation pathway and its leakage through blood lining endothelial cells to infected lung. This is further exacerbated by increased tissue factor expression found in infected NHBE cells. However, the findings disclosed herein support a cell-mediated thrombosis that occurs in alveolar airway space independent of plasma coagulations (FIG. 14A). SARS-COV-2 infection-induced release of activated transmembrane serine proteases, such as matriptase and HAT, by infected lung epithelial cells activates prothrombin (FIG. 14B).

As described herein, the concentration of prothrombin and fibrinogen in BALF varied considerably between healthy and COVID individuals. The highest concentrations were found in acute COVID samples and decreased to healthy levels in recovered COVID samples. Consistently, the healthy and most of the recovered COVID BALF did not form fibrin clots in the presence of infected NHBE cells. In contrast, fibrin clot formations were observed in 3 of 4 acute COVID BALF in the presence of SARS-COV-2 infection, showing a significant risk of fibrin clots in acute COVID lung fluids. The direct contribution of the viral infection to fibrin clotting was evident as minimal or no clotting was detected in acute COVID BALF in the absence of the viral infection. However, fibrinogen concentration is not the only deciding factor for fibrin clotting in BALF and there are likely other fibrinolytic factors that influence the infection-induced fibrin clot formation. The clinical risk of developing pulmonary fibrosis due to COVID has not been well characterized, although preexisting pulmonary conditions, severity of infection, and the presence of inflammatory factors appear to predict the risk of COVID associated lung fibrosis.

The current use of heparin family of anticoagulants, while beneficial, have not mitigated COVID associated lung fibrosis (Becker, J. Thromb. Thrombolysis 50:54-67, 2020). Heparin related compounds target primarily activated clotting factor Xa with partial inhibition of thrombin activity. Intravenous or subcutaneous injection of low molecular weight heparin has been used to prevent microvascular thrombosis in hospitalized COVID patients. As described herein, a SARS-CoV-2 infected NHBE cell-triggered fibrin clotting mechanism occurs in the alveolar space outside of blood circulation and is independent of coagulation factor Xa. Thus, administration of heparin targeting factor Xa intravenously may be less effective. Instead, a more effective therapeutic intervention focused on using inhaled (such as nebulized) direct thrombin inhibitors or serine protease or metalloprotease inhibitors to target airway space is provided.

### III. Methods of Treatment

Provided herein are methods of treating or inhibiting infection-induced airway fibrosis (such as formation of fibrin clots in the lung) induced by a viral infection. The methods include administering to a subject a direct thrombin inhibitor or a serine protease inhibitor or metalloprotease inhibitor by inhalation.

The subject may have any viral infection that causes infection-induced airway fibrosis, particularly formation of fibrin clots in the lung or diffuse alveolar damage (DAD). In some examples, the subject is infected with or suspected to be infected with a coronavirus, including, but not limited to SARS-COV, SARS-COV-2, or MERS. In other examples, the subject is infected with or suspected to be infected with an influenza virus. However, any viral infection that causes or increases airway fibrosis or fibrin clot formation in the lung may be present.

In some examples, the disclosed methods can decrease the size or number of fibrin clots by a desired amount, for example by at least 5%, at least 10%, at least 15%, at least 20%, at least 25%, at least 30%, at least 50%, at least 75%, at least 90%, or at least 95% as compared to a response in the absence of treatment or as compared to prior to treatment. In some examples, fibrin clot formation is measured in vitro using BALF samples from the subject (for example, before and after treatment). Exemplary methods for such assays are provided in Examples 1 and 2, below.

In some examples, the subject is administered a direct thrombin inhibitor via inhalation. Exemplary direct thrombin inhibitors include hirudin, lepirudin, desirudin, bivalirudin, argatroban, dabigatran, inogatran, and melagatron or its prodrug ximelagatran. In particular examples, the direct thrombin inhibitor is argatroban or dabigatran. In some examples, the direct thrombin inhibitor or a pharmaceutically acceptable salt thereof is formulated for administration by inhalation.

In other examples, the subject is administered a protease inhibitor, such as a serine protease inhibitor or a metalloprotease inhibitor by inhalation. In some examples, the serine protease inhibitor is an inhibitor of a type II transmembrane serine protease (TTSP). TTSPs share a common structure including a cytoplasmic N-terminal domain, a transmembrane domain and an extracellular C-terminal serine protease domain. In some examples, the TTSP inhibitor decreases or inhibits activity of one or more of matriptase (ST14) and TMPRSS11D (HAT). An exemplary TTSP inhibitor is camostat (such as camostat mesylate). Another exemplary TTSP inhibitor is nafamostat. Additional TTSP inhibitors can be selected. See, e.g., Murza et al. (Expert Opinion on Therapeutic Patents, 30:807-824, 2020). In some examples, the TTSP inhibitor or a pharmaceutically acceptable salt thereof is formulated for administration by inhalation.

In other examples, the metalloprotease inhibitor is an inhibitor of an ADAM metalloprotease. ADAMs are a unique family of cell membrane-associated calcium-dependent zinc-containing matrix metalloproteases and they are believed responsible for shedding of cell-surface membrane-associated receptors, such as TTSP (or TMPRSS) receptors. In some examples, the ADAM inhibitor decreases or inhibits activity of one or more of ADAM10 or ADAM17. Exemplary ADAM inhibitors include BB-94 (batimastat) and prinomastat. In some examples, the ADAM inhibitor or a pharmaceutically acceptable salt thereof is formulated for administration by inhalation.

In some examples, a pharmaceutically acceptable salt of the direct thrombin inhibitor or protease inhibitor may be administered to the subject. Pharmaceutically acceptable salts of a compound described herein include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Description of suitable pharmaceutically acceptable salts can be found in Handbook of Pharmaceutical Salts, Properties, Selection and Use, Wiley V C H (2002).

Pharmaceutically acceptable acid addition salts are a subset of pharmaceutically acceptable salts that retain the biological effectiveness of the free bases while formed by acid partners. In particular, the compound may form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, and the like.

Pharmaceutically acceptable base addition salts are a subset of pharmaceutically acceptable salts that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine.

The direct thrombin inhibitor or serine protease (such as TTSP) inhibitor or metalloprotease (such as an MMP, for example, an ADAM) inhibitor can be administered to humans or other animals in various manners. In particular examples, the disclosed agents are administered to the airway or lungs, for example by inhalation. By way of example, one method of administration to the airway or lungs is by inhalation through the use of a nebulizer or inhaler. For example, a composition including the direct thrombin inhibitor or serine protease (such as TTSP) inhibitor or metalloprotease (such as ADAM) inhibitor is formulated in an aerosol or particulate and drawn into the lungs using a nebulizer. In some examples, the composition is administered using a nebulizer. Any nebulizer capable of converting the composition into an aerosol with an appropriate droplet size for delivery to the lung can be used. In some examples, the nebulizer is an AEROECLIPSE® II Breath Actuated Nebulizer (BAN), an AirLife Sidestream nebulizer or an AEROGEN® Ultra vibrating mesh nebulizer. In other examples, the composition is administered using a dry powder inhaler or a metered dose inhaler.

The compositions or pharmaceutical compositions can include a nanoparticle or microparticle including a direct thrombin inhibitor or serine protease (such as TTSP) inhibitor or metalloprotease (such as MMP, for example, ADAM) inhibitor, which can be administered locally, such as by pulmonary inhalation or intra-tracheal delivery. When nanoparticles are provided, or microparticles including or consisting of these nanoparticles are provided, e.g. for inhalation, they are generally suspended in an aqueous carrier, for example, in an isotonic buffer solution at a pH of about 3.0 to about 8.0, preferably at a pH of about 3.5 to about 7.4, 3.5 to 6.0, or 3.5 to about 5.0. Useful buffers include sodium citrate-citric acid and sodium phosphate-phosphoric acid, and sodium acetate-acetic acid buffers.

For administration by inhalation, nanoparticles or microparticles including the direct thrombin inhibitor or serine protease (such as TTSP) inhibitor or metalloprotease (such as MMP or ADAM) inhibitor, or compositions including the direct thrombin inhibitor or serine protease (such as TTSP) inhibitor or metalloprotease (such as MMP or ADAM) inhibitor can be conveniently delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges for use in an inhaler or insufflator can be formulated containing a powder mix of the compound and a suitable powder base such as lactose or starch.

The site of particle deposition within the respiratory tract is generally demarcated based on particle size. In one example, particles of about 10 to about 500 microns are utilized, such as particles of about 25 to about 250 microns, or about 10 to about 25 microns are utilized. In other examples, particles of about 0.5 to 50 microns are utilized. For use in a metered dose inhaler for administration to lungs, particles of less than about 10 microns, such as particles of about 2 to about 8 microns, such as about 0.5 to about 5 microns, such as particles of about 0.5 to about 2 microns, can be utilized. In general, the goal for particle size for inhalation is about 1-2 μm or less in order that the composition reaches the alveolar region of the lung for absorption. Actual methods of preparing such dosage forms are known, or will be apparent, to those of ordinary skill in the art.

In some examples, the subject is administered argatroban or dabigatran and the therapeutically effective amount of argatroban or dabigatran administered by inhalation can be from about 0.1 μg/kg to about 1 mg/kg body weight. In other examples, a therapeutically effective amount of argatroban or dabigatran can be from about 1 mg/kg to about 10 mg/kg of body weight. In some examples, a therapeutically effective amount of argatroban or dabigatran can be from about 0.1 μg/kg to about 10 mg/kg of body weight (such as about 0.1 μg/kg to about 1 μg/kg, about 0.5 μg/kg to about 5 μg/kg, about 2.5 μg/kg to about 10 μg/kg, about 7.5 μg/kg to about 20 μg/kg, about 15 μg/kg to about 50 μg/kg, about 25 μg/kg to about 75 μg/kg, about 50 μg/kg to about 100 μg/kg, about 100 μg/kg to about 250 μg/kg, about 200 μg/kg to about 500 μg/kg, about 500 μg/kg to about 1 mg/kg, about 750 μg/kg to about 2.5 mg/kg, about 2 mg/kg to about 5 mg/kg, about 3 mg/kg to about 7.5 mg/kg, or about 6 mg/kg to about 10 mg/kg of body weight). A skilled clinician can select appropriate doses for administration via inhalation to a subject, based on preclinical and clinical trials, the particular therapeutic agent utilized, the type and severity of infection, the condition of the subject, and other factors.

The particular mode of administration and the dosage regimen will be selected by the attending clinician, taking into account the particulars of the case (e.g. the subject, the disease, the disease state involved, and the condition of the subject). In cases in which more than one agent or composition is being administered, one or more routes of administration may be used; for example, a direct thrombin inhibitor or serine protease or metalloprotease inhibitor may be administered by inhalation and an additional therapy for the viral infection may be administered orally or intravenously. Treatment can involve daily or multi-daily doses of compound(s) over a period of a few days to weeks, months, or more. In other examples, the treatment involves administering the compound(s) every other day, twice weekly, weekly, every other week, or monthly.

In some examples, the subject is infected with or is suspected to be infected with a coronavirus, such as SARS-COV-2. In other examples, the subject is infected with or is suspected to be infected with an influenza virus. In some examples, the subject is hospitalized and receiving supplemental oxygen. In other examples, the subject is hospitalized and on a ventilator.

In some examples, the subject is treated with one or more additional therapies for the viral infection. The additional treatment may include, but is not limited to, one or more of an antiviral compound, a corticosteroid, and a monoclonal antibody. In particular examples, the subject has a coronavirus infection (such as SARS-COV-2) and is further treated with one or more antiviral compounds, such as nirmatrelvir, ritonavir, remdesivir, and/or molnupiravir. In other particular examples, the subject has a coronavirus infection (such as SARS-COV-2) and is further treated with a monoclonal antibody, such as bebtelovimab.

### EXAMPLES

The following examples are provided to illustrate certain particular features and/or examples. These examples should not be construed to limit the disclosure to the particular features or examples described.

### Example 1

### Materials and Methods

Cells and viruses: Normal Human Primary Bronchial/Tracheal Epithelial (NHBE) cells, Vero E6 cells, and HEK 293T cells were purchased from American Type Culture Collection (ATCC, Manassas, VA) and cultured according to the manufacturer's guidance. In particular, NHBE cells (ATCC, catalog PCS-300-010) were cultured in Airway Epithelial Cell Basal Media (ATCC, PCS-300-030) supplemented with Bronchial/Tracheal Epithelial Cell Growth Kit (ATCC, PCS-300-040) under standard tissue culture conditions (37° C. and 5% CO₂). NHBE cells were harvested by washing with Dulbecco's phosphate-buffered saline (DPBS) (ATCC, 30-2200), then incubated with trypsin-EDTA (Life Technologies Corp, NY) at 37° C. for 5 min. The cells were resuspended in Airway Epithelial media for continued passage or cryopreservation. Cell counts were performed using a Guava Muse Cell Analyzer according to manufacturer's protocol (Luminex, TX). ACE2-expressing 293T cells (Catalog SL221) were purchased from Genecopoeia, Rockville MD. The culturing of NHBE cells in air-liquid interface was performed according to the manufacturer's instructions (Stemcell Technologies). Briefly, 3.3×10⁴ or 4.5×10⁵ cells in 0.2 or 3 mL PneumaCult™-Ex Plus Medium were plated in each transwell insert of 24- or 6-well plates (Corning, 3413 or 3450) with 0.5 or 3 mL respectively, of the same medium added into the basal chamber. After 2-3 days when confluence was reached, the medium from both the basal and apical chambers was removed and 0.5 or 3 mL respectively, of PneumaCult™-ALI Maintenance Medium (Stemcell Technologies, 05001) was added to the basal chamber and cells were cultured for 28 days with media change every 1-2 days. Beginning in week 2 post-airlift, mucus was removed from the apical surface by washing the cells with D-PBS.

Circulating variants of SARS-COV-2 viruses were expanded and characterized as described previously (Liu et al., Proc. Natl. Acad. Sci. USA 118: e2109744118, 2021). B.1.1.7 (alpha variant) and Washington-1 isolates were provided by BEI resources (Manassas, VA), B.1.351 (beta) and B.1.617.2+AY.1+AY.2 (delta) variants were kind gifts from Dr. Andrew Pekosz of Johns Hopkins University, Baltimore, MD.

Bronchoalveolar lavage fluid (BALF) from healthy donors was purchased from Audubon Biosciences with informed consent (New Orleans, LA). BALF from COVID-experienced donors was collected at Indiana University through a CLIA approved clinical BAL laboratory. All samples were obtained for clinical indications in patients with acute and post-COVID lung disease. All samples were deidentified before analyses. For fibrin clotting assays, BALF samples were dialyzed against 0.045% of NaCl solution over night to remove excess salts and then concentrated 20-fold using a Speedvac (Labconco CentriVap) concentrator with the heating turned off.

Production of SARS-COV-2 pseudoviruses: For the production of the pseudovirus, HEK 293 T cells were plated at a density of 2.5×10⁶ per 10 cm plate and incubated at 37°C./5% CO₂ overnight. Cells were co-transfected with a SARS-COV-2 spike protein plasmid and an HIV NL4-3 env-nef-luciferase core using Lipofectamine 3000 according to the manufacturers protocol. Plasmids encoding SARS-COV-2 spike genes, including Wuhan, alpha (B.1.1.7), beta (B.1.351), gamma (Brazil strain), delta (B.1.617.2), and omicron (B.1.1.529) strains were obtained from Addgene. Supernatant containing pseudovirus particles was harvested 48 hours post transfection and concentrated 100-fold using PEG-it Virus Precipitation Solution (System Biosciences, CA). The concentration of SARS-COV-2 pseudovirus was estimated by RT-PCR in numbers of RNA copies/ml. In brief, RNA was extracted from 50 μl concentrated pseudovirus using the Qiagen RNeasy Mini Kit, and cDNA was generated using a C1000 Touch Thermal cycler (BIO-RAD, CA 94547) with ABI High-Capacity cDNA Reverse Transcription Kit following the manufacturer's protocol. HIV-1 NL4-3 LTR was amplified using TaqMan HIV-1 LTR primer/probe sets (Pa03453409_s1) from ThermoFisher with 50 ng cDNA as template. Samples were run in duplicate using a QuantStudio 6 Pro Real-Time PCR System (ThermoFisher, MA) together with a serial dilution of a known copy number HIV DNA as standards. The pseudovirus concentrations were between 10⁸-10⁹ copies of RNA/ml. The infectivity of SARS-COV-2 pseudovirus was examined by a luciferase assay, in which ACE2 expressing 293T cells or NHBE cells were plated in 96-well plates and grown to near confluence. The cells were infected with titration volume of the pseudoviruses between 5×10⁶-5×10⁵ copies of RNA/ml in their growth media. Polybrene was added at 5 μg/ml concentration to NHBE cells. Luciferase activity was assayed after 48 hours of infection using Luc-Pair firefly luciferase HS assay kit according to the manufacturer's protocol (Genecopoeia, Inc), and luminescence was measured by Synergy_h1 plate reader (BioTek, Inc). The Washington, UK, and South Africa strains of SARS-COV-2 viruses were expanded by infecting TMPRSS2-expressing Vero-E6 cells.

Infection of NHBE cells with SARS-COV-2 pseudoviruses: For infection-induced fibrin clotting assay, NHBE cells growing at 60-80% confluence were infected with SARS-COV-2pseudovirus at doses between 0.05-4 μl virus per 10,000 cells, or between 40-1 copies of viral RNA per cell, for 24 hours prior to clotting assays. The infected supernatant was then removed and replaced with fibrinogen containing clotting buffer.

For transfection of TMPRSS genes, ACE2-expressing HEK 293T cells (Genecopoeia, Inc. MD) were plated at a density of 40,000 cells per well in a 96 well plate and incubated at 37° C., 5% CO2 in DMEM growth media supplemented with 10% FBS for overnight. Plasmids encoding ST14 (OHu19145C) or TMPRSS11D (OHu04628C) were synthesized in pcDNA3.1 vector with eGFP attached to N-terminus of the genes (GenScript). Cells were transfected with either ST14 or TMPRSS11D plasmids using Lipofectamine 3000 according to the manufacturer's protocol. Transfected cells were cultured with fresh media for 48 hours and infected with titration amount of pseudovirus in cell culture media. After overnight infection, the cell culture supernatants were used in the fibrin clotting assay.

Fibrin clotting turbidity assay: Purified fibrinogen from human plasma (Sigma-Aldrich, MO) was dissolved in 100 mM NaCl, 20 mM HEPES buffer. The solution was incubated at 37° C. for 10 minutes, then filtered through a 0.45 μm syringe filter. The solution was stored at 4° C. for 30 minutes, then filtered again to remove aggregates. Concentration was measured using nanodrop, then the solution was aliquoted and frozen at −20° C.

Clot formation was assayed using fibrinogen solution diluted to 1.5 μM concentration in clotting buffer (20 mM HEPES, 137 mM NaCl, 5 mM CaCl₂). Diluted fibrinogen was added to thrombin enzyme (5 U/mL, Sigma) (positive control) or infected/uninfected NHBE cells seeded in a 96 well plate at 10,000 cells/well, or in a 384 well plate at 2500 cells/well for overnight. The absorbance was measured at 350 nm wavelength continuously with 2 min intervals for 4-10 hours with Synergy_H1 (BioTek) plate reader. Fibrin clot formation causes scattering of light that passes through the solution, which increases the turbidity. For component-based fibrin clotting assays, 100 ng of human prothrombin (Millipore, catalog 539515) was incubated with 100 ng factor Xa (R&D systems, Inc. catalog 1063-SE-010) or 500 ng recombinant matriptase (R&D systems, Inc. catalog 3946-SEB-010) or 200 ng of HAT (R&D systems, Inc. catalog 2695-SE-010) in 40 μl volume in a 384-well plate at room temperature for one hour in 20 mM HEPES, 137 mM NaCl, 5 mM CaCl2 prior to adding 1.5 μM fibrinogen to the mix. Upon addition of fibrinogen, the fibrin clotting was monitored with absorbance at 350 nm every 2 min on a plate reader.

SEM Sample Preparation: Clotting assays were performed in a 24-well plate with 5×7 mm silicon chips (Ted Pella Inc., CA) immersed. Upon clotting, samples were fixed with 2% paraformaldehyde, and then post-fixed with 1.0% osmium tetroxide/0.8% potassium ferricyanide in 0.1 M sodium cacodylate buffer, stained with 1% tannic acid in dH₂O. After additional buffer washes, the samples were further osmicated with 2% osmium tetroxide in 0.1 M sodium cacodylate, then washed with dH₂O. Specimens were dehydrated with a graded ethanol series, critical point dried under CO₂ in a Bal-Tec model CPD 030 Drier (Balzers, Liechtenstein), mounted on aluminum studs, and sputter coated with 35 Å of iridium in a Quorum EMS300T D sputter coater (Electron Microscopy Sciences, Hatfield, PA) prior to viewing at 5 kV in a Hitachi SU-8000 field emission scanning electron microscope (Hitachi, Tokyo, Japan).

Enzymatic cleavage of prothrombin: Fluorogenic peptide substrate corresponding to residues 324-333 of prothrombin gene, referred to as Thrb-324, was synthesized as dabcyl-FNPRTFGSGE-edans (SEQ ID NO: 1) by Biomatik. The peptide encompasses the factor Xa cleavage site. The cleavage of fluorogenic Thrb-324 peptide was initiated by mixing 10 μM fluorogenic peptide with 100 ng of human factor Xa (R & D systems, Inc), or 400 ng of human matriptase (R & D Systems, Inc) in 100 μl assay buffer containing 25 mM Tris at pH 9.0, 2.5 μM ZnCl₂, and 0.005% Brij-35 (w/v), or with infected cells or 100 μl of infected supernatant in 96-well plates. The cleavage of Thrb-324 peptide was detected using a Synergy_H1 fluorescent plate reader (BioTek) with 340 nm excitation and 490 nm emission wavelengths for 3 hours at 37° C.

Western Blot: NHBE cells were plated in 6-well plates and incubated at 37° C., 5% CO₂ for 24 hours. Cells were infected with SARS-COV-2 pseudovirus for 24 hours. Following infection, media was removed from cells and cells were washed with DPBS twice. Media was replaced with 50 mM HEPES, 250 mM NaCl buffer. Cells in buffer were incubated at 37° C., 5% CO₂ for 0.5-1 hour, then cells and supernatant were harvested. The cells were lysed with RIPA lysis buffer containing protease inhibitors. Proteins in supernatant were precipitated with 20% trichloroacetic acid (TCA) at 4° C. at least 10 min. The precipitated protein was spun down at 18,000 g for 5 min, then washed two times with 200 μl cold acetone. The pellet was dried and then dissolved in SDS buffer for gel electrophoresis using NuPAGE 4-12% Bis-Tris gel. For western blot, proteins were transferred from the gel to PVDF membranes using iBlot transfer apparatus. The membrane was blocked with PBS containing Tween and 2% BSA for 5 minutes at RT, then incubated with primary antibody (Anti ST14: A6135 from Abclonal, anti-TMPRSS11D: PA5-87660 from Invitrogen) for 1 hr at RT or 4° C. overnight. After three 5-minute washes with blocking buffer, appropriate secondary antibodies were added for 1 hr at RT. Membrane was developed using SuperSignal West Dura Extended Duration Substrate (Thermo).

Imaging of fibrin fibers by confocal microscopy: Fibrinogen was labeled with a fluorescent dye TAMRA-SE (Thermo Fisher Scientific, catalog c1171) according to the manufacturer's protocol. The fluorescent TAMRA-fibrinogen was added to fibrin clotting assays at 80 g/ml concentration or mixed with unlabeled fibrinogen at 1:6 ratio. Images were taken on a Zeiss LSM 880 confocal microscope equipped with Plan-Apochromat 20×/0.8 M27 objective. Z-stacks were performed to image fibrin formation. After acquisition, maximum intensity projections of the z-stacks were made using Fiji.

Proteomics analyses by mass spectrometry: Twenty microliter aliquots of BALF samples were dissolved in SDS-sample buffer and applied onto a 4-12% Nupage gel with MOPS running buffer. The run stopped after the samples migrated approximately ¼ distance into the gel. Each lane of the gel was sliced into smaller pieces, and subjected to destaining, reducing/alkylation, and in-gel trypsin digestion. Peptides were extracted using a 2 cm Pepmap 100 C18 trap column and a 25 cm Easy-spray Pepmap 100 C18 analytical column. The extracted peptides from the gel fractions were applied for LC-MS/MS analysis using either a Thermo Orbitrap Fusion or a Thermo Orbitrap Fusion Lumos operated with an in-line Thermo nLC 1200 and an EASY-Spray ion source. Both instrument acquisitions were operated at a 120,000 resolution (m/z 200) with a scan range of 350-1950 m/z and CID fragmentation. All data were processed using Proteome Discoverer v2.4 (Thermo Scientific) with a SEQUEST HT search against the Uniprot KB/Swiss-Prot Human Proteome (02/2021) and common contaminants (theGPM.org) using a 5 ppm precursor mass tolerance and a 0.5 Da fragment tolerance. Dynamic modifications included in the search were limited to oxidation [M], deamidation [NQ], and acetylation [Protein N-terminal] while carbamidomethylation [C] was the only static modification utilized. Peptides and proteins were filtered at a 1% FDR using a target-decoy approach with a two peptide per protein minimum. Relative protein abundance was estimated from an average of its top three unique peptide intensities as determined by chromatographic area-under-the-curve and normalized by total intensity of all peptides. Pearson correlation coefficients between samples were calculated using normalized abundance of each protein with exclusion of serum albumin and immunoglobulin genes, whose abundances are donor dependent. The differential abundance is calculated as percentage of difference in abundance: by dividing the difference abundance between a protein in one sample and the average abundance of the protein with the average abundance of the protein from all healthy samples. The list of proteins used for the differential abundance heatmap analysis includes the ones with average healthy abundance greater than 25 and all non-zero abundance in the acute COVID sample. The heatmaps display the fold change in abundance relative to the average of each protein.

Fibrinogen, prothrombin and IgG ELISA: ELISA assays were used to determine the levels of fibrinogen (Abcam, ab108841), total IgG (Abcam, ab195215), and prothrombin (Molecular Innovations, HPTKT-TOT) present in human BALF samples. The samples were diluted with kit specific assay diluents. For prothrombin and fibrinogen levels, samples were evaluated at 1:50 and 1:500 dilutions. For the total IgG ELISA, samples were evaluated at 1:1,000 and 1:10,000 dilutions. The assays were carried out following the manufacturer's protocols.

RNAseq sample preparation: Total RNA was extracted from approximately 1×10⁶ NHBE or HSAEC cells with Trizol (Invitrogen, Carlsbad, CA, USA) according to manufacturer's instructions. Ten μg of purified RNA from each sample was sent to Genewiz commercial sequencing facility (South Plainfield, NJ) for Bioanalyzer quality control analysis (Agilent, Santa Clara, CA) and Illumina Next Generation Sequencing. All the submitted total RNA samples had an RNA integrity number (RIN) of 10.

### Example 2

### Elevated Prothrombin and Fibrinogen Levels in COVID Lung Fluid

COVID-19 associated lung fibrosis was previously thought to be the result of dysregulated coagulation leading to thrombosis in veins as evidenced from frequent microthrombi formation in diseased lungs. Further, plasma D-dimer levels appeared to correlate with the severity and mortality of COVID-19. However, despite the use of anti-coagulants such as heparin in hospitalized COVID patients, the clinical onset of COVID-associated lung fibrosis continued to drive mortality. In addition to microvascular thrombosis, hyaline membrane formation, a hallmark of acute respiratory distress syndrome (ARDS), was also frequently observed in COVID lungs, suggesting the presence of inflammatory exudate containing plasma-borne coagulation factors in infected alveolar space. Indeed, activated monocytes and macrophages as well as inflammatory cytokines were detected in cells from bronchoalveolar lavage (BAL). However, how COVID affects coagulation components in SARS-COV-2 infected bronchoalveolar lavage fluid (BALF) has remained unclear.

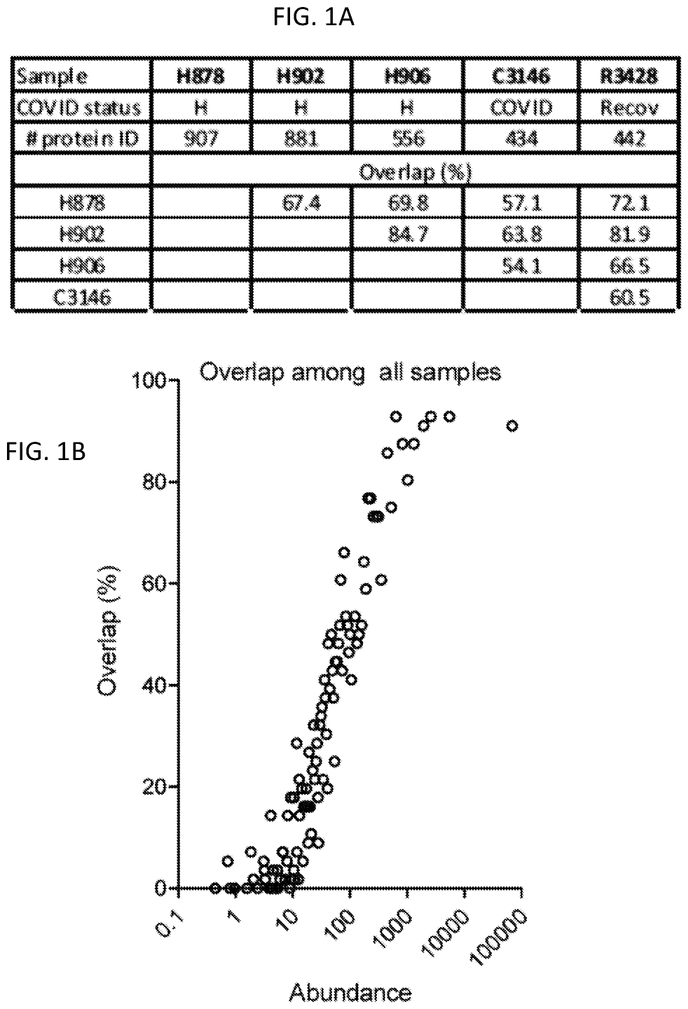

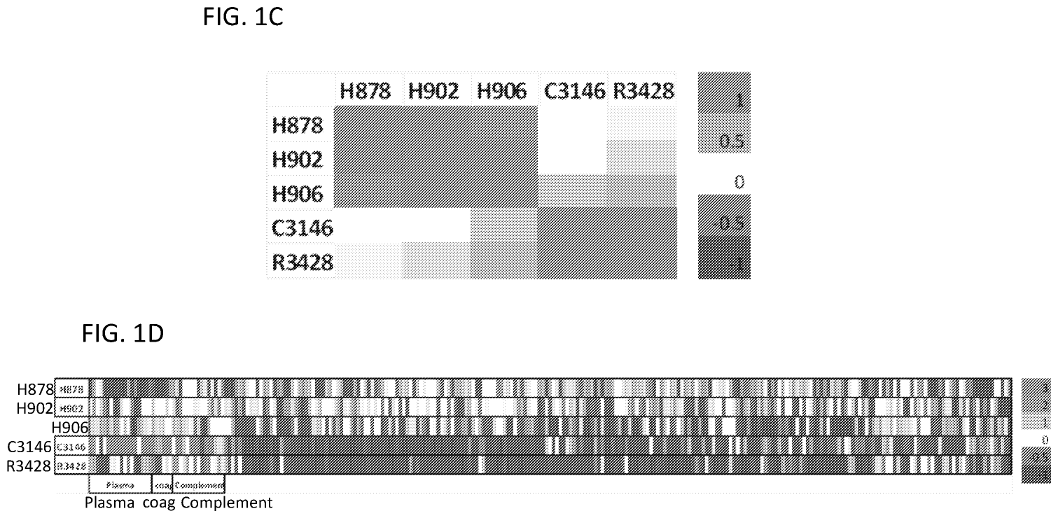

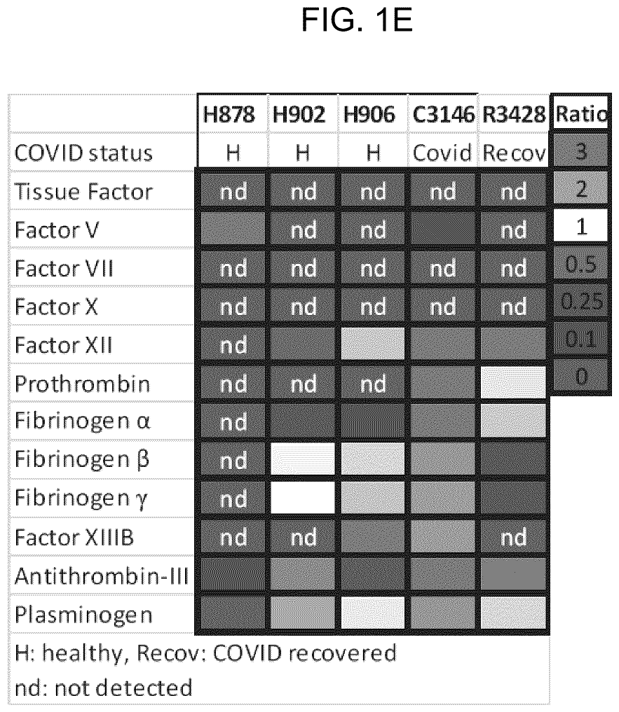

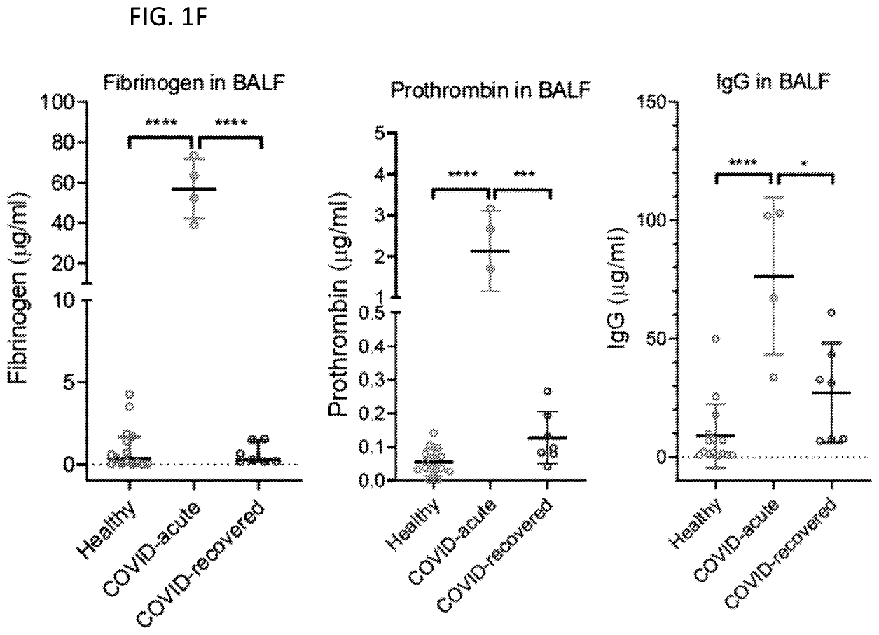

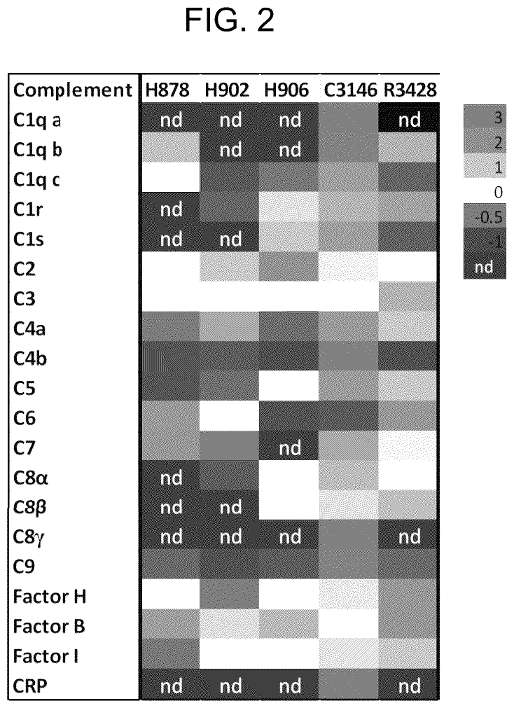

To address SARS-COV-2 infection-induced changes in protein contents in COVID lungs, mass spectrometry-based proteomics analysis on BALF from three healthy donors, one acute (COVID-acute) donor, and one recovered (COVID-recovered) donor was performed. The acute and recovered COVID samples were taken on the day of or more than 30 days after discharge from hospital, respectively. Overall, the mass spectrometry proteomic analyses identified between 400 and 900 proteins from each BALF sample with 55-80% overlap (common proteins) between samples (FIG. 1A, Table 1). The overlaps in identified proteins correlated with their abundance, with the most abundant proteins showing greater than 90% overlap (FIG. 1B), suggesting similar compositions of enriched proteins in healthy, COVID-acute, and COVID-recovered lungs. When the covariance in protein abundance was compared using a Pearson correlation coefficient analysis among a subset of 163 proteins common to all five samples, it showed that protein abundances were more correlated among healthy as well as between the COVID samples but less correlated between healthy and COVID samples (FIG. 1C), suggesting SARS-COV-2 infection resulted in systematic changes in protein enrichment in lungs. While both healthy and COVID-experienced BALF samples contained many enriched plasma proteins, immunoglobulins, complement factors and SERPIN family of protease inhibitors (Table 1), the abundance of proteins in several classes differed systematically between the samples. There was a clear increase of enriched plasma proteins in the acute COVID sample compared to the healthy ones (FIG. 1D), suggesting an elevated infiltration of plasma into the infected lung. The presence of inflammatory response in the COVID-acute sample was evident from the presence of C-reactive protein and an overall enrichment in complement components in the acute COVID compared to the healthy samples (FIG. 2). Several coagulation factors, including prothrombin, fibrinogen, FXII, FXIIIB, antithrombin III and plasminogen were identified by mass spectrometry (FIG. 1E, Table 1), and most of them showed enhanced abundance in the acute COVID sample compared to the healthy samples (FIG. 1E).

TABLE 1
Abundance of proteins in healthy and COVID BALF

| | | Protein Abundance in Samples | | | | |
|---|---|---|---|---|---|---|
| Accession | Description | H878 | H902 | H906 | C3146 | C3428 |
| | Pulmonary Proteins | | | | | |
| Q8IWL2 | Pulmonary surfactant- | 0 | 0 | 0 | 29.12 | 28.95 |
| | associated protein A1 | | | | | |
| Q8IWL1 | Pulmonary surfactant- | 0 | 0 | 0 | 29.12 | 28.95 |
| | associated protein A2 | | | | | |
| P07988 | Pulmonary surfactant- | 485.73 | 60.63 | 0 | 214.45 | 53.21 |
| | associated protein B | | | | | |
| P35247 | Pulmonary surfactant- | 0 | 0 | 0 | 39.4 | 211.84 |
| | associated protein D | | | | | |
| P15941 | Mucin-1 | 1210.73 | 465.4 | 0 | 41.67 | 237.33 |
| Q99102 | Mucin-4 | 686.72 | 185.73 | 32.28 | 0 | 306.87 |
| P98088 | Mucin-5AC | 8159.27 | 7706.85 | 4512.9 | 6.01 | 104.93 |
| Q9HC84 | Mucin-5B | 80635.5 | 9158.56 | 5915.82 | 0 | 532.08 |
| Q8WXI7 | Mucin-16 | 2175.01 | 584.95 | 97.32 | 0 | 33.66 |
| | Common Plasma Proteins | | | | | |
| P02768 | Serum albumin | 54793.8 | 197688. | 447365. | 761895 | 551873. |
| P02787 | Serotransferrin | 7728.57 | 6938.3 | 21877.7 | 33125.8 | 30192.1 |
| P00450 | Ceruloplasmin | 5120.48 | 3650.61 | 3217.89 | 5021.18 | 1348.75 |
| P02788 | Lactotransferrin | 166535. | 79416.8 | 65437.0 | 7.55 | 3118.21 |
| P00738 | Haptoglobin | 605.36 | 4419.16 | 19228.8 | 10024.9 | 1328.95 |
| P69905 | Hemoglobin subunit alpha | 0 | 10.55 | 338.47 | 0 | 17.22 |
| P68871 | Hemoglobin subunit beta | 287.13 | 0 | 978.12 | 0 | 34.15 |
| P02790 | Hemopexin | 524.01 | 3778.71 | 6749.73 | 18829.4 | 5493.99 |
| P02763 | Alpha-1-acid glycoprotein 1 | 0 | 1103.72 | 817.23 | 5056.05 | 34.89 |
| P19652 | Alpha-1-acid glycoprotein 2 | 0 | 90.09 | 0 | 97.11 | 40.83 |
| P04217 | Alpha-1B-glycoprotein | 0 | 358.66 | 749.53 | 3120.81 | 477.63 |
| P02765 | Alpha-2-HS-glycoprotein | 98.58 | 157.98 | 193.27 | 2824.42 | 1031.98 |
| P01023 | Alpha-2-macroglobulin | 287.13 | 446.19 | 3531.83 | 11227.9 | 1343.8 |
| A8K2U0 | Alpha-2-macroglobulin-like | 145.72 | 3.37 | 19.72 | 0 | 0 |
| | protein 1 | | | | | |
| P02647 | Apolipoprotein A-I | 263.2 | 0 | 0 | 474.22 | 12.67 |
| P04114 | Apolipoprotein B-100 | 0 | 21.56 | 178.55 | 92.58 | 16.06 |
| P02649 | Apolipoprotein E | 0 | 0 | 5.78 | 27.9 | 0 |
| P02749 | Beta-2-glycoprotein 1 | 0 | 207.51 | 350.24 | 664.26 | 125.97 |
| P02751 | Fibronectin | 112.7 | 232.7 | 347.3 | 2057.29 | 556.82 |
| | Coagulation factors | | | | | |
| P02671 | Fibrinogen alpha chain | 32.3 | 166.31 | 56.12 | 2179.33 | 465.26 |
| P02675 | Fibrinogen beta chain | 0 | 1748.45 | 2128.91 | 3173.11 | 366.27 |
| P02679 | Fibrinogen gamma chain | 113.42 | 2156.21 | 3492.59 | 4480.71 | 569.2 |
| P00734 | Prothrombin | 0 | 0 | 0 | 430.64 | 93.79 |
| P12259 | Coagulation factor V | 74.41 | 0 | 0 | 1.8 | 0 |
| P00748 | Coagulation factor XII | 0 | 53.16 | 92.51 | 371.36 | 62.36 |
| P05160 | Coagulation factor XIII B | 0 | 0 | 28.84 | 20.57 | 0 |
| | chain | | | | | |
| P01008 | Antithrombin-III | 66.04 | 437.65 | 234.47 | 2528.03 | 405.86 |
| P00747 | Plasminogen | 70.59 | 123.18 | 161.88 | 385.31 | 150.22 |
| | Serine Protease Inhibitors | | | | | |
| | (SERPIN) | | | | | |
| P29622 | Serpin A4/Kallistatin | 0 | 51.66 | 38.65 | 97.46 | 65.09 |
| P05154 | Serpin A5/Plasma serine | 0 | 37.15 | 0 | 12.22 | 0 |
| | protease inhibitor | | | | | |
| P08185 | Serpin A6/Corticosteroid- | 0 | 0 | 0 | 217.93 | 0 |
| | binding globulin | | | | | |
| P05543 | Serpin A7/Corticosteroid- | 0 | 0 | 54.15 | 948.45 | 86.86 |
| | binding globulin | | | | | |
| Q9UK55 | Serpin A10/Protein Z- | 0 | 0 | 0 | 6.47 | 0 |
| | dependent protease inhibitor | | | | | |
| Q8IW75 | Serpin A12 | 0 | 0 | 78.19 | 0 | 0 |
| P30740 | Serpin B1/Leukocyte | 887.71 | 5379.85 | 4140.09 | 39.93 | 259.85 |
| | elastase inhibitor | | | | | |
| P05120 | Serpin B2/Plasminogen | 0 | 0 | 19.43 | 0 | 0 |
| | activator inhibitor 2 | | | | | |
| P29508 | Serpin B3 | 1698.85 | 486.75 | 347.3 | 0 | 124.73 |
| P48594 | Serpin B4 | 0 | 53.59 | 0 | 0 | 0 |
| P35237 | Serpin B6 | 375.66 | 307.42 | 144.22 | 0 | 43.31 |
| O75635 | Serpin B7 | 0 | 5.85 | 20.7 | 0 | 0 |
| P50452 | Serpin B8 | 0 | 41.42 | 10.4 | 0 | 0 |
| P50453 | Serpin B9 | 0 | 48.67 | 0 | 3.26 | 0 |
| P48595 | Serpin B10 | 0 | 452.59 | 79.76 | 0 | 0 |
| Q96P63 | Serpin B12 | 378.05 | 45.69 | 282.55 | 0 | 7.5 |
| Q9UIV8 | Serpin B13 | 0 | 0 | 20.11 | 0 | 0 |
| P01008 | Serpin C1/Antithrombin-III | 66.04 | 437.65 | 234.47 | 2528.03 | 405.86 |
| P05546 | Serpin D1/Heparin cofactor | 0 | 62.34 | 247.23 | 256.29 | 147.5 |
| | 2 | | | | | |
| P36955 | Serpin F1/Pigment | 9044.58 | 789.9 | 22.17 | 296.39 | 218.52 |
| | epithelium-derived factor | | | | | |
| P08697 | Serpin F2/Alpha-2- | 0 | 0 | 0 | 507.35 | 60.38 |
| | antiplasmin | | | | | |
| P05155 | Serpin G1/Plasma protease | 545.55 | 160.33 | 68.67 | 1917.81 | 267.28 |
| | C1 inhibitor | | | | | |
| | Complement Factors | | | | | |
| P02746 | Complement C1q | 60.78 | 0 | 0 | 319.05 | 56.92 |
| | subcomponent subunit B | | | | | |
| P02747 | Complement C1q | 235.92 | 52.52 | 92.12 | 512.58 | 74.24 |
| | subcomponent subunit C | | | | | |
| P00736 | Complement C1r | 0 | 21.99 | 94.28 | 128.32 | 34.15 |
| | subcomponent | | | | | |
| P09871 | Complement C1s | 0 | 0 | 70.54 | 93.62 | 12.7 |
| | subcomponent | | | | | |
| P06681 | Complement C2 | 189.51 | 142.61 | 111.84 | 296.39 | 249.95 |
| P01024 | Complement C3 | 7991.77 | 7472.02 | 5680.37 | 6642.61 | 4182.36 |
| P0C0L4 | Complement C4-A | 57.43 | 73.65 | 51.11 | 317.31 | 84.14 |
| P0C0L5 | Complement C4-B | 83.75 | 134.28 | 41.2 | 1743.47 | 45.78 |
| P01031 | Complement C5 | 32.3 | 75.36 | 156.97 | 451.56 | 120.03 |
| P13671 | Complement component C6 | 777.64 | 343.71 | 49.84 | 81.25 | 164.08 |
| P10643 | Complement component C7 | 57.43 | 49.96 | 0 | 244.09 | 143.78 |
| P07357 | Complement component C8 | 0 | 17.46 | 57.88 | 115.24 | 75.73 |
| | alpha chain | | | | | |
| P07358 | Complement component C8 | 0 | 0 | 66.61 | 93.97 | 119.04 |
| | beta chain | | | | | |
| P02748 | Complement component C9 | 92.84 | 34.8 | 68.97 | 829.89 | 92.31 |
| P08174 | Complement decay- | 85.42 | 127.24 | 0 | 9.94 | 38.61 |
| | accelerating factor | | | | | |
| P00751 | Complement factor B | 1088.7 | 3159.6 | 1226.33 | 2562.9 | 1041.88 |
| P08603 | Complement factor H | 1318.4 | 478.21 | 891.79 | 1523.79 | 551.87 |
| Q03591 | Complement factor H- | 0 | 100.55 | 79.27 | 116.81 | 90.33 |
| | related protein 1 | | | | | |
| P05156 | Complement factor I | 93.32 | 222.03 | 202.1 | 322.54 | 142.05 |
| P02741 | C-reactive protein | 0 | 0 | 0 | 106 | 0 |
| | Immunoglobulins | | | | | |
| P01876 | Immunoglobulin heavy | 90445.8 | 115496 | 51505.9 | 2405.98 | 67808.6 |
| | constant alpha 1 | | | | | |
| P01877 | Immunoglobulin heavy | 6101.5 | 6383.24 | 3767.29 | 124.13 | 1987.24 |
| | constant alpha 2 | | | | | |
| P01880 | Immunoglobulin heavy | 0 | 286.07 | 0 | 0 | 95.53 |
| | constant delta | | | | | |
| P01857 | Immunoglobulin heavy | 8302.83 | 65967.2 | 76228.7 | 36612.8 | 193527 |
| | constant gamma 1 | | | | | |
| P01859 | Immunoglobulin heavy | 624.51 | 5593.34 | 15402.7 | 5875.48 | 8414.22 |
| | constant gamma 2 | | | | | |
| P01860 | Immunoglobulin heavy | 308.66 | 4611.3 | 15206.5 | 1091.41 | 11903.6 |
| | constant gamma 3 | | | | | |
| P01861 | Immunoglobulin heavy | 180.17 | 1093.05 | 1059.55 | 209.22 | 2474.77 |
| | constant gamma 4 | | | | | |
| P01871 | Immunoglobulin heavy | 6915.04 | 6831.56 | 6857.64 | 301.62 | 1616.02 |
| | constant mu | | | | | |
| A0A0C4DH31 | Immunoglobulin heavy | 0 | 67.89 | 106.94 | 83.51 | 88.6 |
| | variable 1-18 | | | | | |
| P23083 | Immunoglobulin heavy | 0 | 0 | 275.68 | 0 | 299.45 |
| | variable 1-2 | | | | | |
| A0A0C4DH29 | Immunoglobulin heavy | 43.79 | 0 | 75.74 | 5.58 | 95.77 |
| | variable 1-3 | | | | | |
| A0A0A0MS14 | Immunoglobulin heavy | 0 | 273.26 | 0 | 0 | 83.65 |
| | variable 1-45 | | | | | |
| P01743 | Immunoglobulin heavy | 207.69 | 444.05 | 177.57 | 12.05 | 24.75 |
| | variable 1-46 | | | | | |
| P01742 | Immunoglobulin heavy | 0 | 0 | 31.1 | 1.44 | 59.89 |
| | variable 1-69 | | | | | |
| A0A0B4J2H0 | Immunoglobulin heavy | 0 | 0 | 26.98 | 1.44 | 0 |
| | variable 1-69D | | | | | |
| P0DP01 | Immunoglobulin heavy | 0 | 58.07 | 156.97 | 0 | 48.26 |
| | variable 1-8 | | | | | |
| A0A0B4J1V2 | Immunoglobulin heavy | 0 | 181.25 | 122.63 | 0 | 217.28 |
| | variable 2-26 | | | | | |
| A0A0C4DH43 | Immunoglobulin heavy | 0 | 0 | 0 | 252.8 | 306.87 |
| | variable 2-70D | | | | | |
| P01766 | Immunoglobulin heavy | 45.7 | 108.66 | 0 | 26.33 | 141.31 |
| | variable 3-13 | | | | | |
| A0A0B4J1V0 | Immunoglobulin heavy | 144.28 | 82.62 | 294.32 | 7.43 | 157.64 |
| | variable 3-15 | | | | | |
| A0A0C4DH32 | Immunoglobulin heavy | 0 | 26.69 | 22.86 | 1.57 | 0 |
| | variable 3-20 | | | | | |
| P01764 | Immunoglobulin heavy | 1205.94 | 0 | 26.68 | 652.06 | 21.36 |
| | variable 3-23 | | | | | |
| P01768 | Immunoglobulin heavy | 1205.94 | 757.88 | 82.7 | 0 | 57.41 |
| | variable 3-30 | | | | | |
| A0A0B4J1X8 | Immunoglobulin heavy | 77.76 | 198.54 | 330.62 | 0 | 134.13 |
| | variable 3-43 | | | | | |
| P0DP04 | Immunoglobulin heavy | 1306.44 | 459 | 887.86 | 144.71 | 0 |
| | variable 3-43D | | | | | |
| A0A0A0MS15 | Immunoglobulin heavy | 122.27 | 454.73 | 920.24 | 32.95 | 863.69 |
| | variable 3-49 | | | | | |
| P01767 | Immunoglobulin heavy | 0 | 0 | 0 | 5.82 | 42.07 |
| | variable 3-53 | | | | | |
| A0A0J9YX35 | Immunoglobulin heavy | 138.06 | 0 | 664.18 | 25.8 | 368.74 |
| | variable 3-64D | | | | | |
| A0A0C4DH42 | Immunoglobulin heavy | 0 | 0 | 0 | 5.82 | 42.07 |
| | variable 3-66 | | | | | |
| P01780 | Immunoglobulin heavy | 713.04 | 1878.68 | 2903.95 | 217.93 | 2442.6 |
| | variable 3-7 | | | | | |
| A0A0B4J1Y9 | Immunoglobulin heavy | 63.41 | 184.67 | 137.35 | 128.32 | 462.78 |
| | variable 3-72 | | | | | |
| A0A0B4J1V6 | Immunoglobulin heavy | 0 | 0 | 708.33 | 11.04 | 25.74 |
| | variable 3-73 | | | | | |
| A0A0B4J1X5 | Immunoglobulin heavy | 0 | 1895.76 | 68.48 | 200.5 | 876.07 |
| | variable 3-74 | | | | | |
| P01782 | Immunoglobulin heavy | 1306.44 | 459 | 887.86 | 144.71 | 1190.36 |
| | variable 3-9 | | | | | |
| A0A0C4DH34 | Immunoglobulin heavy | 0 | 0 | 459.14 | 0 | 908.24 |
| | variable 4-28 | | | | | |
| A0A087WSY4 | Immunoglobulin heavy | 0 | 268.99 | 0 | 0 | 366.27 |
| | variable 4-30-2 | | | | | |
| P06331 | Immunoglobulin heavy | 1086.31 | 0 | 88.3 | 0 | 93.79 |
| | variable 4-34 | | | | | |
| P0DP08 | Immunoglobulin heavy | 1086.31 | 57.43 | 45.33 | 0 | 5.84 |
| | variable 4-38-2 | | | | | |
| A0A0J9YXX1 | Immunoglobulin heavy | 320.63 | 0 | 0 | 259.78 | 0 |
| | variable 5-10-1 | | | | | |
| A0A0C4DH38 | Immunoglobulin heavy | 101.93 | 234.83 | 687.73 | 57.19 | 697.89 |
| | variable 5-51 | | | | | |
| A0A0B4J1U7 | Immunoglobulin heavy | 0 | 3757.36 | 0 | 0 | 999.81 |
| | variable 6-1 | | | | | |
| P01591 | Immunoglobulin J chain | 294.31 | 2369.7 | 254.1 | 0 | 171.5 |
| P01834 | Immunoglobulin kappa | 22491.82 | 52090.62 | 26586.86 | 19003.79 | 40586.23 |
| | constant | | | | | |
| P01599 | Immunoglobulin kappa | 0 | 108.66 | 96.83 | 18.48 | 0 |
| | variable 1-17 | | | | | |
| P01602 | Immunoglobulin kappa | 0 | 292.48 | 0 | 205.73 | 415.76 |
| | variable 1-5 | | | | | |
| A0A0C4DH72 | Immunoglobulin kappa | 0 | 39.49 | 82.41 | 0 | 76.47 |
| | variable 1-6 | | | | | |
| A0A0C4DH67 | Immunoglobulin kappa | 0 | 0 | 193.27 | 179.58 | 0 |
| | variable 1-8 | | | | | |
| P04432 | Immunoglobulin kappa | 0 | 0 | 0 | 273.72 | 26.97 |
| | variable 1D-39 | | | | | |
| A0A075B6P5 | Immunoglobulin kappa | 77.05 | 0 | 0 | 2.89 | 0 |
| | variable 2-28 | | | | | |
| P06310 | Immunoglobulin kappa | 51.44 | 0 | 56.31 | 12.55 | 4.5 |
| | variable 2-30 | | | | | |
| A0A075B6S6 | Immunoglobulin kappa | 0 | 0 | 0 | 12.55 | 4.5 |
| | variable 2D-30 | | | | | |
| P04433 | Immunoglobulin kappa | 223.48 | 71.94 | 1236.14 | 249.32 | 245 |
| | variable 3-11 | | | | | |
| P01624 | Immunoglobulin kappa | 1411.72 | 397.08 | 41.01 | 0 | 4999.04 |
| | variable 3-15 | | | | | |
| P01619 | Immunoglobulin kappa | 813.53 | 2689.93 | 680.86 | 659.03 | 113.84 |
| | variable 3-20 | | | | | |
| A0A0A0MRZ8 | Immunoglobulin kappa | 223.48 | 71.94 | 1236.14 | 249.32 | 245 |
| | variable 3D-11 | | | | | |
| A0A087WSY6 | Immunoglobulin kappa | 289.52 | 497.42 | 192.29 | 49.34 | 584.05 |
| | variable 3D-15 | | | | | |
| A0A0C4DH25 | Immunoglobulin kappa | 289.52 | 386.41 | 0 | 31.38 | 447.93 |
| | variable 3D-20 | | | | | |
| P06312 | Immunoglobulin kappa | 593.4 | 1492.27 | 1098.79 | 324.28 | 1309.15 |
| | variable 4-1 | | | | | |
| P0DOY2 | Immunoglobulin lambda | 3517.34 | 0 | 5405.67 | 1293.65 | 0 |
| | constant 2 | | | | | |
| P0DOY3 | Immunoglobulin lambda | 3517.34 | 65.75 | 5405.67 | 1293.65 | 292.02 |
| | constant 3 | | | | | |
| P01703 | Immunoglobulin lambda | 0 | 0 | 0 | 75.32 | 39.84 |
| | variable 1-40 | | | | | |
| P01701 | Immunoglobulin lambda | 0 | 0 | 0 | 29.99 | 34.15 |
| | variable 1-51 | | | | | |
| A0A075B6K4 | Immunoglobulin lambda | 0 | 0 | 179.53 | 37.31 | 0 |
| | variable 3-10 | | | | | |
| P01717 | Immunoglobulin lambda | 0 | 188.51 | 0 | 31.56 | 158.14 |
| | variable 3-25 | | | | | |
| A0A075B6K5 | Immunoglobulin lambda | 0 | 1485.86 | 226.63 | 45.33 | 187.34 |
| | variable 3-9 | | | | | |
| A0A075B6I0 | Immunoglobulin lambda | 0 | 0 | 110.86 | 27.72 | 9.5 |
| | variable 8-61 | | | | | |
| B9A064 | Immunoglobulin lambda- | 1684.49 | 851.81 | 2246.64 | 2074.73 | 2944.98 |
| | like polypeptide 5 | | | | | |
| | Other Proteins | | | | | |
| Q04446 | 1,4-alpha-glucan-branching | 51.92 | 33.94 | 63.77 | 0 | 0 |
| | enzyme | | | | | |
| P31946 | 14-3-3 protein beta/alpha | 49.53 | 61.06 | 0 | 0 | 0 |
| P62258 | 14-3-3 protein epsilon | 49.29 | 66.61 | 15.6 | 26.5 | 0 |
| P63104 | 14-3-3 protein zeta/delta | 88.05 | 68.96 | 24.82 | 20.4 | 0 |
| P16885 | 1-phosphatidylinositol 4,5- | 0 | 6.06 | 31.69 | 0 | 2.77 |
| | bisphosphate | | | | | |
| | phosphodiesterase gamma-2 | | | | | |
| P09543 | 2′,3′-cyclic-nucleotide 3′- | 16.73 | 29.89 | 0 | 0 | 0 |
| | phosphodiesterase | | | | | |
| Q99460 | 26S proteasome non-ATPase | 56.71 | 0 | 0 | 1.87 | 0 |
| | regulatory subunit 1 | | | | | |
| Q13200 | 26S proteasome non-ATPase | 94.27 | 31.81 | 0 | 0 | 0 |
| | regulatory subunit 2 | | | | | |
| O43242 | 26S proteasome non-ATPase | 36.37 | 13.66 | 18.64 | 0 | 0 |
| | regulatory subunit 3 | | | | | |
| P23396 | 40S ribosomal protein S3 | 109.35 | 40.56 | 0 | 0 | 0 |
| P62701 | 40S ribosomal protein S4, X | 142.61 | 24.34 | 0 | 0 | 0 |
| | isoform | | | | | |
| P46781 | 40S ribosomal protein S9 | 133.04 | 29.89 | 0 | 0 | 0 |
| P08865 | 40S ribosomal protein SA | 64.36 | 0 | 3.57 | 0 | 0 |
| P51993 | 4-galactosyl-N- | 17.18 | 43.12 | 0 | 0 | 0 |
| | acetylglucosaminide 3- | | | | | |
| | alpha-L-fucosyltransferase | | | | | |
| | FUT6 | | | | | |
| P49189 | 4-trimethylaminobutyraldehyde | 98.58 | 80.27 | 28.45 | 0 | 23.76 |
| | dehydrogenase | | | | | |
| P10809 | 60 kDa heat shock protein, | 64.13 | 43.12 | 0 | 12.62 | 0 |
| | mitochondrial | | | | | |
| P10155 | 60 kDa SS-A/Ro | 59.58 | 41.42 | 0 | 0 | 0 |
| | ribonucleoprotein | | | | | |
| P61313 | 60S ribosomal protein L15 | 211.52 | 31.6 | 0 | 0 | 0 |
| Q02878 | 60S ribosomal protein L6 | 334.98 | 11.44 | 0 | 1.59 | 0 |
| P52209 | 6-phosphogluconate | 150.5 | 497.42 | 582.75 | 0.91 | 45.29 |
| | dehydrogenase, | | | | | |
| | decarboxylating | | | | | |
| Q13510 | Acid ceramidasc | 77.76 | 173.56 | 0 | 0 | 0 |
| Q99798 | Aconitate hydratase, | 244.06 | 29.89 | 0 | 0 | 0 |
| | mitochondrial | | | | | |
| P68133 | Actin, alpha skeletal muscle | 260.81 | 7472.02 | 153.05 | 723.54 | 794.4 |
| P63261 | Actin, cytoplasmic 2 | 2186.97 | 14559.7 | 9908.76 | 2562.9 | 1259.66 |
| P61160 | Actin-related protein 2 | 154.81 | 377.87 | 365.94 | 19.53 | 0 |
| O15143 | Actin-related protein 2/3 | 272.77 | 399.22 | 507.21 | 0 | 57.91 |
| | complex subunit 1B | | | | | |
| O15144 | Actin-related protein 2/3 | 258.42 | 542.25 | 308.05 | 0 | 93.79 |
| | complex subunit 2 | | | | | |
| P59998 | Actin-related protein 2/3 | 0 | 288.21 | 280.58 | 0 | 23.71 |
| | complex subunit 4 | | | | | |
| P61158 | Actin-related protein 3 | 631.69 | 640.46 | 363.98 | 17.78 | 57.41 |
| P13798 | Acylamino-acid-releasing | 107.2 | 44.83 | 61.71 | 0 | 0 |
| | enzyme | | | | | |
| P55263 | Adenosine kinase | 0 | 39.28 | 8.7 | 0 | 0 |
| P23526 | Adenosylhomocysteinase | 528.8 | 112.51 | 133.42 | 11 | 49 |
| P54819 | Adenylate kinase 2, | 0 | 32.24 | 13.64 | 0 | 0 |
| | mitochondrial | | | | | |
| P00568 | Adenylate kinase isoenzyme | 45.94 | 12.32 | 0 | 0 | 0 |
| | 1 | | | | | |
| P30520 | Adenylosuccinate synthetase | 0 | 78.14 | 33.75 | 0 | 0 |
| | isozyme 2 | | | | | |
| Q01518 | Adenylyl cyclase-associated | 225.4 | 375.74 | 557.24 | 0 | 62.12 |
| | protein 1 | | | | | |
| Q9HDC9 | Adipocyte plasma | 36.61 | 409.89 | 338.47 | 0 | 0 |
| | membrane-associated | | | | | |
| | protein | | | | | |
| P12235 | ADP/ATP translocase 1 | 28.71 | 91.37 | 0 | 0 | 0 |
| P05141 | ADP/ATP translocase 2 | 121.31 | 91.37 | 52 | 0 | 0 |
| P12236 | ADP/ATP translocase 3 | 88.77 | 91.37 | 52 | 0 | 0 |
| P84077 | ADP-ribosylation factor 1 | 160.55 | 324.5 | 406.16 | 5.23 | 33.9 |
| P18085 | ADP-ribosylation factor 4 | 303.88 | 56.36 | 0 | 0 | 0 |
| P62330 | ADP-ribosylation factor 6 | 40.92 | 57.21 | 0 | 0 | 0 |
| P43652 | Afamin | 0 | 0 | 72.8 | 1270.99 | 77.71 |
| O00468 | Agrin | 53.6 | 27.75 | 0 | 12.94 | 0 |
| P49588 | Alanine--tRNA ligase, | 96.43 | 17.31 | 0 | 0 | 0 |
| | cytoplasmic | | | | | |
| P00326 | Alcohol dehydrogenase 1C | 1210.73 | 60.84 | 0 | 0 | 15.52 |
| P11766 | Alcohol dehydrogenase | 43.55 | 116.99 | 0 | 0 | 0 |
| | class-3 | | | | | |
| P43353 | Aldehyde dehydrogenase | 1337.55 | 467.53 | 43.07 | 0 | 43.56 |
| | family 3 member B1 | | | | | |
| P30838 | Aldehyde dehydrogenase, | 1165.27 | 2561.83 | 63.57 | 0 | 62.61 |
| | dimeric NADP-preferring | | | | | |
| P05091 | Aldehyde dehydrogenase, | 34.22 | 42.7 | 27.37 | 0 | 0 |
| | mitochondrial | | | | | |
| P14550 | Aldo-keto reductase family 1 | 51.92 | 111.01 | 36.69 | 0 | 0 |
| | member A1 | | | | | |
| P15121 | Aldo-keto reductase family 1 | 110.54 | 109.95 | 0 | 0 | 15.62 |
| | member B1 | | | | | |
| P42330 | Aldo-keto reductase family 1 | 16.08 | 146.45 | 0 | 0 | 0 |
| | member C3 | | | | | |
| P40394 | All-trans-retinol | 373.27 | 144.1 | 0 | 0 | 15.59 |
| | dehydrogenase [NAD(+)] | | | | | |
| | ADH7 | | | | | |
| P12814 | Alpha-actinin-1 | 28.71 | 550.79 | 458.16 | 14.75 | 48.51 |
| O43707 | Alpha-actinin-4 | 214.15 | 315.96 | 150.1 | 67.12 | 30.69 |
| P35611 | Alpha-adducin | 57.43 | 8.65 | 0 | 0 | 0 |
| P04745 | Alpha-amylase 1 | 7919.99 | 1989.69 | 264.89 | 0 | 38.61 |
| P06733 | Alpha-enolase | 390.02 | 1906.43 | 1520.65 | 104.96 | 269.75 |
| Q16706 | Alpha-mannosidase 2 | 34.22 | 20.99 | 0 | 0 | 0 |
| Q9NSC7 | Alpha-N- | 0 | 46.33 | 0 | 0 | 3.79 |
| | acetylgalactosaminide alpha- | | | | | |
| | 2,6-sialyltransferase 1 | | | | | |
| P54802 | Alpha-N- | 25.6 | 39.28 | 0 | 0 | 0 |
| | acetylglucosaminidase | | | | | |
| P02662 | Alpha-S1-cascin | 147.15 | 0 | 34.63 | 0 | 0 |
| Q9H4A4 | Aminopeptidase B | 204.58 | 50.38 | 41.7 | 16.49 | 32.91 |
| P15144 | Aminopeptidase N | 251.24 | 53.8 | 0 | 4.5 | 20.81 |
| P12821 | Angiotensin-converting | 5.5 | 14.11 | 0 | 3.82 | 11.41 |
| | enzyme | | | | | |
| P01019 | Angiotensinogen | 7.9 | 28.18 | 10.5 | 856.04 | 53.21 |
| P04083 | Annexin A1 | 1213.12 | 644.73 | 119.69 | 8.53 | 43.31 |
| P50995 | Annexin A11 | 78.96 | 606.3 | 113.8 | 0 | 7.55 |
| P07355 | Annexin A2 | 880.53 | 471.8 | 269.79 | 8.26 | 27.22 |
| P12429 | Annexin A3 | 44.74 | 2668.58 | 652.41 | 9.97 | 15.79 |
| P09525 | Annexin A4 | 36.37 | 24.55 | 27.96 | 0 | 0 |
| P08758 | Annexin A5 | 227.07 | 124.89 | 24.62 | 0 | 30.44 |
| P08133 | Annexin A6 | 0 | 157.34 | 477.78 | 0 | 0 |
| P03973 | Antileukoproteinase | 5766.52 | 1319.34 | 618.07 | 0 | 56.67 |
| O43747 | AP-1 complex subunit | 89.01 | 41.2 | 0 | 0 | 0 |
| | gamma-1 | | | | | |
| Q9BXS5 | AP-1 complex subunit mu-1 | 48.09 | 27.54 | 0 | 0 | 0 |
| O95831 | Apoptosis-inducing factor 1, | 71.3 | 31.17 | 0 | 0 | 0 |
| | mitochondrial | | | | | |
| O75342 | Arachidonate 12- | 0 | 5.61 | 25.7 | 0 | 0 |
| | lipoxygenase, 12R-type | | | | | |
| P20292 | Arachidonate 5- | 0 | 70.24 | 41.5 | 0 | 0 |
| | lipoxygenase-activating | | | | | |
| | protein | | | | | |
| P05089 | Arginase-1 | 178.02 | 185.95 | 142.25 | 0 | 2.06 |
| P00966 | Argininosuccinate synthase | 118.92 | 23.06 | 0 | 0 | 0 |
| Q8N512 | Arrestin domain-containing | 40.2 | 45.47 | 0 | 0 | 0 |
| | protein 1 | | | | | |
| O43776 | Asparagine--tRNA ligase, | 175.15 | 27.11 | 0 | 0 | 15.71 |
| | cytoplasmic | | | | | |
| P14868 | Aspartate--tRNA ligase, | 162.23 | 17.08 | 0 | 0 | 0 |
| | cytoplasmic | | | | | |
| P25705 | ATP synthase subunit alpha, | 483.33 | 700.23 | 19.92 | 0 | 0 |
| | mitochondrial | | | | | |
| P06576 | ATP synthase subunit beta, | 88.29 | 258.32 | 0 | 0 | 0 |
| | mitochondrial | | | | | |
| Q86UQ4 | ATP-binding cassette sub- | 63.89 | 5.19 | 0 | 0 | 0 |
| | family A member 13 | | | | | |
| P53396 | ATP-citrate synthase | 106.24 | 16.72 | 52 | 5.67 | 0 |
| P17858 | ATP-dependent 6- | 130.17 | 0 | 17.76 | 0 | 0 |
| | phosphofructokinase, liver | | | | | |
| | type | | | | | |
| Q08211 | ATP-dependent RNA | 311.06 | 13.92 | 25.7 | 1.95 | 0 |
| | helicase A | | | | | |
| O75882 | Attractin | 107.67 | 18.96 | 10.3 | 40.8 | 14.03 |
| P17213 | Bactericidal permeability- | 181.13 | 896.64 | 1049.74 | 0 | 0 |
| | increasing protein | | | | | |
| P02730 | Band 3 anion transport | 0 | 0 | 145.2 | 0 | 18.68 |
| | protein | | | | | |
| P98160 | Basement membrane- | 0 | 11.89 | 9.4 | 7.57 | 1.96 |
| | specific heparan sulfate | | | | | |
| | proteoglycan core protein | | | | | |
| P15291 | Beta-1,4- | 158.16 | 65.33 | 36.89 | 0 | 15.76 |
| | galactosyltransferase 1 | | | | | |
| P15907 | Beta-galactoside alpha-2,6- | 42.83 | 52.09 | 0 | 0 | 0 |
| | sialyltransferase 1 | | | | | |
| P08236 | Beta-glucuronidasc | 88.53 | 178.05 | 98.11 | 0 | 10.12 |
| P06865 | Beta-hexosaminidase | 0 | 92.23 | 58.18 | 0 | 0 |
| | subunit alpha | | | | | |
| O00462 | Beta-mannosidase | 94.03 | 13.58 | 0 | 0 | 1.94 |
| P07814 | Bifunctional | 108.15 | 5.57 | 0 | 0 | 3.59 |
| | glutamate/proline--tRNA | | | | | |
| | ligase | | | | | |
| P31939 | Bifunctional purine | 112.22 | 154.78 | 0 | 0 | 0 |
| | biosynthesis protein ATIC | | | | | |
| P53004 | Biliverdin reductase A | 99.06 | 55.08 | 33.06 | 0 | 12.52 |
| P43251 | Biotinidase | 46.9 | 39.07 | 0 | 45.16 | 19.3 |
| Q13867 | Bleomycin hydrolase | 0 | 61.7 | 9.91 | 12.57 | 0 |
| Q8TDL5 | BPI fold-containing family | 49529.8 | 35225.2 | 6475.03 | 54.74 | 1366.07 |
| | B member 1 | | | | | |
| Q8N4F0 | BPI fold-containing family | 3182.35 | 3116.9 | 2864.71 | 0 | 10.2 |
| | B member 2 | | | | | |
| Q96CX2 | BTB/POZ domain- | 392.41 | 90.3 | 37.28 | 30.51 | 27.47 |
| | containing protein KCTD12 | | | | | |
| P11586 | C-1-tetrahydrofolate | 59.58 | 0 | 23.94 | 0 | 0 |
| | synthase, cytoplasmic | | | | | |
| P04003 | C4b-binding protein alpha | 0 | 40.56 | 341.41 | 289.42 | 68.3 |
| | chain | | | | | |
| P27708 | CAD protein | 0 | 1.41 | 0 | 1.4 | 1.88 |
| P12830 | Cadherin-1 | 66.52 | 34.8 | 0 | 24.06 | 0 |
| P27824 | Calnexin | 94.27 | 326.63 | 256.06 | 0 | 0 |
| P07384 | Calpain-1 catalytic subunit | 614.94 | 275.4 | 223.68 | 20.4 | 42.81 |
| P17655 | Calpain-2 catalytic subunit | 313.45 | 54.23 | 0 | 20.4 | 28.71 |
| O15484 | Calpain-5 | 77.29 | 35.01 | 0 | 0 | 0 |
| P27797 | Calreticulin | 679.54 | 1212.6 | 1020.31 | 381.82 | 157.89 |
| P13861 | cAMP-dependent protein | 186.16 | 46.54 | 35.42 | 0 | 0 |
| | kinase type II-alpha | | | | | |
| | regulatory subunit | | | | | |
| P00915 | Carbonic anhydrase 1 | 0 | 0 | 1697.24 | 0 | 232.38 |
| P00918 | Carbonic anhydrase 2 | 0 | 49.74 | 300.21 | 0 | 29.45 |
| P23280 | Carbonic anhydrase 6 | 51.68 | 48.25 | 0 | 0 | 0 |
| P16152 | Carbonyl reductase | 11.94 | 20.24 | 0 | 0 | 0 |
| | [NADPH] 1 | | | | | |
| Q9UI42 | Carboxypeptidase A4 | 12.94 | 3.24 | 34.83 | 0 | 0 |
| P06731 | Carcinoembryonic antigen- | 57.43 | 8.82 | 0 | 0 | 4.92 |
| | related cell adhesion | | | | | |
| | molecule 5 | | | | | |
| Q9NQ79 | Cartilage acidic protein 1 | 17.32 | 0 | 0 | 0 | 51.72 |
| P31944 | Caspase-14 | 35.89 | 9.01 | 51.21 | 0 | 0 |
| P04040 | Catalase | 954.71 | 1477.32 | 2256.45 | 20.75 | 215.3 |
| P07858 | Cathepsin B | 94.03 | 604.17 | 80.35 | 36.26 | 0 |
| P07339 | Cathepsin D | 373.27 | 429.11 | 117.73 | 15.24 | 14.77 |
| P08311 | Cathepsin G | 892.49 | 7963.03 | 6386.73 | 0 | 18.44 |
| P25774 | Cathepsin S | 130.17 | 668.21 | 207.99 | 0 | 30.19 |
| Q9UBR2 | Cathepsin Z | 125.62 | 146.45 | 0 | 0 | 0 |
| P11717 | Cation-independent | 39.72 | 7.19 | 6.38 | 2.67 | 2.82 |
| | mannose-6-phosphate | | | | | |
| | receptor | | | | | |
| Q6YHK3 | CD109 antigen | 0 | 1.24 | 0 | 0 | 1.81 |
| Q13740 | CD166 antigen | 0 | 47.61 | 0 | 26.68 | 0 |
| O43866 | CD5 antigen-like | 0 | 28.39 | 186.4 | 0 | 0 |
| P60953 | Cell division control protein | 0 | 173.14 | 61.22 | 0 | 11.9 |
| | 42 homolog | | | | | |
| P36222 | Chitinase-3-like protein 1 | 11.58 | 420.57 | 125.58 | 0 | 71.77 |
| Q13231 | Chitotriosidase-1 | 0 | 646.86 | 240.36 | 0 | 23.54 |
| O00299 | Chloride intracellular | 406.77 | 354.39 | 57.88 | 32.78 | 34.89 |
| | channel protein 1 | | | | | |
| Q96NY7 | Chloride intracellular | 141.17 | 115.07 | 0 | 0 | 0 |
| | channel protein 6 | | | | | |
| Q53GD3 | Choline transporter-like | 413.95 | 113.15 | 0 | 0 | 13.26 |
| | protein 4 | | | | | |
| O15335 | Chondroadherin | 22.73 | 27.33 | 0 | 0 | 0 |
| Q9Y6A4 | Cilia- and flagella-associated | 33.02 | 10.59 | 0 | 0 | 0 |
| | protcin 20 | | | | | |
| Q8N1V2 | Cilia- and flagella-associated | 63.17 | 26.47 | 0 | 0 | 0 |
| | protein 52 | | | | | |
| Q00610 | Clathrin heavy chain 1 | 961.88 | 45.47 | 129.5 | 21.97 | 27.47 |
| P10909 | Clusterin | 197.64 | 3.35 | 0 | 38.88 | 0 |
| Q14019 | Coactosin-like protein | 120.83 | 196.83 | 240.36 | 0 | 156.9 |
| P53621 | Coatomer subunit alpha | 241.67 | 8.39 | 8.24 | 2.53 | 5.74 |
| P35606 | Coatomer subunit beta′ | 206.49 | 65.54 | 22.86 | 0 | 0 |
| P48444 | Coatomer subunit delta | 146.44 | 33.52 | 0 | 0 | 0 |
| Q9Y678 | Coatomer subunit gamma-1 | 94.27 | 8.56 | 0 | 0 | 0 |
| P23528 | Cofilin-1 | 199.08 | 279.67 | 347.3 | 0 | 34.65 |
| P02452 | Collagen alpha-1(I) chain | 0 | 0 | 0 | 33.3 | 3.93 |
| P12111 | Collagen alpha-3(VI) chain | 0 | 0 | 0 | 74.97 | 16.78 |
| P17927 | Complement receptor type 1 | 0 | 4.8 | 11.18 | 0 | 0 |
| Q99829 | Copine-1 | 27.04 | 93.72 | 176.59 | 0 | 0 |
| Q96FN4 | Copine-2 | 0 | 17.25 | 33.85 | 0 | 0 |
| O75131 | Copine-3 | 130.88 | 169.51 | 461.1 | 0 | 0 |
| O75367 | Core histone macro-H2A.1 | 75.13 | 127.66 | 20.21 | 0 | 0 |
| Q15517 | Corneodesmosin | 394.8 | 27.54 | 140.29 | 0 | 0 |
| P22528 | Cornifin-B | 28 | 4.27 | 24.43 | 0 | 0 |
| P31146 | Coronin-1A | 397.2 | 856.08 | 990.88 | 58.23 | 98 |
| Q9BR76 | Coronin-1B | 226.59 | 35.65 | 0 | 0 | 17.57 |
| Q9ULV4 | Coronin-1C | 194.29 | 71.73 | 74.36 | 0 | 23.83 |
| P57737 | Coronin-7 | 68.67 | 21.78 | 41.89 | 0 | 0 |
| Q86VP6 | Cullin-associated NEDD8- | 37.57 | 0 | 0 | 2.68 | 0 |
| | dissociated protein 1 | | | | | |
| P01040 | Cystatin-A | 0 | 10.61 | 96.73 | 0 | 0 |
| P01037 | Cystatin-SN | 30.63 | 245.51 | 0 | 0 | 0 |
| P54108 | Cysteine-rich secretory | 0 | 226.3 | 181.5 | 0 | 0 |
| | protein 3 | | | | | |
| P04839 | Cytochrome b-245 heavy | 0 | 100.98 | 51.31 | 0 | 0 |
| | chain | | | | | |
| Q08477 | Cytochrome P450 4F3 | 0 | 162.25 | 48.76 | 0 | 0 |
| P21399 | Cytoplasmic aconitate | 90.92 | 28.39 | 0 | 0 | 16.01 |
| | hydratasc | | | | | |
| Q14204 | Cytoplasmic dynein 1 heavy | 5862.23 | 21.99 | 14.52 | 6.12 | 80.18 |
| | chain 1 | | | | | |
| Q13409 | Cytoplasmic dynein 1 | 66.52 | 30.96 | 0 | 0 | 0 |
| | intermediate chain 2 | | | | | |
| Q8NCM8 | Cytoplasmic dynein 2 heavy | 87.1 | 15.65 | 0 | 0 | 4.28 |
| | chain 1 | | | | | |
| Q7L576 | Cytoplasmic FMR1- | 89.97 | 7.39 | 0 | 0 | 8.09 |
| | interacting protein 1 | | | | | |
| Q96F07 | Cytoplasmic FMR1- | 0 | 3.63 | 7.7 | 0 | 0 |
| | interacting protein 2 | | | | | |
| P28838 | Cytosol aminopeptidase | 63.17 | 91.59 | 0 | 115.42 | 27.72 |
| Q96KP4 | Cytosolic non-specific | 195.49 | 141.75 | 0 | 16.04 | 45.04 |
| | dipeptidase | | | | | |
| P49902 | Cytosolic purine 5′- | 0 | 33.09 | 50.72 | 0 | 0 |
| | nucleotidase | | | | | |
| Q92608 | Dedicator of cytokinesis | 0 | 3.91 | 13.24 | 0 | 1.47 |
| | protein 2 | | | | | |
| Q9UGM3 | Deleted in malignant brain | 111262.7 | 4312.42 | 2168.15 | 31.38 | 517.23 |
| | tumors 1 protein | | | | | |
| Q9Y3Z3 | Deoxynucleoside | 142.13 | 27.33 | 0 | 0 | 9.4 |
| | triphosphate | | | | | |
| | triphosphohydrolase | | | | | |
| | SAMHD1 | | | | | |
| P81605 | Dermcidin | 1569.64 | 101.19 | 1726.67 | 0 | 53.21 |
| Q08554 | Desmocollin-1 | 734.57 | 64.05 | 392.43 | 0 | 7.18 |
| Q14574 | Desmocollin-3 | 154.09 | 0 | 64.36 | 0 | 0 |
| Q02413 | Desmoglein-1 | 540.76 | 81.12 | 563.13 | 0 | 14.87 |
| P15924 | Desmoplakin | 1770.63 | 86.89 | 1932.7 | 0 | 22.15 |
| P60981 | Destrin | 92.84 | 35.23 | 0 | 0 | 0 |
| P09622 | Dihydrolipoyl | 203.14 | 93.72 | 48.37 | 0 | 0 |
| | dehydrogenase, | | | | | |
| | mitochondrial | | | | | |
| Q16555 | Dihydropyrimidinase-related | 84.94 | 57.64 | 0 | 44.46 | 177.94 |
| | protein 2 | | | | | |
| Q01459 | Di-N-acetylchitobiase | 0 | 173.14 | 36.4 | 0 | 0 |
| P53634 | Dipeptidyl peptidase 1 | 1758.67 | 2094.3 | 426.76 | 28.07 | 73.5 |
| Q9NY33 | Dipeptidyl peptidase 3 | 66.28 | 52.73 | 25.31 | 11.93 | 20.54 |
| P27487 | Dipeptidyl peptidase 4 | 127.77 | 14.3 | 0 | 5.67 | 19.5 |
| Q9P265 | Disco-interacting protein 2 | 36.37 | 2.37 | 0 | 0 | 0 |
| | homolog B | | | | | |
| Q16531 | DNA damage-binding | 191.9 | 30.32 | 0 | 2.74 | 0 |
| | protein 1 | | | | | |
| P27695 | DNA-(apurinic or | 20.39 | 234.83 | 163.84 | 0 | 0 |
| | apyrimidinic site) | | | | | |
| | endonuclease | | | | | |
| P78527 | DNA-dependent protein | 79.68 | 8.88 | 12.66 | 0 | 0 |
| | kinase catalytic subunit | | | | | |
| P59910 | DnaJ homolog subfamily B | 30.87 | 49.74 | 0 | 0 | 0 |
| | member 13 | | | | | |
| O75165 | DnaJ homolog subfamily C | 14.14 | 0 | 5.17 | 0 | 0 |
| | member 13 | | | | | |
| P39656 | Dolichyl- | 165.82 | 33.94 | 0 | 0 | 0 |
| | diphosphooligosaccharide-- | | | | | |
| | protein glycosyltransferase | | | | | |
| | 48 kDa subunit | | | | | |
| P04843 | Dolichyl- | 213.19 | 51.02 | 0 | 0 | 0 |
| | diphosphooligosaccharide-- | | | | | |
| | protein glycosyltransferase | | | | | |
| | subunit 1 | | | | | |
| P04844 | Dolichyl- | 104.08 | 29.89 | 77.99 | 0 | 0 |
| | diphosphooligosaccharide-- | | | | | |
| | protein glycosyltransferase | | | | | |
| | subunit 2 | | | | | |
| Q9UJU6 | Drebrin-like protein | 38.76 | 45.69 | 67.4 | 0 | 21.16 |
| Q14203 | Dynactin subunit 1 | 40.2 | 3.67 | 0 | 1.2 | 0 |
| Q96DT5 | Dynein heavy chain 11, | 28.95 | 3.48 | 0 | 0 | 0 |
| | axonemal | | | | | |
| Q6ZR08 | Dynein heavy chain 12, | 24.88 | 0.72 | 0 | 0 | 0 |
| | axonemal | | | | | |
| Q8TE73 | Dynein heavy chain 5, | 801.57 | 67.46 | 0 | 0 | 383.59 |
| | axonemal | | | | | |
| Q9C0G6 | Dynein heavy chain 6, | 13.83 | 1.67 | 0 | 0 | 0 |
| | axonemal | | | | | |
| Q9NYC9 | Dynein heavy chain 9, | 70.83 | 73.87 | 0 | 0 | 2.33 |
| | axonemal | | | | | |
| O75923 | Dysferlin | 0 | 7.26 | 18.05 | 0 | 0 |
| Q7Z6Z7 | E3 ubiquitin-protein ligase | 31.11 | 0 | 0 | 0.9 | 0 |
| | HUWE1 | | | | | |
| O95834 | Echinoderm microtubule- | 318.24 | 62.55 | 33.65 | 0 | 0 |
| | associated protein-like 2 | | | | | |
| O14638 | Ectonucleotide | 40.2 | 3.97 | 0 | 0 | 0 |
| | pyrophosphatase/phosphodie | | | | | |
| | sterase family member 3 | | | | | |
| Q5JST6 | EF-hand domain-containing | 94.27 | 17.1 | 0 | 0 | 0 |
| | family member C2 | | | | | |
| Q12805 | EGF-containing fibulin-like | 811.14 | 232.7 | 36.3 | 197.01 | 132.4 |
| | extracellular matrix protein 1 | | | | | |
| Q9H4M9 | EH domain-containing | 44.03 | 56.15 | 47.68 | 0 | 0 |
| | protein 1 | | | | | |
| Q9H223 | EH domain-containing | 102.41 | 58.07 | 0 | 0 | 0 |
| | protein 4 | | | | | |
| P68104 | Elongation factor 1-alpha 1 | 5407.61 | 958.55 | 671.05 | 129.19 | 559.3 |
| P26641 | Elongation factor 1-gamma | 291.92 | 268.99 | 96.05 | 16.68 | 22.99 |
| P13639 | Elongation factor 2 | 825.5 | 196.41 | 187.38 | 40.8 | 77.71 |
| P49411 | Elongation factor Tu, | 48.57 | 29.67 | 0 | 0 | 0 |
| | mitochondrial | | | | | |
| Q9NZ08 | Endoplasmic reticulum | 157.68 | 33.73 | 0 | 0 | 0 |
| | aminopeptidase 1 | | | | | |
| P11021 | Endoplasmic reticulum | 1691.67 | 358.66 | 153.05 | 114.89 | 29.94 |
| | chaperone BiP | | | | | |
| P30040 | Endoplasmic reticulum | 15.7 | 87.74 | 77.11 | 0 | 0 |
| | resident protein 29 | | | | | |
| P14625 | Endoplasmin | 1026.49 | 159.69 | 111.84 | 38.53 | 23.88 |
| P12724 | Eosinophil cationic protein | 0 | 529.45 | 158.93 | 0 | 0 |
| P11678 | Eosinophil peroxidase | 0 | 473.94 | 138.33 | 0 | 0 |
| Q8TE68 | Epidermal growth factor | 44.74 | 21.99 | 0 | 0 | 0 |
| | receptor kinase substrate 8- | | | | | |
| | like protein 1 | | | | | |
| Q9H6S3 | Epidermal growth factor | 109.35 | 10.4 | 0 | 0 | 0 |
| | receptor kinase substrate 8- | | | | | |
| | like protein 2 | | | | | |
| Q96HE7 | ERO1-like protein alpha | 0 | 45.9 | 20.31 | 0 | 0 |
| P60842 | Eukaryotic initiation factor | 145 | 90.73 | 82.21 | 0 | 19.06 |
| | 4A-I | | | | | |
| P41091 | Eukaryotic translation | 54.79 | 25.19 | 0 | 0 | 0 |
| | initiation factor 2 subunit 3 | | | | | |
| Q14152 | Eukaryotic translation | 120.59 | 1.95 | 0 | 1.81 | 2.21 |
| | initiation factor 3 subunit A | | | | | |
| P55884 | Eukaryotic translation | 96.91 | 1.46 | 0 | 2.86 | 0 |
| | initiation factor 3 subunit B | | | | | |
| Q9Y262 | Eukaryotic translation | 32.06 | 203.45 | 286.47 | 0 | 0 |
| | initiation factor 3 subunit L | | | | | |
| Q04637 | Eukaryotic translation | 23.07 | 0 | 0 | 0 | 0.88 |
| | initiation factor 4 gamma 1 | | | | | |
| P56537 | Eukaryotic translation | 12.56 | 20 | 11.77 | 0 | 0 |
| | initiation factor 6 | | | | | |
| Q9BSJ8 | Extended synaptotagmin-1 | 72.5 | 1.84 | 0 | 0 | 0 |
| Q16610 | Extracellular matrix protein | 150.03 | 28.61 | 43.36 | 51.08 | 1.28 |
| | 1 | | | | | |
| P15311 | Ezrin | 7824.28 | 1765.53 | 409.1 | 58.93 | 470.21 |
| P52907 | F-actin-capping protein | 14.43 | 123.18 | 47.78 | 0 | 0 |
| | subunit alpha-1 | | | | | |
| P47756 | F-actin-capping protein | 203.86 | 157.98 | 301.19 | 0 | 0 |
| | subunit beta | | | | | |
| P49327 | Fatty acid synthase | 159.84 | 5.47 | 41.2 | 11.19 | 17.67 |
| Q01469 | Fatty acid-binding protein 5 | 122.51 | 29.03 | 611.2 | 0 | 5.59 |
| P15090 | Fatty acid-binding protein, | 161.51 | 0 | 81.04 | 0 | 0 |
| | adipocyte | | | | | |
| Q2WGJ9 | Fer-1-like protein 6 | 14.28 | 2.39 | 0 | 0 | 0 |
| Q86UX7 | Fermitin family homolog 3 | 113.42 | 48.03 | 196.21 | 0 | 9.06 |
| P02794 | Ferritin heavy chain | 0 | 37.57 | 0 | 0 | 55.68 |
| P23142 | Fibulin-1 | 0 | 34.16 | 0 | 82.47 | 21.58 |
| P20930 | Filaggrin | 89.01 | 16.93 | 194.25 | 0 | 0 |
| Q5D862 | Filaggrin-2 | 233.53 | 54.01 | 285.49 | 0 | 3.59 |
| P21333 | Filamin-A | 509.65 | 456.86 | 501.32 | 66.43 | 58.9 |
| O75369 | Filamin-B | 433.09 | 9.31 | 5.43 | 18.31 | 8.29 |
| O75955 | Flotillin-1 | 31.34 | 35.44 | 0 | 0 | 0 |
| P09467 | Fructose-1,6-bisphosphatase | 157.68 | 388.54 | 0 | 0 | 147.5 |
| | 1 | | | | | |
| O00757 | Fructose-1,6-bisphosphatase | 0 | 260.45 | 0 | 0 | 84.14 |
| | isozyme 2 | | | | | |
| P04075 | Fructose-bisphosphate | 279.95 | 727.99 | 1010.5 | 233.62 | 123.24 |
| | aldolase A | | | | | |
| P09972 | Fructose-bisphosphate | 0 | 30.96 | 301.19 | 0 | 0 |
| | aldolase C | | | | | |
| P16930 | Fumarylacetoacetase | 0 | 95 | 89.87 | 2.68 | 19.2 |
| P17931 | Galectin-3 | 0 | 277.53 | 0 | 0 | 14.18 |
| Q08380 | Galectin-3-binding protein | 3086.64 | 1387.66 | 216.82 | 627.65 | 598.89 |
| Q92820 | Gamma-glutamyl hydrolase | 0 | 33.09 | 18.54 | 0 | 0 |
| O75223 | Gamma- | 54.55 | 39.71 | 35.81 | 0 | 0 |
| | glutamylcyclotransferase | | | | | |
| Q96QA5 | Gasdermin-A | 34.69 | 10.74 | 0 | 0 | 0 |
| P20142 | Gastricsin | 0 | 0 | 0 | 29.64 | 64.34 |
| O95479 | GDH/6PGL endoplasmic | 17.87 | 17.85 | 0 | 0 | 0 |
| | bifunctional protein | | | | | |
| P06396 | Gelsolin | 3469.48 | 3821.4 | 1952.32 | 864.76 | 1499.71 |
| P11413 | Glucose-6-phosphate 1- | 194.77 | 392.81 | 663.2 | 0 | 27.96 |
| | dehydrogenase | | | | | |
| P06744 | Glucose-6-phosphate | 142.61 | 3543.87 | 1088.98 | 87.35 | 381.11 |
| | isomerase | | | | | |
| P14314 | Glucosidase 2 subunit beta | 0 | 154.35 | 0 | 25.45 | 11.41 |
| P00367 | Glutamate dehydrogenase 1, | 339.77 | 73.23 | 0 | 0 | 0 |
| | mitochondrial | | | | | |
| P48506 | Glutamate--cysteine ligase | 99.06 | 29.89 | 0 | 0 | 10.99 |
| | catalytic subunit | | | | | |
| Q5RHP9 | Glutamate-rich protein 3 | 127.29 | 7.37 | 0 | 0 | 2.47 |
| P22352 | Glutathione peroxidase 3 | 0 | 0 | 0 | 140.52 | 3.27 |
| P00390 | Glutathione reductase, | 356.52 | 465.4 | 244.29 | 27.2 | 52.22 |
| | mitochondrial | | | | | |
| P08263 | Glutathione S-transferase A1 | 122.75 | 62.98 | 0 | 0 | 0 |
| P09210 | Glutathione S-transferase A2 | 43.31 | 62.98 | 0 | 0 | 0 |
| P09211 | Glutathione S-transferase P | 349.34 | 459 | 111.84 | 23.54 | 31.92 |
| P48637 | Glutathione synthetase | 53.6 | 138.77 | 87.41 | 21.79 | 24.38 |
| P04406 | Glyceraldehyde-3-phosphate | 1859.16 | 2177.56 | 1442.17 | 782.82 | 294.5 |
| | dehydrogenase | | | | | |
| P41250 | Glycine--tRNA ligase | 55.99 | 17.76 | 17.66 | 0 | 0 |
| P13807 | Glycogen [starch] synthase, | 0 | 30.32 | 56.41 | 0 | 0 |
| | muscle | | | | | |
| P35573 | Glycogen debranching | 104.8 | 7.6 | 9.67 | 0 | 5.42 |
| | enzyme | | | | | |
| P11216 | Glycogen phosphorylase, | 138.54 | 21.99 | 0 | 8.05 | 16.61 |
| | brain form | | | | | |
| P06737 | Glycogen phosphorylase, | 92.12 | 597.76 | 515.06 | 3.77 | 69.05 |
| | liver form | | | | | |
| P11217 | Glycogen phosphorylase, | 190.22 | 21.78 | 0 | 0 | 0 |
| | muscle form | | | | | |
| Q9HC38 | Glyoxalase domain- | 85.42 | 170.79 | 75.05 | 0 | 0 |
| | containing protein 4 | | | | | |
| P62993 | Growth factor receptor- | 0 | 57.43 | 36.2 | 0 | 0 |
| | bound protein 2 | | | | | |
| P62826 | GTP-binding nuclear protein | 425.91 | 209.86 | 62.3 | 0 | 33.16 |
| | Ran | | | | | |
| P63096 | Guanine nucleotide-binding | 66.76 | 23.48 | 0 | 0 | 0 |
| | protein G(i) subunit alpha-1 | | | | | |
| P04899 | Guanine nucleotide-binding | 62.45 | 212.85 | 154.03 | 0 | 0 |
| | protcin G(i) subunit alpha-2 | | | | | |
| P08754 | Guanine nucleotide-binding | 34.22 | 86.68 | 23.35 | 0 | 0 |
| | protein G(i) subunit alpha-3 | | | | | |
| P62873 | Guanine nucleotide-binding | 228.51 | 112.72 | 21.98 | 0 | 7.72 |
| | protein G(I)/G(S)/G(T) | | | | | |
| | subunit beta-1 | | | | | |
| P62879 | Guanine nucleotide-binding | 476.16 | 104.82 | 56.51 | 0 | 10.69 |
| | protein G(I)/G(S)/G(T) | | | | | |
| | subunit beta-2 | | | | | |
| P50148 | Guanine nucleotide-binding | 73.7 | 50.6 | 15.3 | 0 | 2.85 |
| | protein G(q) subunit alpha | | | | | |
| P29992 | Guanine nucleotide-binding | 313.45 | 89.02 | 0 | 0 | 0 |
| | protein subunit alpha-11 | | | | | |
| Q14344 | Guanine nucleotide-binding | 69.87 | 30.32 | 298.24 | 0 | 0 |
| | protein subunit alpha-13 | | | | | |
| O95837 | Guanine nucleotide-binding | 31.82 | 12.45 | 0 | 0 | 0 |
| | protein subunit alpha-14 | | | | | |
| P0DMV9 | Heat shock 70 kDa protein | 10025.6 | 610.57 | 816.25 | 60.32 | 194.52 |
| | 1B | | | | | |
| P34932 | Heat shock 70 kDa protein 4 | 270.38 | 45.47 | 27.96 | 51.26 | 11.06 |
| P11142 | Heat shock cognate 71 kDa | 4570.15 | 747.2 | 422.84 | 195.27 | 208.62 |
| | protein | | | | | |
| Q92598 | Heat shock protein 105 kDa | 368.48 | 35.44 | 0 | 27.55 | 11.51 |
| P04792 | Heat shock protein beta-1 | 180.65 | 17.01 | 143.24 | 0 | 0 |
| P07900 | Heat shock protein HSP 90- | 3110.57 | 518.77 | 361.03 | 235.37 | 242.28 |
| | alpha | | | | | |
| P08238 | Heat shock protein HSP 90- | 1569.64 | 266.86 | 159.91 | 57.19 | 78.7 |
| | beta | | | | | |
| P14317 | Hematopoietic lineage cell- | 0 | 36.72 | 28.06 | 0 | 0 |
| | specific protein | | | | | |
| P51858 | Hepatoma-derived growth | 0 | 63.62 | 49.74 | 0 | 0 |
| | factor | | | | | |
| Q5SSJ5 | Heterochromatin protein 1- | 11.99 | 41.2 | 0 | 0 | 0 |
| | binding protein 3 | | | | | |
| P09651 | Heterogeneous nuclear | 102.41 | 69.81 | 79.17 | 19 | 0 |
| | ribonucleoprotcin A1 | | | | | |
| P51991 | Heterogeneous nuclear | 123.7 | 42.7 | 75.84 | 0 | 0 |
| | ribonucleoprotein A3 | | | | | |
| Q14103 | Heterogeneous nuclear | 267.99 | 141.54 | 270.77 | 0 | 0 |
| | ribonucleoprotein DO | | | | | |
| O14979 | Heterogeneous nuclear | 47.38 | 0 | 103.01 | 0 | 0 |
| | ribonucleoprotein D-like | | | | | |
| P31943 | Heterogeneous nuclear | 38.28 | 0 | 81.92 | 0 | 0 |
| | ribonucleoprotein H | | | | | |
| P61978 | Heterogeneous nuclear | 43.79 | 79.2 | 35.91 | 0 | 0 |
| | ribonucleoprotein K | | | | | |
| P14866 | Heterogeneous nuclear | 184.72 | 49.74 | 36.5 | 0 | 0 |
| | ribonucleoprotein L | | | | | |
| O60506 | Heterogeneous nuclear | 100.5 | 29.25 | 27.27 | 0 | 0 |
| | ribonucleoprotein Q | | | | | |
| O43390 | Heterogeneous nuclear | 150.5 | 20.28 | 0 | 0 | 0 |
| | ribonucleoprotein R | | | | | |
| Q00839 | Heterogeneous nuclear | 686.72 | 82.62 | 139.31 | 0 | 0 |
| | ribonucleoprotein U | | | | | |
| Q1KMD3 | Heterogeneous nuclear | 164.86 | 41.63 | 0 | 0 | 0 |
| | ribonucleoprotein U-like | | | | | |
| | protein 2 | | | | | |
| P22626 | Heterogeneous nuclear | 406.77 | 107.6 | 95.56 | 0 | 0 |
| | ribonucleoproteins A2/B1 | | | | | |
| P07910 | Heterogeneous nuclear | 64.84 | 48.46 | 0 | 0 | 0 |
| | ribonucleoproteins C1/C2 | | | | | |
| P19367 | Hexokinase-1 | 248.85 | 12.6 | 0 | 0 | 0 |
| P52790 | Hexokinase-3 | 0 | 125.32 | 174.63 | 0 | 16.7 |
| P42357 | Histidine ammonia-lyase | 0 | 27.33 | 23.55 | 0 | 0 |
| P04196 | Histidine-rich glycoprotein | 11.29 | 112.51 | 240.36 | 458.53 | 191.55 |
| P12081 | Histidine--tRNA ligase, | 27.04 | 104.18 | 31.49 | 5.46 | 0 |
| | cytoplasmic | | | | | |
| P16403 | Histone H1.2 | 279.95 | 1063.16 | 0 | 0 | 0 |
| P0C0S8 | Histone H2A type 1 | 303.88 | 236.97 | 0 | 165.63 | 7.47 |
| P62807 | Histone H2B type 1- | 557.51 | 260.45 | 31.39 | 37.14 | 0 |
| | C/E/F/G/I | | | | | |
| P62805 | Histone H4 | 638.86 | 1146.42 | 272.74 | 35.74 | 0 |
| Q09028 | Histone-binding protein | 0 | 273.26 | 0 | 1.95 | 0 |
| | RBBP4 | | | | | |
| P04439 | HLA class I | 11.84 | 125.53 | 0 | 0 | 0 |
| | histocompatibility antigen, A | | | | | |
| | alpha chain | | | | | |
| P10321 | HLA class I | 25.6 | 143.89 | 0 | 38.53 | 0 |
| | histocompatibility antigen, C | | | | | |
| | alpha chain | | | | | |
| P01903 | HLA class II | 19.26 | 133.22 | 0 | 4.03 | 0 |
| | histocompatibility antigen, | | | | | |
| | DR alpha chain | | | | | |
| Q30154 | HLA class II | 0 | 128.31 | 0 | 3 | 0 |
| | histocompatibility antigen, | | | | | |
| | DR beta 5 chain | | | | | |
| P01911 | HLA class II | 0 | 68.96 | 0 | 3 | 0 |
| | histocompatibility antigen, | | | | | |
| | DRB1 beta chain | | | | | |
| Q86YZ3 | Hornerin | 1457.18 | 277.53 | 2550.77 | 0 | 56.18 |
| Q16543 | Hsp90 co-chaperone Cdc37 | 165.58 | 50.17 | 48.96 | 0 | 0 |
| Q14520 | Hyaluronan-binding protein | 0 | 0 | 0 | 31.56 | 25.24 |
| | 2 | | | | | |
| P00492 | Hypoxanthine-guanine | 0 | 55.93 | 19.62 | 0 | 0 |
| | phosphoribosyltransferase | | | | | |
| Q9Y4L1 | Hypoxia up-regulated | 134.47 | 12.66 | 0 | 0 | 0 |
| | protein 1 | | | | | |
| Q9Y6R7 | IgGFc-binding protein | 6532.2 | 1330.02 | 4493.28 | 0 | 190.56 |
| O00410 | Importin-5 | 4.79 | 0 | 0 | 2.88 | 0 |
| P29218 | Inositol monophosphatase 1 | 0 | 63.19 | 35.12 | 0 | 0 |
| O14732 | Inositol monophosphatase 2 | 0 | 271.13 | 21.68 | 0 | 0 |
| P14735 | Insulin-degrading enzyme | 45.22 | 0 | 26.39 | 0 | 0 |
| P18065 | Insulin-like growth factor- | 40.44 | 65.54 | 0 | 0 | 0 |
| | binding protein 2 | | | | | |
| P11215 | Integrin alpha-M | 25.12 | 292.48 | 213.87 | 4.11 | 5.15 |
| P20702 | Integrin alpha-X | 0 | 12.75 | 0 | 0 | 2.62 |
| P05107 | Integrin beta-2 | 0 | 561.47 | 598.45 | 0 | 15.71 |
| P05362 | Intercellular adhesion | 0 | 0 | 0 | 12.1 | 13.04 |
| | molecule 1 | | | | | |
| Q12905 | Interleukin enhancer-binding | 20.36 | 39.49 | 0 | 8.4 | 0 |
| | factor 2 | | | | | |
| Q96RY7 | Intraflagellar transport | 54.55 | 6.55 | 0 | 0 | 0 |
| | protein 140 homolog | | | | | |
| Q9UG01 | Intraflagellar transport | 122.27 | 9.29 | 0 | 0 | 1.07 |
| | protein 172 homolog | | | | | |
| P07476 | Involucrin | 21.22 | 3.71 | 11.48 | 0 | 0 |
| O75874 | Isocitrate dehydrogenase | 139.02 | 154.56 | 45.13 | 13.13 | 35.39 |
| | [NADP] cytoplasmic | | | | | |
| P48735 | Isocitrate dehydrogenase | 42.83 | 88.17 | 0 | 0 | 0 |
| | [NADP], mitochondrial | | | | | |
| P41252 | Isoleucine--tRNA ligase, | 114.37 | 4.29 | 0 | 0 | 2.92 |
| | cytoplasmic | | | | | |
| P14923 | Junction plakoglobin | 1311.22 | 87.74 | 649.47 | 0 | 18.31 |
| P29622 | Kallistatin | 0 | 51.66 | 38.65 | 97.46 | 65.09 |
| Q14894 | Ketimine reductase mu- | 109.83 | 40.56 | 0 | 0 | 0 |
| | crystallin | | | | | |
| Q7Z4S6 | Kinesin-like protein KIF21A | 93.56 | 0 | 0 | 0 | 1.67 |
| P01042 | Kininogen-1 | 49.53 | 163.32 | 759.34 | 1328.52 | 353.89 |
| P22079 | Lactoperoxidase | 945.13 | 380.01 | 123.61 | 0 | 0 |
| P20700 | Lamin-B1 | 69.87 | 55.93 | 25.61 | 34.87 | 0 |
| P11047 | Laminin subunit gamma-1 | 0 | 1.91 | 0 | 32.43 | 0 |
| Q32MZ4 | Leucine-rich repeat | 0 | 2.13 | 0 | 5.11 | 0 |
| | flightless-interacting protein | | | | | |
| | 1 | | | | | |
| P30740 | Leukocyte elastase inhibitor | 887.71 | 5379.85 | 4140.09 | 39.93 | 259.85 |
| Q8N6C8 | Leukocyte immunoglobulin- | 0 | 24.55 | 28.35 | 29.29 | 0 |
| | like receptor subfamily A | | | | | |
| | member 3 | | | | | |
| P09960 | Leukotriene A-4 hydrolase | 648.43 | 657.54 | 502.31 | 50.56 | 383.59 |
| Q14847 | LIM and SH3 domain | 88.77 | 60.2 | 0 | 0 | 0 |
| | protein 1 | | | | | |
| P18428 | Lipopolysaccharide-binding | 0 | 0 | 28.55 | 78.11 | 14.13 |
| | protein | | | | | |
| P23141 | Liver carboxylesterase 1 | 1387.79 | 121.9 | 440.5 | 6.59 | 9.38 |
| P00338 | L-lactate dehydrogenase A | 230.9 | 2305.65 | 694.59 | 150.98 | 119.28 |
| | chain | | | | | |
| P07195 | L-lactate dehydrogenase B | 1036.06 | 764.28 | 292.36 | 43.59 | 136.11 |
| | chain | | | | | |
| P42785 | Lysosomal Pro-X | 0 | 72.8 | 89.77 | 0 | 0 |
| | carboxypeptidase | | | | | |
| P11279 | Lysosome-associated | 84.46 | 25.83 | 20.9 | 0 | 0 |
| | membrane glycoprotein 1 | | | | | |
| P61626 | Lysozyme C | 23353.2 | 30955.49 | 5474.34 | 9.71 | 47.02 |
| Q96C86 | m7GpppX diphosphatase | 0 | 24.34 | 28.25 | 0 | 0 |
| P22897 | Macrophage mannose | 55.03 | 10.48 | 0 | 87 | 69.05 |
| | receptor 1 | | | | | |
| Q9UEW3 | Macrophage receptor | 0 | 0 | 0 | 4.43 | 41.08 |
| | MARCO | | | | | |
| P40121 | Macrophage-capping protein | 182.33 | 646.86 | 935.94 | 21.79 | 87.85 |
| Q14764 | Major vault protein | 337.38 | 61.06 | 80.74 | 0 | 13.04 |
| P40925 | Malate dehydrogenase, | 43.07 | 358.66 | 88.98 | 0 | 28.21 |
| | cytoplasmic | | | | | |
| P40926 | Malate dehydrogenase, | 12.01 | 335.17 | 44.05 | 0 | 0 |
| | mitochondrial | | | | | |
| O43451 | Maltase-glucoamylase, | 0 | 12.53 | 13.24 | 0 | 0 |
| | intestinal | | | | | |
| Q9Y5P6 | Mannose-1-phosphate | 39.96 | 35.65 | 0 | 0 | 0 |
| | guanyltransferase beta | | | | | |
| P14780 | Matrix metalloproteinase-9 | 519.23 | 4355.12 | 5955.06 | 0 | 69.05 |
| P11310 | Medium-chain specific acyl- | 64.6 | 30.32 | 0 | 0 | 0 |
| | CoA dehydrogenase, | | | | | |
| | mitochondrial | | | | | |
| P01033 | Metalloproteinase inhibitor 1 | 49.05 | 230.57 | 0 | 91.53 | 0 |
| Q687X5 | Metalloreductase STEAP4 | 263.2 | 135.99 | 0 | 0 | 9.4 |
| Q13228 | Methanethiol oxidase | 1847.2 | 333.04 | 214.85 | 0 | 137.84 |
| P27816 | Microtubule-associated | 20.79 | 0 | 0 | 1.57 | 0 |
| | protein 4 | | | | | |
| Q16539 | Mitogen-activated protein | 0 | 23.91 | 17.17 | 0 | 0 |
| | kinase 14 | | | | | |
| P26038 | Moesin | 406.77 | 2134.86 | 2158.34 | 86.48 | 356.37 |
| P22234 | Multifunctional protein | 0 | 27.54 | 8.56 | 0 | 0 |
| | ADE2 | | | | | |
| P24158 | Myeloblastin | 22.23 | 2040.93 | 1775.73 | 46.72 | 267.28 |
| P41218 | Myeloid cell nuclear | 226.11 | 871.02 | 2344.75 | 0 | 17.52 |
| | differentiation antigen | | | | | |
| P05164 | Myeloperoxidase | 591.01 | 4291.07 | 3217.89 | 39.23 | 98.25 |
| Q9NZM1 | Myoferlin | 227.79 | 16.78 | 0 | 0 | 5.67 |
| Q7Z406 | Myosin-14 | 320.63 | 12.66 | 8.81 | 0 | 6.41 |
| P35579 | Myosin-9 | 6962.89 | 337.31 | 602.37 | 1600.5 | 3167.71 |
| O14745 | Na(+)/H(+) exchange | 152.66 | 234.83 | 80.94 | 0 | 0 |
| | regulatory cofactor NHE- | | | | | |
| | RF1 | | | | | |
| Q9UJ70 | N-acetyl-D-glucosamine | 34.22 | 58.07 | 43.26 | 9.22 | 0 |
| | kinase | | | | | |
| P34059 | N-acetylgalactosamine-6- | 0 | 35.44 | 23.64 | 0 | 0 |
| | sulfatase | | | | | |
| Q86SF2 | N- | 98.1 | 324.5 | 0 | 0 | 0 |
| | acetylgalactosaminyltransferase | | | | | |
| | 7 | | | | | |
| Q96PD5 | N-acetylmuramoyl-L- | 0 | 0 | 0 | 114.37 | 47.02 |
| | alanine amidase | | | | | |
| P15559 | NAD(P)H dehydrogenase | 25.12 | 499.56 | 0 | 0 | 30.93 |
| | [quinone] 1 | | | | | |
| P23368 | NAD-dependent malic | 0 | 49.74 | 29.04 | 0 | 0 |
| | enzyme, mitochondrial | | | | | |
| O96009 | Napsin-A | 41.39 | 32.02 | 0 | 66.95 | 135.62 |
| O43847 | Nardilysin | 0 | 0 | 0 | 3.68 | 2.01 |
| Q9Y2A7 | Nck-associated protein 1 | 112.7 | 2.48 | 0 | 0 | 0 |
| Q09666 | Neuroblast differentiation- | 8015.7 | 18.68 | 42.09 | 5.13 | 14.06 |
| | associated protein AHNAK | | | | | |
| O60462 | Neuropilin-2 | 58.62 | 4.29 | 0 | 0 | 40.34 |
| Q14697 | Neutral alpha-glucosidase | 483.33 | 74.93 | 68.38 | 9.17 | 16.9 |
| | AB | | | | | |
| P22894 | Neutrophil collagenase | 47.85 | 1667.33 | 1393.11 | 0 | 20.54 |
| P14598 | Neutrophil cytosol factor 1 | 0 | 138.34 | 248.21 | 0 | 0 |
| P19878 | Neutrophil cytosol factor 2 | 0 | 42.06 | 88.98 | 0 | 0 |
| Q15080 | Neutrophil cytosol factor 4 | 0 | 58.07 | 132.44 | 0 | 0 |
| P59665 | Neutrophil defensin 1 | 770.46 | 77.28 | 1216.52 | 9.17 | 69.79 |
| P08246 | Neutrophil elastase | 156.25 | 5849.52 | 6524.08 | 0 | 403.39 |
| P80188 | Neutrophil gelatinase- | 248.85 | 2732.62 | 478.76 | 0 | 0 |
| | associated lipocalin | | | | | |
| P43490 | Nicotinamide | 35.65 | 215.62 | 123.61 | 0 | 10.69 |
| | phosphoribosyltransferase | | | | | |
| Q6XQN6 | Nicotinate | 0 | 40.56 | 0 | 0 | 0.79 |
| | phosphoribosyltransferase | | | | | |
| Q15233 | Non-POU domain- | 58.86 | 15.24 | 0 | 0 | 0 |
| | containing octamer-binding | | | | | |
| | protein | | | | | |
| Q9Y266 | Nuclear migration protein | 76.09 | 0 | 0 | 10.22 | 0 |
| | nudC | | | | | |
| P19338 | Nucleolin | 799.18 | 118.06 | 210.93 | 72.18 | 0 |
| Q9NTK5 | Obg-like ATPase 1 | 32.06 | 25.62 | 0 | 0 | 0 |
| Q6UX06 | Olfactomedin-4 | 0 | 63.19 | 324.73 | 0 | 0 |
| Q9NQR4 | Omega-amidase NIT2 | 19.86 | 60.84 | 0 | 0 | 0 |
| Q99497 | Parkinson disease protein 7 | 37.09 | 115.28 | 0 | 0 | 0 |
| P26022 | Pentraxin-related protein | 0 | 134.07 | 104.97 | 9.97 | 0 |
| | PTX3 | | | | | |
| P19021 | Peptidyl-glycinc alpha- | 294.31 | 185.31 | 90.55 | 0 | 24.67 |
| | amidating monooxygenase | | | | | |
| P23284 | Peptidyl-prolyl cis-trans | 116.53 | 426.97 | 185.42 | 0 | 0 |
| | isomerase B | | | | | |
| Q15063 | Periostin | 0 | 0 | 32.47 | 32.43 | 0 |
| O60437 | Periplakin | 116.05 | 3.63 | 0 | 0 | 0 |
| Q06830 | Peroxiredoxin-1 | 1146.13 | 802.71 | 310.02 | 9.34 | 54.94 |
| P32119 | Peroxiredoxin-2 | 241.67 | 97.99 | 924.16 | 0 | 25 |
| Q13162 | Peroxiredoxin-4 | 213.67 | 83.47 | 0 | 6.75 | 0 |
| P30044 | Peroxiredoxin-5, | 20.79 | 17.08 | 0 | 0 | 0 |
| | mitochondrial | | | | | |
| P30041 | Peroxiredoxin-6 | 332.59 | 260.45 | 208.97 | 0 | 29.45 |
| P51659 | Peroxisomal multifunctional | 111.5 | 30.32 | 0 | 0 | 0 |
| | enzymc type 2 | | | | | |
| P30086 | Phosphatidylethanolamine- | 19.02 | 54.65 | 0 | 0 | 0 |
| | binding protein 1 | | | | | |
| P36871 | Phosphoglucomutase-1 | 18.64 | 93.08 | 59.55 | 4.74 | 12.08 |
| Q96G03 | Phosphoglucomutase-2 | 45.7 | 142.18 | 34.83 | 0 | 0 |
| P00558 | Phosphoglycerate kinase 1 | 235.69 | 414.16 | 208.97 | 103.74 | 102.46 |
| Q6P4A8 | Phospholipase B-like 1 | 0 | 283.94 | 120.67 | 0 | 0 |
| P55058 | Phospholipid transfer protein | 291.92 | 191.92 | 33.06 | 280.7 | 31.92 |
| O15067 | Phosphoribosylformylglycin | 0 | 4.36 | 0 | 4.66 | 5.67 |
| | amidine synthase | | | | | |
| Q9GZP4 | PITH domain-containing | 0 | 28.82 | 20.41 | 0 | 0 |
| | protein 1 | | | | | |
| Q13835 | Plakophilin-1 | 311.06 | 18.02 | 154.03 | 0 | 0 |
| P03952 | Plasma kallikrein | 0 | 0 | 24.92 | 183.06 | 44.55 |
| P13796 | Plastin-2 | 218.46 | 1626.76 | 1010.5 | 292.9 | 239.56 |
| Q13093 | Platelet-activating factor | 0 | 3.97 | 0 | 7.99 | 0 |
| | acetylhydrolase | | | | | |
| P43034 | Platelet-activating factor | 194.53 | 67.46 | 40.42 | 0 | 17.22 |
| | acetylhydrolase IB subunit | | | | | |
| | beta | | | | | |
| Q15149 | Plectin | 579.04 | 31.38 | 32.96 | 6.89 | 12.7 |
| O00592 | Podocalyxin | 194.77 | 12.34 | 0 | 0 | 8.91 |
| P11940 | Polyadenylate-binding | 198.6 | 17.06 | 0 | 15.26 | 15.07 |
| | protein 1 | | | | | |
| PO1833 | Polymeric immunoglobulin | 23137.86 | 39921.91 | 6082.6 | 174.35 | 6805.62 |
| | receptor | | | | | |
| Q14435 | Polypeptide N- | 87.1 | 20.94 | 0 | 0 | 0 |
| | acetylgalactosaminyltransferase | | | | | |
| | 3 | | | | | |
| Q7Z7M9 | Polypeptide N- | 0 | 0 | 0 | 2.68 | 2.77 |
| | acetylgalactosaminyltransferase | | | | | |
| | 5 | | | | | |
| Q8NCL4 | Polypeptide N- | 287.13 | 148.8 | 0 | 0 | 0 |
| | acetylgalactosaminyltransferase | | | | | |
| | 6 | | | | | |
| P0CG47 | Polyubiquitin-B | 2332.93 | 962.82 | 901.6 | 51.61 | 174.72 |
| P09917 | Polyunsaturated fatty acid 5- | 38.52 | 87.96 | 72.89 | 0 | 0 |
| | lipoxygenase | | | | | |
| P16050 | Polyunsaturated fatty acid | 1093.49 | 116.78 | 0 | 0 | 9.08 |
| | lipoxygenase ALOX15 | | | | | |
| P20742 | Pregnancy zone protein | 27.52 | 2.18 | 53.57 | 138.95 | 0 |
| P02545 | Prelamin-A/C | 80.64 | 47.18 | 56.51 | 96.07 | 0 |
| Q6P2Q9 | Pre-mRNA-processing- | 33.26 | 3.59 | 0 | 0 | 0 |
| | splicing factor 8 | | | | | |
| Q92841 | Probable ATP-dependent | 21.87 | 25.62 | 39.93 | 0 | 0 |
| | RNA helicase DDX17 | | | | | |
| A0A0C4D | Probable non-functional | 0 | 158.62 | 0 | 0 | 225.95 |
| H36 | immunoglobulin heavy | | | | | |
| | variable 3-38 | | | | | |
| Q93008 | Probable ubiquitin carboxyl- | 41.87 | 4.29 | 0 | 1.07 | 2.16 |
| | terminal hydrolase FAF-X | | | | | |
| P09668 | Pro-cathepsin H | 28.95 | 56.57 | 0 | 7.13 | 18.59 |
| P07737 | Profilin-1 | 263.2 | 1238.22 | 752.48 | 20.05 | 52.22 |
| Q8WUM4 | Programmed cell death 6- | 223 | 309.55 | 11.87 | 2.06 | 6.11 |
| | interacting protein | | | | | |
| P12273 | Prolactin-inducible protein | 715.43 | 1289.46 | 223.68 | 0 | 0 |
| Q9UQ80 | Proliferation-associated | 94.27 | 94.36 | 83.49 | 0 | 0 |
| | protcin 2G4 | | | | | |
| Q07954 | Prolow-density lipoprotein | 20.7 | 4.12 | 0 | 8.33 | 5.72 |
| | receptor-related protein 1 | | | | | |
| P48147 | Prolyl endopeptidase | 47.38 | 36.29 | 0 | 0 | 0 |
| O43490 | Prominin-1 | 306.27 | 31.81 | 0 | 0 | 2.9 |
| P25789 | Proteasome subunit alpha | 61.49 | 138.98 | 52 | 49.17 | 15.07 |
| | type-4 | | | | | |
| P60900 | Proteasome subunit alpha | 65.8 | 254.05 | 119.69 | 71.13 | 28.71 |
| | type-6 | | | | | |
| O14818 | Proteasome subunit alpha | 56.71 | 160.76 | 33.16 | 12.12 | 0 |
| | type-7 | | | | | |
| P20618 | Proteasome subunit beta | 0 | 93.29 | 29.24 | 18.83 | 0 |
| | type-1 | | | | | |
| P49721 | Protcasome subunit beta | 0 | 68.53 | 16.19 | 0 | 0 |
| | type-2 | | | | | |
| P49720 | Proteasome subunit beta | 0 | 81.12 | 0 | 13.77 | 0 |
| | type-3 | | | | | |
| P28070 | Proteasome subunit beta | 0 | 89.02 | 0 | 28.24 | 0 |
| | type-4 | | | | | |
| P28074 | Proteasome subunit beta | 28.47 | 17.21 | 20.41 | 0 | 0 |
| | type-5 | | | | | |
| P28072 | Proteasome subunit beta | 33.98 | 0 | 26.98 | 0 | 0 |
| | type-6 | | | | | |
| P02760 | Protein AMBP | 0 | 11.08 | 13.05 | 207.47 | 42.57 |
| O60610 | Protein diaphanous homolog | 0 | 7.19 | 0 | 0 | 3.42 |
| | 1 | | | | | |
| P07237 | Protein disulfide-isomerase | 105.04 | 448.32 | 249.19 | 19.88 | 22.12 |
| P30101 | Protein disulfide-isomerase | 198.84 | 380.01 | 134.41 | 12.01 | 28.71 |
| | A3 | | | | | |
| P13667 | Protein disulfide-isomerase | 70.59 | 153.5 | 0 | 0 | 0 |
| | A4 | | | | | |
| Q15084 | Protein disulfide-isomerase | 37.33 | 100.13 | 19.92 | 12 | 0 |
| | A6 | | | | | |
| Q13045 | Protein flightless-1 homolog | 68.91 | 5.83 | 25.9 | 0 | 2.99 |
| Q05655 | Protein kinase C delta typc | 23.88 | 0 | 40.22 | 0 | 0 |
| Q6P5S2 | Protein LEG1 homolog | 643.65 | 764.28 | 65.44 | 0 | 0 |
| Q9UKK3 | Protein mono-ADP- | 46.9 | 3.37 | 0 | 0 | 2.3 |
| | ribosyltransferase PARP4 | | | | | |
| Q9BZQ8 | Protein Niban 1 | 67.95 | 3.24 | 0 | 2.51 | 0 |
| Q96TA1 | Protein Niban 2 | 28.23 | 19.17 | 0 | 4.24 | 0 |
| Q9UFN0 | Protein NipSnap homolog | 0 | 65.11 | 28.25 | 0 | 0 |
| | 3A | | | | | |
| Q8WVV4 | Protein POF1B | 77.05 | 6.43 | 63.47 | 0 | 0 |
| P31949 | Protein S100-A11 | 284.74 | 21.78 | 65.34 | 0 | 0 |
| Q9HCY8 | Protein S100-A14 | 43.55 | 6.34 | 25.21 | 0 | 0 |
| P31151 | Protein S100-A7 | 67.48 | 0 | 59.94 | 0 | 0 |
| P05109 | Protein S100-A8 | 1182.02 | 1515.75 | 366.92 | 92.58 | 24.75 |
| P06702 | Protein S100-A9 | 325.41 | 1099.45 | 636.71 | 0 | 0 |
| O94979 | Protein transport protein | 72.98 | 2.6 | 0 | 0 | 0 |
| | Sec31A | | | | | |
| Q70J99 | Protein unc-13 homolog D | 0 | 2.63 | 23.94 | 0 | 0 |
| Q9Y2J8 | Protein-arginine deiminase | 0 | 123.4 | 181.5 | 0 | 0 |
| | type-2 | | | | | |
| Q9UM07 | Protein-arginine deiminase | 0 | 281.8 | 531.74 | 0 | 0 |
| | type-4 | | | | | |
| P21980 | Protein-glutamine gamma- | 1694.06 | 81.12 | 7.31 | 0 | 89.09 |
| | glutamyltransferase 2 | | | | | |
| Q08188 | Protein-glutamine gamma- | 433.09 | 74.93 | 93.79 | 0 | 0 |
| | glutamyltransferase E | | | | | |
| P22735 | Protein-glutamine gamma- | 62.93 | 10.33 | 49.64 | 0 | 0 |
| | glutamyltransferase K | | | | | |
| P00491 | Purine nucleoside | 23.86 | 275.4 | 119.69 | 0 | 0 |
| | phosphorylase | | | | | |
| P55786 | Puromycin-sensitive | 275.17 | 56.79 | 50.82 | 1.81 | 17.82 |
| | aminopeptidase | | | | | |
| Q5VTE0 | Putative elongation factor 1- | 5407.61 | 958.55 | 671.05 | 129.19 | 559.3 |
| | alpha-like 3 | | | | | |
| Q9UKY3 | Putative inactive | 0 | 136.42 | 90.36 | 0 | 0 |
| | carboxylesterase 4 | | | | | |
| A6NI72 | Putative neutrophil cytosol | 0 | 138.34 | 248.21 | 0 | 0 |
| | factor 1B | | | | | |
| O00764 | Pyridoxal kinase | 0 | 67.03 | 30.81 | 0 | 7.2 |
| P14618 | Pyruvate kinase PKM | 2237.22 | 1524.29 | 3414.11 | 207.47 | 517.23 |
| P31150 | Rab GDP dissociation | 66.04 | 149.01 | 0 | 0 | 6.09 |
| | inhibitor alpha | | | | | |
| P50395 | Rab GDP dissociation | 382.84 | 537.99 | 295.3 | 10.65 | 75.73 |
| | inhibitor beta | | | | | |
| Q9H1X1 | Radial spoke head protein 9 | 18.59 | 58.71 | 0 | 0 | 0 |
| | homolog | | | | | |
| P35241 | Radixin | 100.73 | 57 | 18.64 | 16.14 | 5.67 |
| P46940 | Ras GTPase-activating-like | 892.49 | 144.1 | 137.35 | 12 | 37.12 |
| | protein IQGAP1 | | | | | |
| Q13576 | Ras GTPase-activating-like | 83.27 | 7.62 | 0 | 0 | 1.34 |
| | protein IQGAP2 | | | | | |
| P15153 | Ras-related C3 botulinum | 120.36 | 228.43 | 189.35 | 0 | 0 |
| | toxin substrate 2 | | | | | |
| P61026 | Ras-related protein Rab-10 | 115.09 | 57.43 | 96.44 | 5 | 28.95 |
| Q15907 | Ras-related protein Rab-11B | 42.59 | 82.19 | 46.8 | 0 | 0 |
| P61106 | Ras-related protein Rab-14 | 56.23 | 27.33 | 0 | 0 | 0 |
| P62820 | Ras-related protein Rab-1A | 66.52 | 0 | 97.22 | 0 | 0 |
| Q9H0U4 | Ras-related protein Rab-1B | 12.97 | 64.9 | 0 | 0 | 0 |
| Q9UL25 | Ras-related protein Rab-21 | 0 | 14.67 | 54.94 | 0 | 0 |
| P51159 | Ras-related protein Rab-27A | 0 | 11.02 | 31.49 | 0 | 0 |
| P61019 | Ras-related protein Rab-2A | 112.22 | 29.89 | 48.46 | 0 | 0 |
| P20336 | Ras-related protein Rab-3A | 373.27 | 158.41 | 0 | 0 | 0 |
| O95716 | Ras-related protein Rab-3D | 52.4 | 35.87 | 43.36 | 0 | 0 |
| P51148 | Ras-related protein Rab-5C | 31.58 | 52.09 | 72.7 | 0 | 0 |
| P51149 | Ras-related protein Rab-7a | 59.34 | 150.93 | 79.37 | 0 | 0 |
| P61006 | Ras-related protein Rab-8A | 39.48 | 28.82 | 24.92 | 0 | 0 |
| Q92930 | Ras-related protein Rab-8B | 0 | 9.09 | 55.72 | 0 | 0 |
| P11233 | Ras-related protein Ral-A | 44.98 | 36.72 | 0 | 0 | 0 |
| P11234 | Ras-related protein Ral-B | 14.6 | 60.2 | 40.13 | 0 | 0 |
| P61224 | Ras-related protein Rap-1b | 0 | 62.55 | 133.42 | 0 | 0 |
| P63244 | Receptor of activated protein | 339.77 | 53.59 | 0 | 0 | 0 |
| | C kinase 1 | | | | | |
| P08575 | Receptor-type tyrosine- | 0 | 46.33 | 34.04 | 3.56 | 3.07 |
| | protein phosphatase C | | | | | |
| Q13332 | Receptor-type tyrosine- | 52.64 | 0 | 0 | 1.28 | 0 |
| | protein phosphatase S | | | | | |
| P00352 | Retinal dehydrogenase 1 | 2632.02 | 584.95 | 61.32 | 16.11 | 203.92 |
| P49788 | Retinoic acid receptor | 161.75 | 130.65 | 0 | 0 | 0 |
| | responder protein 1 | | | | | |
| P02753 | Retinol-binding protein 4 | 211.28 | 94.36 | 27.96 | 599.75 | 112.6 |
| P52565 | Rho GDP-dissociation | 143.33 | 365.06 | 267.83 | 0 | 0 |
| | inhibitor 1 | | | | | |
| P52566 | Rho GDP-dissociation | 337.38 | 1509.35 | 3610.32 | 23.89 | 32.17 |
| | inhibitor 2 | | | | | |
| Q8N392 | Rho GTPase-activating | 33.02 | 24.12 | 0 | 0 | 13.17 |
| | protein 18 | | | | | |
| Q13464 | Rho-associated protein | 55.03 | 4.36 | 8.21 | 0 | 0 |
| | kinase 1 | | | | | |
| P34096 | Ribonuclease 4 | 351.73 | 64.26 | 0 | 0 | 0 |
| P13489 | Ribonuclease inhibitor | 109.11 | 64.69 | 50.33 | 35.92 | 15.99 |
| Q9P2E9 | Ribosome-binding protein 1 | 158.88 | 5.49 | 6.16 | 1.7 | 0 |
| Q9Y265 | RuvB-like 1 | 150.98 | 45.9 | 0 | 0 | 5 |
| Q9Y230 | RuvB-like 2 | 206.02 | 43.12 | 0 | 0 | 0 |
| Q86VB7 | Scavenger receptor cysteine- | 128.49 | 26.9 | 0 | 716.56 | 75.73 |
| | rich type 1 protein M130 | | | | | |
| Q9NVA2 | Septin-11 | 50.25 | 26.05 | 0 | 0 | 0 |
| Q15019 | Septin-2 | 88.29 | 17.61 | 0 | 0 | 0 |
| Q92743 | Serine protease HTRA1 | 602.97 | 84.54 | 45.23 | 0 | 0 |
| O94804 | Serine/threonine-protein | 0 | 3.07 | 16.29 | 0 | 0 |
| | kinase 10 | | | | | |
| P63151 | Serine/threonine-protein | 53.36 | 16.05 | 0 | 0 | 0 |
| | phosphatase 2A 55 kDa | | | | | |
| | regulatory subunit B alpha | | | | | |
| | isoform | | | | | |
| P67775 | Serine/threonine-protein | 11.46 | 64.47 | 73.58 | 0 | 0 |
| | phosphatase 2A catalytic | | | | | |
| | subunit alpha isoform | | | | | |
| Q08209 | Serine/threonine-protein | 0 | 25.19 | 41.11 | 0 | 0 |
| | phosphatase 2B catalytic | | | | | |
| | subunit alpha isoform | | | | | |
| Q9BRF8 | Serine/threonine-protein | 0 | 150.72 | 40.91 | 0 | 0 |
| | phosphatase CPPED1 | | | | | |
| P62140 | Serine/threonine-protein | 76.57 | 97.99 | 100.07 | 0 | 0 |
| | phosphatase PP1-beta | | | | | |
| | catalytic subunit | | | | | |
| P02743 | Serum amyloid P- | 0 | 153.92 | 394.39 | 127.27 | 24.65 |
| | component | | | | | |
| P27169 | Serum | 0 | 0 | 27.47 | 458.53 | 13.91 |
| | paraoxonase/arylesterase 1 | | | | | |
| P04278 | Sex hormone-binding | 0 | 0 | 14.72 | 100.08 | 3.56 |
| | globulin | | | | | |
| Q9NR45 | Sialic acid synthase | 251.24 | 73.44 | 0 | 0 | 0 |
| Q5T750 | Skin-specific protein 32 | 38.76 | 111.87 | 516.04 | 0 | 0 |
| P62314 | Small nuclear | 0 | 54.23 | 35.32 | 0 | 0 |
| | ribonucleoprotein Sm D1 | | | | | |
| P05023 | Sodium/potassium- | 3589.12 | 91.59 | 0 | 0 | 0 |
| | transporting ATPase subunit | | | | | |
| | alpha-1 | | | | | |
| O95436 | Sodium-dependent | 344.56 | 84.75 | 0 | 8.72 | 24.6 |
| | phosphate transport protein | | | | | |
| | 2B | | | | | |
| Q8WVQ1 | Soluble calcium-activated | 15.98 | 29.67 | 0 | 0 | 0 |
| | nucleotidase 1 | | | | | |
| Q16348 | Solute carrier family 15 | 37.09 | 206.01 | 0 | 0 | 0 |
| | member 2 | | | | | |
| Q00796 | Sorbitol dehydrogenase | 258.42 | 25.62 | 0 | 0 | 0 |
| Q13813 | Spectrin alpha chain, non- | 80.16 | 8.16 | 49.84 | 4.24 | 1.82 |
| | erythrocytic 1 | | | | | |
| P11277 | Spectrin beta chain, | 0 | 0 | 4.6 | 0 | 3.59 |
| | crythrocytic | | | | | |
| Q01082 | Spectrin beta chain, non- | 134.47 | 19.02 | 0 | 7.99 | 1.49 |
| | erythrocytic 1 | | | | | |
| Q13838 | Spliceosome RNA helicase | 234.97 | 75.79 | 1128.22 | 0 | 0 |
| | DDX39B | | | | | |
| Q15393 | Splicing factor 3B subunit 3 | 91.64 | 4.72 | 0 | 0 | 0 |
| P23246 | Splicing factor, proline- and | 171.32 | 19.88 | 0 | 0 | 0 |
| | glutamine-rich | | | | | |
| Q7KZF4 | Staphylococcal nuclease | 145.72 | 2.41 | 0 | 0 | 0 |
| | domain-containing protein 1 | | | | | |
| Q9H2G2 | STE20-like serine/threonine- | 112.46 | 6.45 | 0 | 0 | 2.6 |
| | protein kinase | | | | | |
| P27105 | Stomatin | 617.33 | 431.24 | 408.12 | 3.45 | 45.78 |
| P38646 | Stress-70 protcin, | 1548.11 | 34.16 | 0 | 0 | 0 |
| | mitochondrial | | | | | |
| P31948 | Stress-induced- | 22.4 | 68.1 | 88.3 | 0 | 16.95 |
| | phosphoprotein 1 | | | | | |
| Q9UQE7 | Structural maintenance of | 0 | 3.71 | 8.28 | 0 | 0 |
| | chromosomes protein 3 | | | | | |
| P31040 | Succinate dehydrogenase | 187.83 | 26.05 | 0 | 0 | 0 |
| | [ubiquinone] flavoprotein | | | | | |
| | subunit, mitochondrial | | | | | |
| O00391 | Sulfhydryl oxidase 1 | 428.3 | 412.03 | 98.11 | 1628.4 | 209.37 |
| Q9Y6N5 | Sulfide: quinone | 62.93 | 48.46 | 0 | 0 | 0 |
| | oxidoreductase, | | | | | |
| | mitochondrial | | | | | |
| Q6UWP8 | Suprabasin | 116.05 | 2.9 | 42.87 | 0 | 0 |
| Q99536 | Synaptic vesicle membrane | 55.27 | 82.83 | 80.94 | 0 | 0 |
| | protein VAT-1 homolog | | | | | |
| Q15833 | Syntaxin-binding protein 2 | 32.54 | 49.32 | 25.51 | 0 | 0 |
| Q9Y490 | Talin-1 | 171.8 | 37.79 | 70.93 | 3.73 | 15.24 |
| P17987 | T-complex protein 1 subunit | 46.42 | 32.45 | 20.7 | 0 | 0 |
| | alpha | | | | | |
| P50991 | T-complex protcin 1 subunit | 69.15 | 31.6 | 30.61 | 0 | 0 |
| | delta | | | | | |
| P48643 | T-complex protein 1 subunit | 85.66 | 32.45 | 19.13 | 0 | 29.94 |
| | epsilon | | | | | |
| Q99832 | T-complex protein 1 subunit | 54.79 | 26.9 | 34.63 | 0 | 0 |
| | eta | | | | | |
| P49368 | T-complex protein 1 subunit | 65.8 | 30.32 | 0 | 0 | 0 |
| | gamma | | | | | |
| P50990 | T-complex protein 1 subunit | 120.12 | 51.02 | 61.32 | 0 | 0 |
| | theta | | | | | |
| P40227 | T-complex protein 1 subunit | 122.27 | 19.94 | 179.53 | 0 | 5.96 |
| | zeta | | | | | |
| P24821 | Tenascin | 0 | 151.79 | 19.13 | 53.87 | 13.56 |
| Q7Z4L5 | Tetratricopeptide repeat | 12.08 | 3.35 | 0 | 0 | 0 |
| | protcin 21B | | | | | |
| P10599 | Thioredoxin | 64.36 | 105.04 | 142.25 | 0 | 0 |
| Q16881 | Thioredoxin reductase 1, | 111.74 | 148.59 | 77.11 | 29.29 | 22.3 |
| | cytoplasmic | | | | | |
| P30048 | Thioredoxin-dependent | 35.65 | 121.9 | 51.02 | 0 | 0 |
| | peroxide reductase, | | | | | |
| | mitochondrial | | | | | |
| O43396 | Thioredoxin-like protein 1 | 144.76 | 20.03 | 0 | 0 | 0 |
| P26639 | Threonine--tRNA ligase 1, | 111.5 | 0 | 44.83 | 0 | 0 |
| | cytoplasmic | | | | | |
| P07996 | Thrombospondin-1 | 90.45 | 13.71 | 0 | 13.2 | 0 |
| P19971 | Thymidine phosphorylase | 14.79 | 0 | 0 | 63.64 | 0 |
| P05543 | Thyroxine-binding globulin | 0 | 0 | 54.15 | 948.45 | 86.86 |
| Q9UDY2 | Tight junction protein ZO-2 | 20.53 | 0 | 0 | 0 | 0.81 |
| P04066 | Tissue alpha-L-fucosidase | 137.82 | 144.74 | 0 | 0 | 0 |
| P37837 | Transaldolase | 57.67 | 213.49 | 77.01 | 0 | 0 |
| P02786 | Transferrin receptor protein | 158.64 | 31.6 | 0 | 11.19 | 51.23 |
| | 1 | | | | | |
| Q15582 | Transforming growth factor- | 0 | 59.78 | 0 | 132.85 | 191.05 |
| | beta-induced protein ig-h3 | | | | | |
| P61586 | Transforming protein RhoA | 56.71 | 64.26 | 76.33 | 0 | 0 |
| P55072 | Transitional endoplasmic | 2512.38 | 179.76 | 159.91 | 0 | 37.37 |
| | reticulum ATPase | | | | | |
| P29401 | Transketolase | 1885.48 | 4419.16 | 1324.44 | 160.22 | 549.4 |
| Q7Z404 | Transmembrane channel-like | 378.05 | 169.08 | 0 | 0 | 0 |
| | protein 4 | | | | | |
| Q6UXY8 | Transmembrane channel-like | 605.36 | 87.74 | 0 | 0 | 27.22 |
| | protein 5 | | | | | |
| P49755 | Transmembrane emp24 | 113.89 | 0 | 39.93 | 0 | 0 |
| | domain-containing protein | | | | | |
| | 10 | | | | | |
| P02766 | Transthyretin | 0 | 0 | 0 | 713.08 | 69.79 |
| P40939 | Trifunctional enzymc | 181.37 | 21.56 | 0 | 0 | 0 |
| | subunit alpha, mitochondrial | | | | | |
| P55084 | Trifunctional enzyme | 130.4 | 31.38 | 0 | 0 | 0 |
| | subunit beta, mitochondrial | | | | | |
| P60174 | Triosephosphate isomerase | 61.02 | 47.39 | 36.01 | 0 | 0 |
| P29144 | Tripeptidyl-peptidase 2 | 155.29 | 5.17 | 0 | 3.36 | 4.53 |
| P23381 | Tryptophan--tRNA ligase, | 117.72 | 264.72 | 80.05 | 421.92 | 26.73 |
| | cytoplasmic | | | | | |
| Q71U36 | Tubulin alpha-1A chain | 3971.96 | 1300.13 | 341.41 | 20.05 | 284.6 |
| P68366 | Tubulin alpha-4A chain | 45.7 | 222.03 | 131.46 | 0 | 0 |
| P07437 | Tubulin beta chain | 38.76 | 77.92 | 56.22 | 60.15 | 44.79 |
| P04350 | Tubulin beta-4A chain | 0 | 339.44 | 19.03 | 0 | 42.07 |
| P68371 | Tubulin beta-4B chain | 533.58 | 832.6 | 120.67 | 30.51 | 76.22 |
| Q6IBS0 | Twinfilin-2 | 0 | 61.06 | 15.21 | 0 | 0 |
| P07948 | Tyrosine-protein kinase Lyn | 156.72 | 59.56 | 0 | 0 | 8.86 |
| P29350 | Tyrosine-protein | 68.91 | 81.55 | 126.56 | 0 | 12.4 |
| | phosphatase non-receptor | | | | | |
| | type 6 | | | | | |
| O75643 | U5 small nuclear | 25.6 | 3.18 | 0 | 0 | 0 |
| | ribonucleoprotein 200 kDa | | | | | |
| | helicase | | | | | |
| P54578 | Ubiquitin carboxyl-terminal | 32.78 | 14.07 | 0 | 0 | 0 |
| | hydrolase 14 | | | | | |
| P45974 | Ubiquitin carboxyl-terminal | 65.08 | 0 | 14.81 | 0 | 0 |
| | hydrolase 5 | | | | | |
| Q93009 | Ubiquitin carboxyl-terminal | 46.66 | 3.35 | 0 | 0 | 0 |
| | hydrolase 7 | | | | | |
| P62979 | Ubiquitin-40S ribosomal | 2332.93 | 962.82 | 901.6 | 51.61 | 174.72 |
| | protein S27a | | | | | |
| P22314 | Ubiquitin-like modifier- | 535.98 | 144.1 | 112.82 | 7.43 | 23.14 |
| | activating enzyme 1 | | | | | |
| O60701 | UDP-glucose 6- | 378.05 | 35.01 | 0 | 0 | 0 |
| | dehydrogenase | | | | | |
| Q9NYU2 | UDP-glucose: glycoprotein | 107.43 | 13.39 | 0 | 3.36 | 2.5 |
| | glucosyltransferase 1 | | | | | |
| P30085 | UMP-CMP kinase | 97.62 | 34.8 | 0 | 0 | 0 |
| Q8N0U7 | Uncharacterized protcin | 26.56 | 18.64 | 0 | 0 | 0 |
| | C1orf87 | | | | | |
| O43795 | Unconventional myosin-Ib | 258.42 | 83.69 | 0 | 0 | 0 |
| O00159 | Unconventional myosin-Ic | 81.83 | 13.24 | 0 | 0 | 0 |
| O94832 | Unconventional myosin-Id | 485.73 | 55.08 | 0 | 0 | 0 |
| O00160 | Unconventional myosin-If | 4618 | 26.69 | 67.1 | 0 | 0 |
| Q9NQX4 | Unconventional myosin-Vc | 406.77 | 13.32 | 0 | 0 | 2.34 |
| Q9UM54 | Unconventional myosin-VI | 476.16 | 12.06 | 0 | 0 | 3.56 |
| Q6UX73 | UPF0764 protein C16orf89 | 37.09 | 14.99 | 0 | 0 | 0 |
| Q16851 | UTP--glucose-1-phosphate | 275.17 | 116.56 | 110.86 | 111.93 | 25 |
| | uridylyltransferase | | | | | |
| Q709C8 | Vacuolar protein sorting- | 43.07 | 0 | 0 | 0 | 0.28 |
| | associated protein 13C | | | | | |
| O75436 | Vacuolar protein sorting- | 50.49 | 55.29 | 42.97 | 0 | 0 |
| | associated protein 26A | | | | | |
| Q96QK1 | Vacuolar protein sorting- | 71.06 | 47.18 | 51.41 | 0 | 0 |
| | associated protein 35 | | | | | |
| P26640 | Valine--tRNA ligase | 73.46 | 4.21 | 0 | 0 | 2.15 |
| P50552 | Vasodilator-stimulated | 0 | 185.31 | 146.18 | 0 | 0 |
| | phosphoprotein | | | | | |
| Q00341 | Vigilin | 146.68 | 2.9 | 0 | 0 | 1.98 |
| P08670 | Vimentin | 26.32 | 6.43 | 11.48 | 0 | 0.92 |
| P18206 | Vinculin | 121.79 | 10.16 | 17.95 | 21.27 | 31.92 |
| P02774 | Vitamin D-binding protein | 26.56 | 1530.7 | 1903.27 | 5387.31 | 767.18 |
| P07225 | Vitamin K-dependent | 0 | 67.03 | 42.48 | 15.76 | 0 |
| | protein S | | | | | |
| Q7Z5L0 | Vitelline membrane outer | 12.11 | 30.32 | 0 | 0 | 0 |
| | layer protein 1 homolog | | | | | |
| PO4004 | Vitronectin | 0 | 0 | 103.99 | 362.64 | 54.44 |
| P21796 | Voltage-dependent anion- | 112.22 | 80.48 | 508.19 | 0 | 0 |
| | selective channel protein 1 | | | | | |
| Q13303 | Voltage-gated potassium | 0 | 55.08 | 52.59 | 0 | 0 |
| | channel subunit beta-2 | | | | | |
| P38606 | V-type proton ATPase | 69.63 | 53.37 | 51.6 | 11.59 | 13.64 |
| | catalytic subunit A | | | | | |
| Q14508 | WAP four-disulfide core | 260.81 | 65.33 | 25.7 | 0 | 0 |
| | domain protein 2 | | | | | |
| O75083 | WD repeat-containing | 1112.63 | 388.54 | 593.54 | 0 | 133.89 |
| | protein 1 | | | | | |
| Q8NEZ3 | WD repeat-containing | 60.78 | 4.01 | 0 | 0 | 0 |
| | protein 19 | | | | | |
| Q9P2L0 | WD repeat-containing | 49.05 | 5.38 | 0 | 0 | 0 |
| | protein 35 | | | | | |
| Q9NQW7 | Xaa-Pro aminopeptidase 1 | 85.42 | 7.98 | 0 | 0 | 0 |
| P12955 | Xaa-Pro dipeptidase | 92.12 | 44.83 | 0 | 20.92 | 27.47 |
| P13010 | X-ray repair cross- | 157.44 | 49.32 | 39.54 | 5.84 | 0 |
| | complementing protein 5 | | | | | |
| P12956 | X-ray repair cross- | 166.77 | 62.98 | 75.54 | 7.72 | 0 |
| | complementing protein 6 | | | | | |
| P25311 | Zinc-alpha-2-glycoprotein | 311.06 | 367.2 | 232.51 | 76.36 | 6.9 |
| Q96DA0 | Zymogen granule protein 16 | 873.35 | 4162.98 | 801.53 | 0 | 0 |
| | homolog B | | | | | |
| Q15942 | Zyxin | 0 | 18.96 | 57.69 | 0 | 0 |

To further quantify the inflammatory increase of fibrinogen and prothrombin during SARS-CoV-2 infection, their concentrations were measured together with total IgG from 15 healthy, 4 acute, and 7 recovered COVID samples using ELISA. Overall, the fibrinogen, prothrombin, and total IgG concentrations measured from COVID-recovered samples were not statistically different from those of healthy donors (FIG. 1F). In contrast, both fibrinogen and prothrombin were 50-100 fold elevated in the acute COVID samples, consistent with increased risk of SARS-COV-2 infection-induced lung fibrosis. Thus, compared to healthy lungs, the acute COVID lung contained signatures of acute response protein, inflammatory infiltration of plasma proteins, coagulation factors, as well as innate immune components. The concentration of many of these inflammatory proteins in the COVID-recovered samples appeared to return to levels similar to the healthy samples.

### Example 3

### Infected Lung Epithelial Cells Induced Fibrin Clot Formation

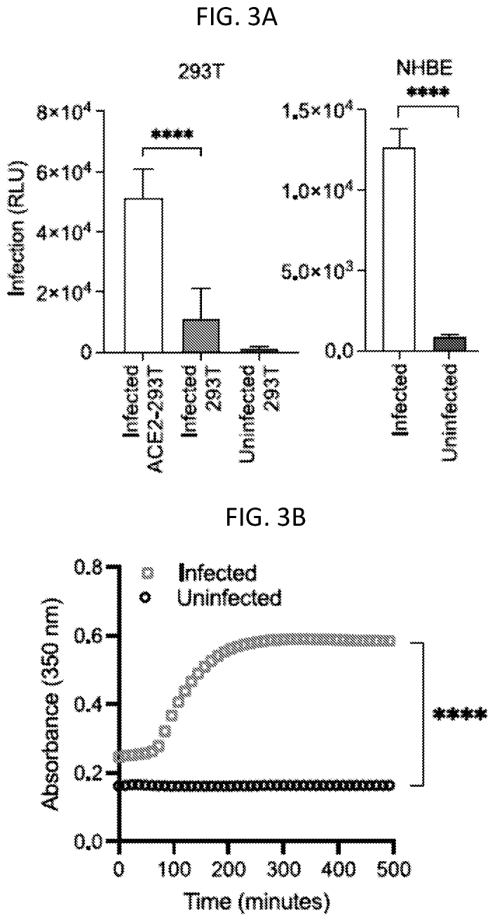

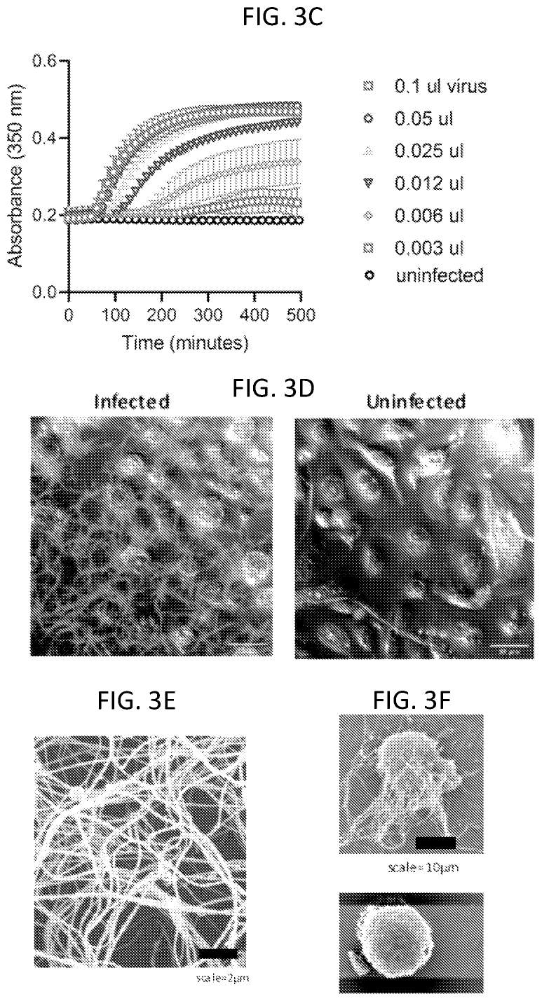

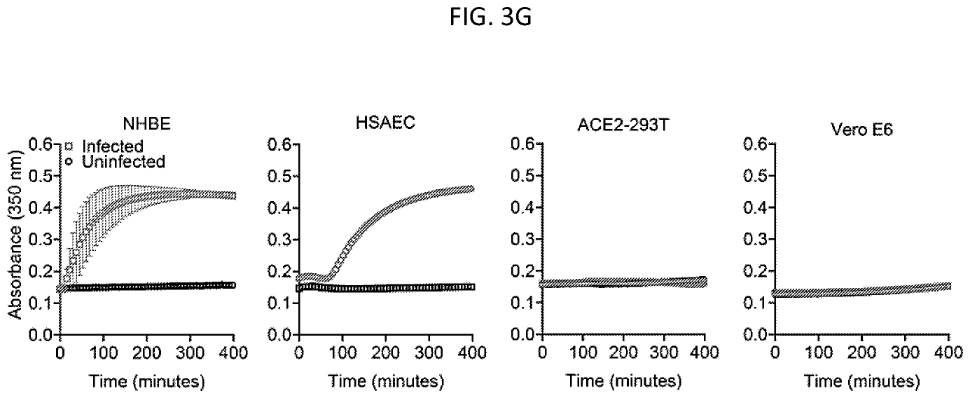

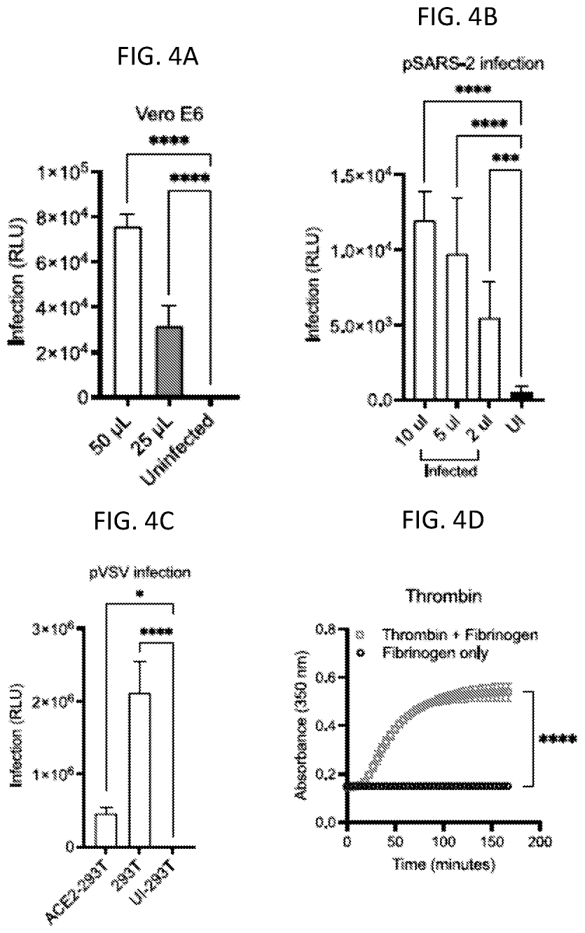

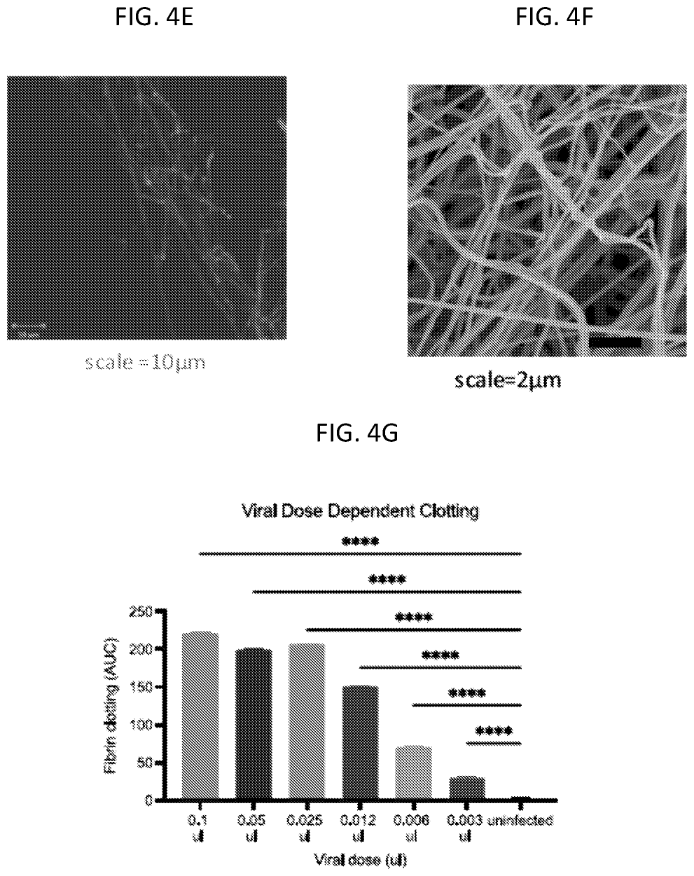

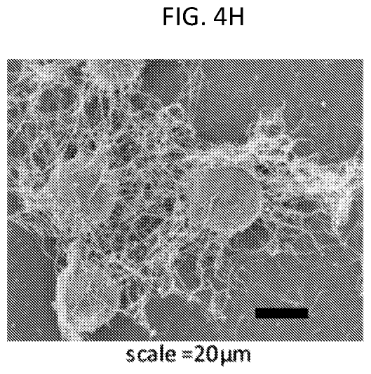

Much of the understanding of COVID-associated lung fibrosis is based on research of acute respiratory distress syndrome (ARDS). To investigate the link between viral infection and lung fibrosis, NHBE cells that are permissive to SARS-COV-2 infection were used as a model system. These were infected with both replication incompetent SARS-COV-2 pseudoviruses (pSARS-2), as well as replication competent field variants. All pSARS-2 viruses were generated by co-transfecting a variant-specific spike-expressing plasmid with a luciferase-expressing HIV core plasmid. Both ACE2-expressing 293T and NHBE cells were readily infected by a SARS-COV-2 pseudovirus (pSARS-2), expressing the prototypic Wuhan strain envelope spike protein (FIG. 3A, FIGS. 4A-4C).

SARS-COV-2 infections induce cellular and inflammatory responses in COVID lungs, but their relationship to lung fibrosis remains speculative. To characterize fibrinogen-mediated fibrosis, a turbidity-based fibrin clotting assay was adopted to measure fibrin aggregation resulting from cleavage of fibrinogen peptides (FIG. 4D). The 50-200 nm thick fibrin fiber structures formed upon thrombin cleavage of fibrinogen were visible by confocal and electron microscopy (FIGS. 4E and 4F). Fibrin clots formed by the extrinsic coagulation pathway are generally initiated with platelet aggregation and tissue factor activation. To investigate if SARS-COV-2 coronavirus infection could induce fibrin clot formation, NHBE cells were infected with the Wuhan pSARS-2 and fibrinogen was added to the infected cells. Surprisingly, the infected but not uninfected NHBE cells induced fibrin clot formation proportional to the pSARS-2 dose (FIGS. 3B and 3C, FIG. 4G). The fibrin fibers formed in the presence of the infected NHBE cells were visible in both confocal and scanning electron microscopy images (FIGS. 3D and 3E). Interestingly, many fibrin fibers were found to originate from NHBE cells in the infected sample (FIG. 3F, FIG. 4G), indicating a cell-mediated fibrin clotting mechanism induced by the viral infection. The pSARS-2 infection-induced fibrin clot formation, however, appeared unique to lung epithelial cells as both infected NHBE and human small airway epithelial cells (HSAEC) induced fibrin clot formation (FIG. 3G). Neither infected Vero-E6 nor infected ACE2-293T cells induced fibrin clot formation (FIG. 3G, FIG. 4A).

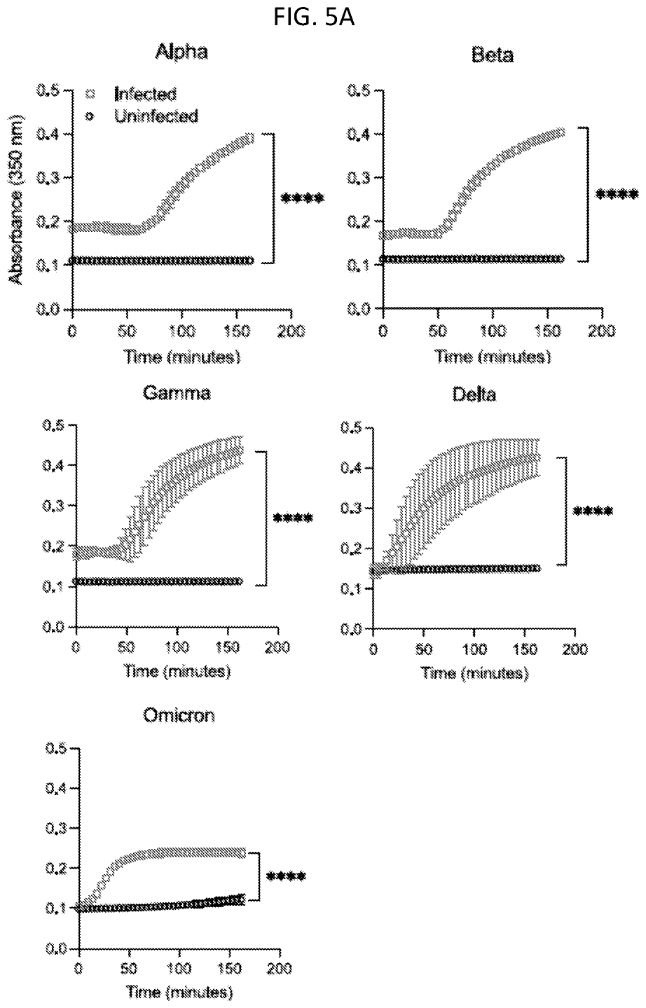

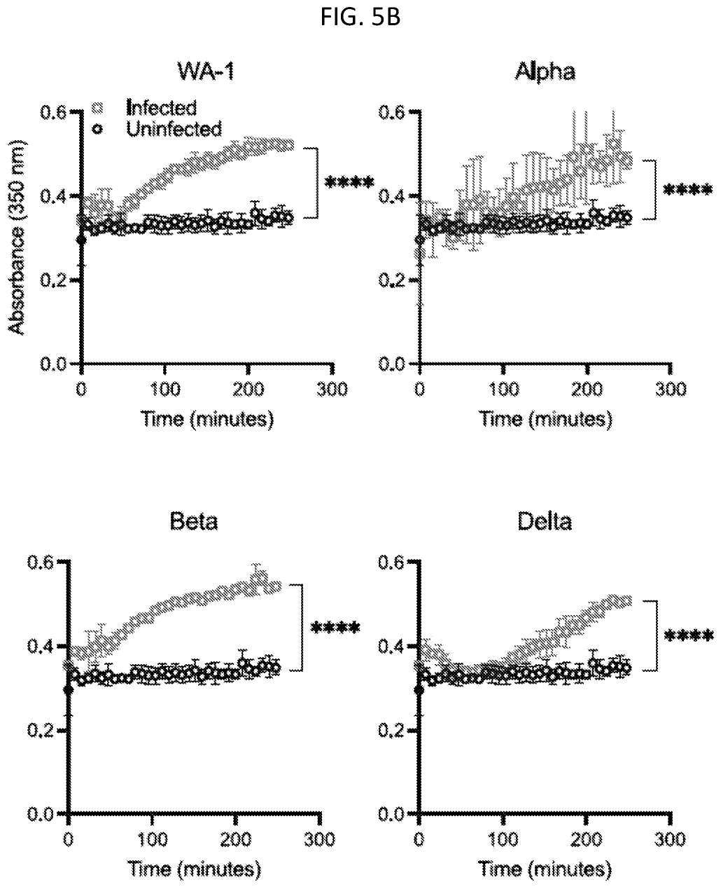

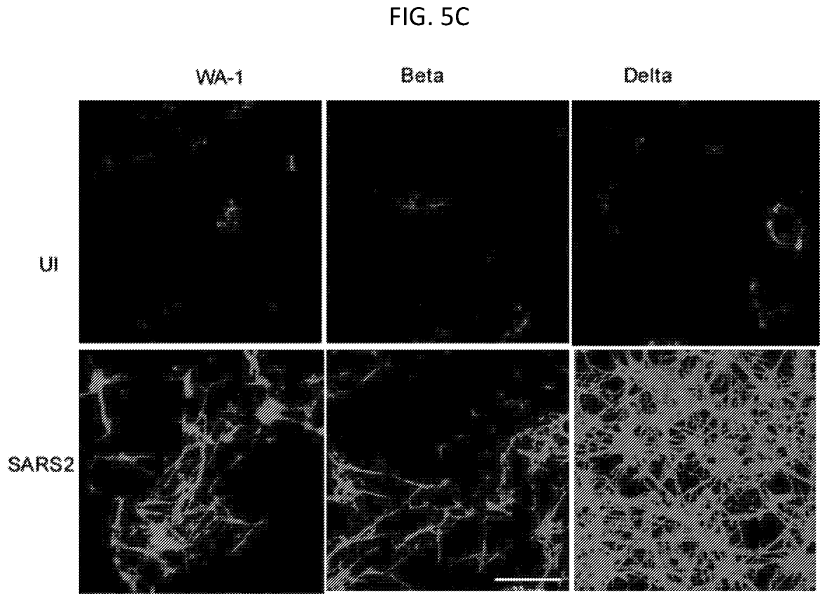

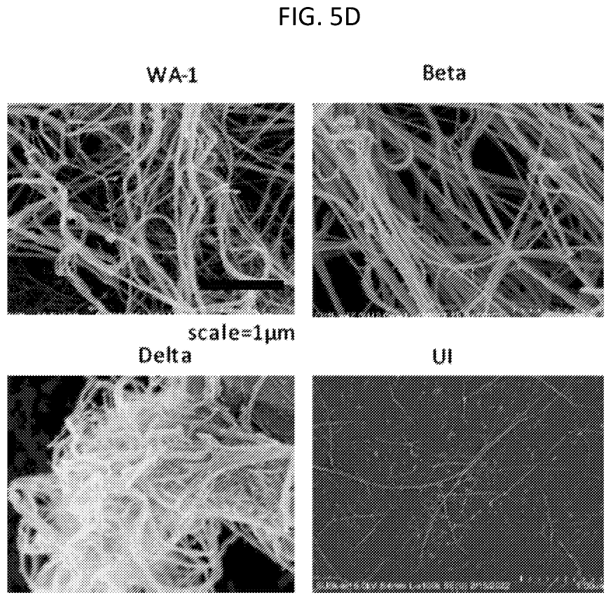

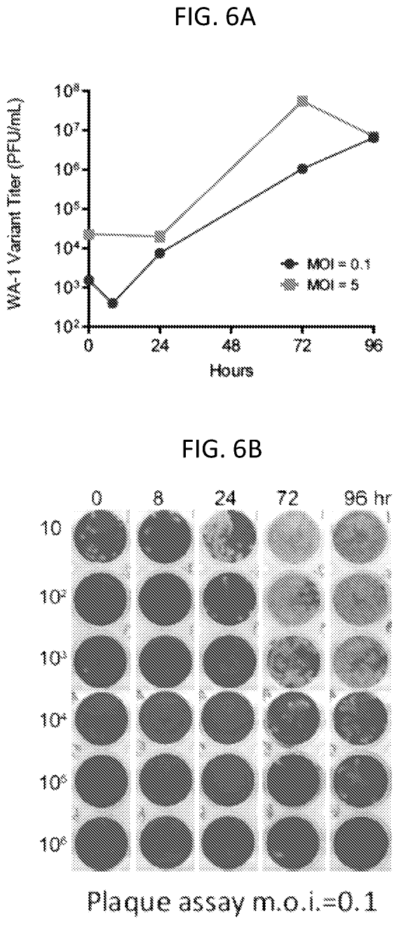

Further infections using alpha (UK), beta (South Africa), gamma (Brazil), delta, and omicron variant spike-typed pSARS-2 viruses showed that this infection-induced fibrin clot formation was broadly observed in all pseudotyped variants (FIG. 5A). To address if fibrin clot formations can be induced by replication competent circulating strains of SARS-COV-2 infections, the infection of air-liquid interface cultured NHBE cells with the Washington (WA-1) strain of SARS-COV-2 was examined and robust expansion of the virus in infected NHBE cells was observed (FIGS. 6A and 6B). Importantly, NHBE cells infected with circulating Washington (WA-1), alpha, beta, and delta strains of SARS-COV-2 supported fibrin clot formations in the infected but not uninfected cells (FIG. 5B). The infection-induced fibrin clots were visible by both confocal and scanning electron microscopy (FIGS. 5C and 5D). The structures of these fibrin clots showed extensive fibrotic network with dense fibers of 50-200 nm in thickness, similar to thrombin-induced fibers (FIGS. 5C and 5D, FIG. 4). Thus, SARS-COV-2 infections of primary human bronchial epithelial cells induced a cell-based fibrin aggregation, consistent with COVID-induced lung fibrosis widely observed throughout the world and across multiple variants.

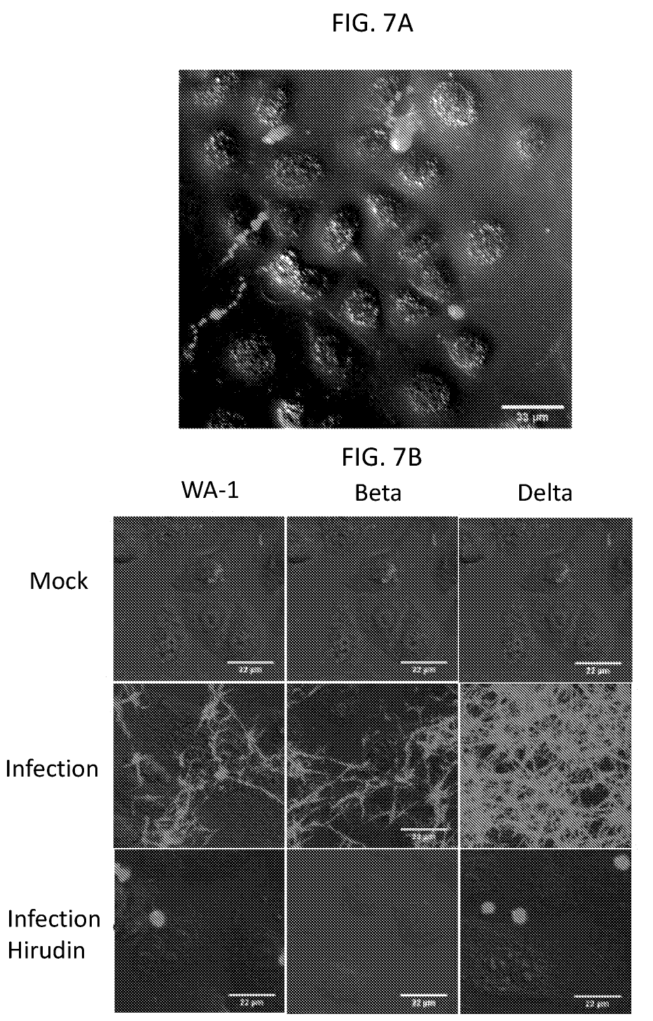

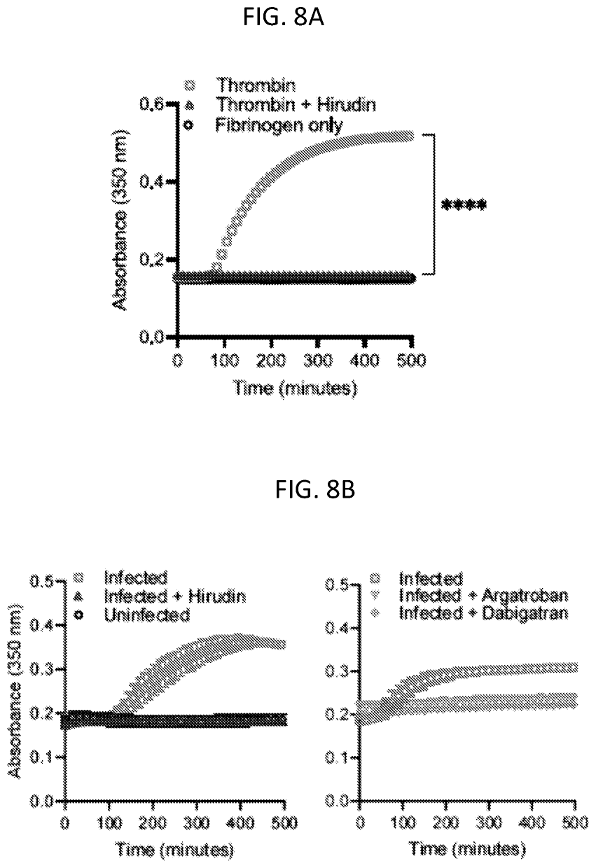

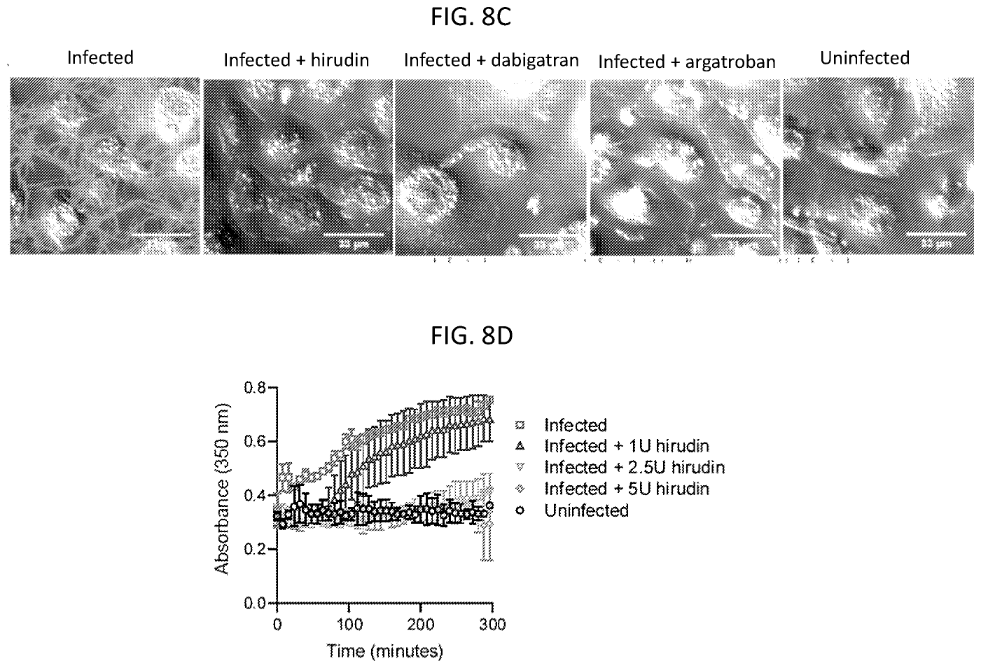

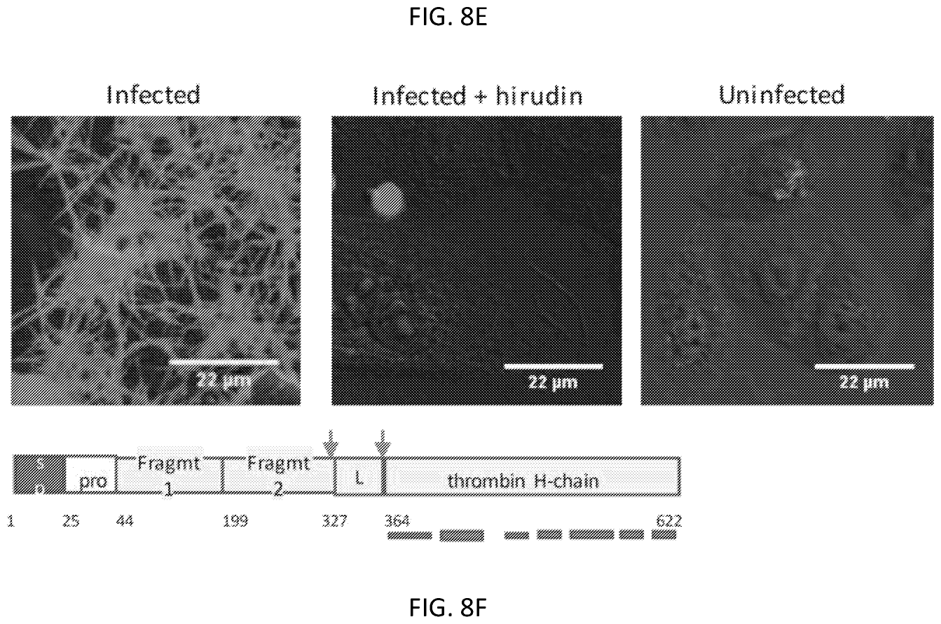

It was not clear, however, if the fibrin clot formation induced by SARS-COV-2 infection of NHBE cells required thrombin. To address this, the fibrin clotting assays were performed on pSARS-2 infected NHBE cells in the presence of a serine protease inhibitor, camostat, or a thrombin-specific inhibitor, hirudin. Both camostat and hirudin completely suppressed the infection-induced fibrin clot formation, similar to that of thrombin-induced clotting (FIG. 7A, FIGS. 8A-C). Similarly, two small molecule thrombin inhibitors, dabigatran and argatroban, also inhibited the infection-induced fibrin clotting (FIGS. 8B and 8C). Consistently, hirudin also inhibited fibrin clotting induced by replication competent WA-1, beta, and delta strains of SARS-CoV-2 infection of NHBE cells (FIGS. 8D and 8E, FIG. 7B), suggesting the infection-induced fibrin clotting was thrombin dependent. This thrombin-dependent fibrin clot formation by infected NHBE cells was a surprise as no thrombin was added to the infection and clotting assays. To address if thrombin was indeed involved in the infection-induced fibrin clotting, mass spectrometry-based protein identification analysis was performed on both infected and uninfected NHBE culture supernatants. Interestingly, multiple peptides derived from bovine thrombin were present in the infected but not uninfected NHBE culture supernatants (FIG. 8F), suggesting a bovine additive in the cell culture media as a likely source for thrombin.

### Example 4

### SARS-COV-2 Induced Thrombosis Requires Infection-Induced Release of Serine Proteases

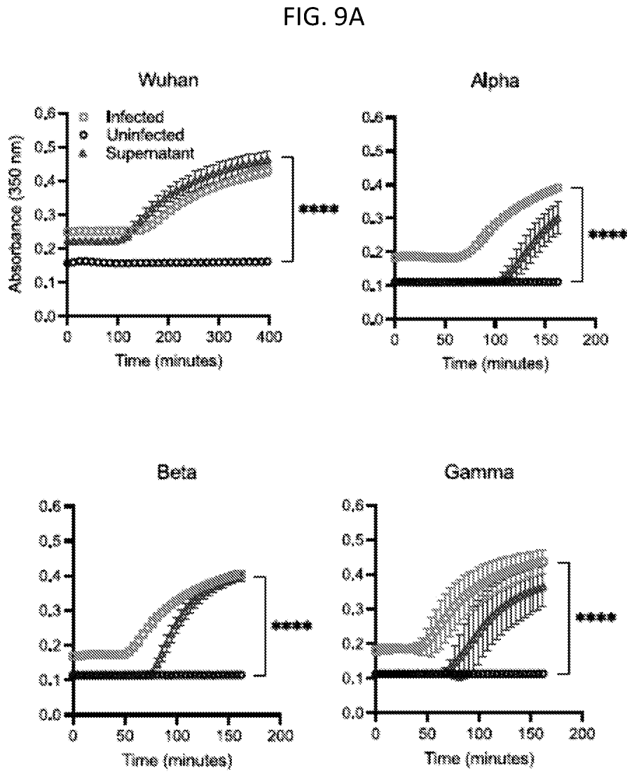

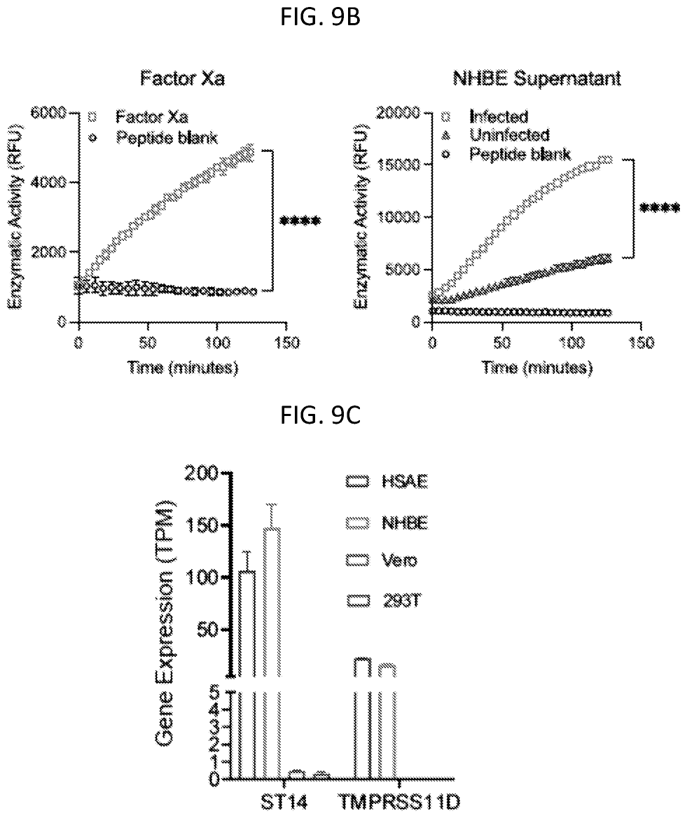

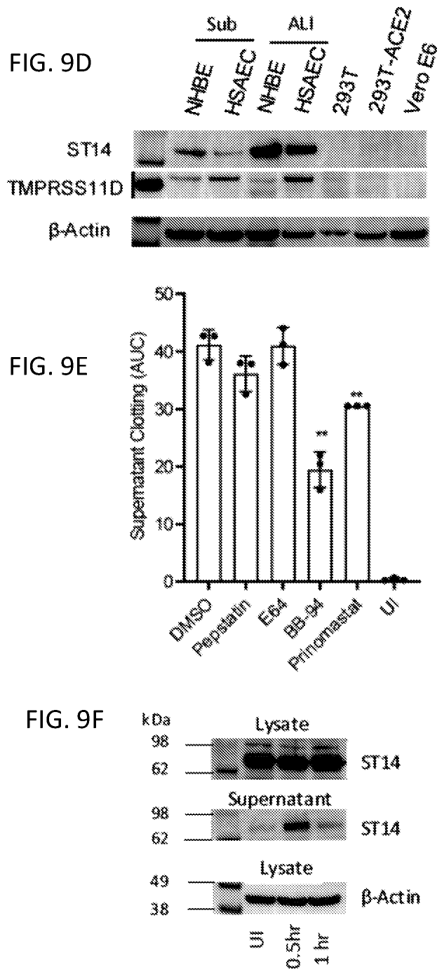

Thrombin circulates as an inactive prothrombin in plasma, therefore it must be activated by coagulation factor Xa as part of the classical coagulation pathway. It was not clear how prothrombin was activated during SARS-COV-2 infection of NHBE cells. Interestingly, the culture supernatants from infected but not uninfected NHBE cells induced fibrin clot formation (FIG. 9A), suggesting that infected NHBE cells released proteases capable of functionally activating prothrombin. To address if the infected supernatant activated prothrombin, a fluorogenic peptide corresponding to the factor Xa cleavage region of prothrombin (amino acids 324-333, referred to as Thrb-324) was synthesized. Factor Xa readily cleaved the prothrombin peptide, Thrb-324. Additionally, the infected NHBE cell supernatant showed significantly higher cleavage of Thrb-324 than the uninfected supernatant (FIG. 9B), suggesting the presence of proteases in the infected supernatant to activate prothrombin. Although tissue factor was upregulated in SARS-COV-2 infected NHBE cells, it was not clear if this leads to the cleavage of prothrombin in our in vitro infection model. As many fibrin fibers originated from infected cell surface, the potential involvement of type II transmembrane serine proteases, such as matriptase and human airway trypsin-like protease (HAT) in prothrombin activation was investigated. Both matriptase and HAT are known to be upregulated in idiopathic pulmonary fibrosis. mRNA sequencing and Western blot analyses revealed that both ST14 and TMPRSS11D, genes encoding matriptase and HAT, respectively, were expressed in NHBE and HSAEC but not in Vero and 293T cells (FIGS. 9C and 9D). The mouse homolog of ST14, epithin, was previously shown to be shed by ADAM17 in response to inflammatory stimulation and human matriptase activation required proteolytic cleavage.

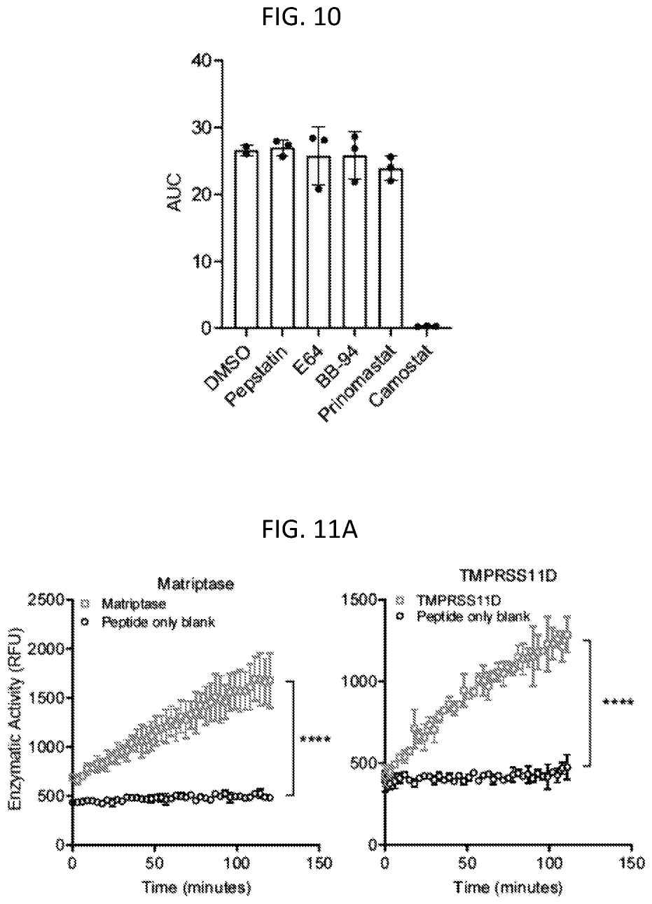

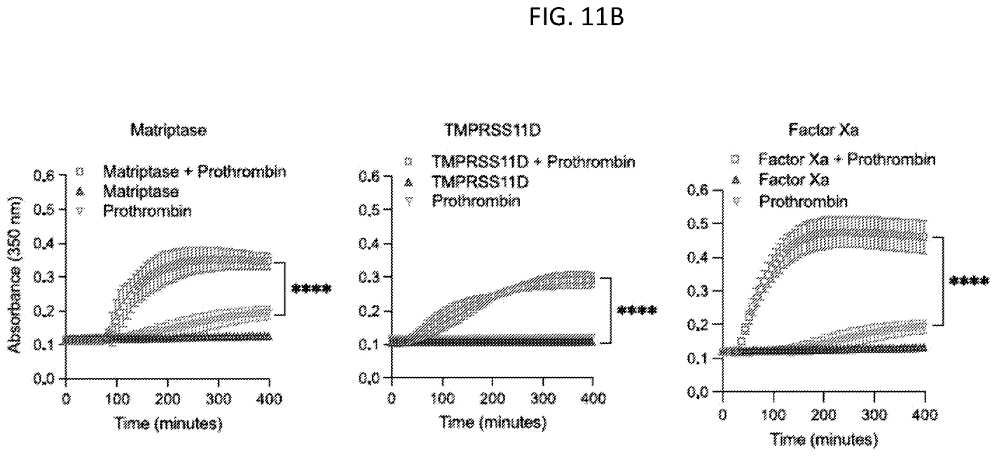

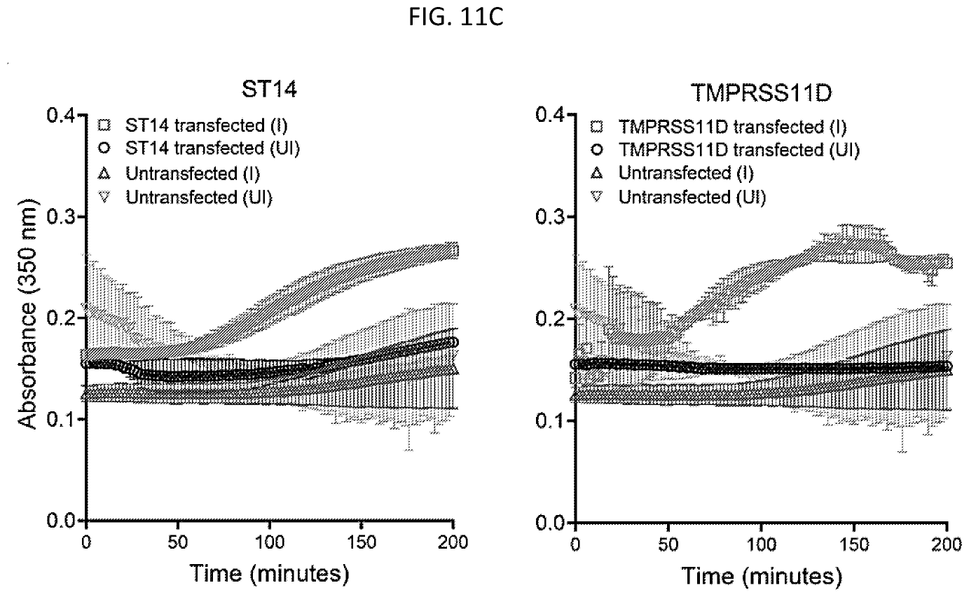

To address if the profibrotic serine protease released by infected NHBE cells is the result of cell surface shedding, various protease inhibitors were added to NHBE cells during pSARS-2 infection. The infected supernatants collected in the presence of the protease inhibitors were assayed for fibrin clot formation. The results showed that the presence of metalloproteinase inhibitors BB-94 or prinomastat during the viral infection significantly reduced fibrin clot formation (FIG. 9E). To confirm that BB-94 did not inhibit fibrin clotting, the experiment was repeated, but with protease inhibitors added post-infection in the fibrin clotting step. The result showed that BB-94 did not inhibit the fibrin clotting step (FIG. 10), suggesting the metalloproteinase inhibitors reduced the infection-induced cell surface shedding of profibrotic enzymes. Consistently, SARS-COV-2 infection of NHBE cells released matriptase into cell culture supernatant (FIG. 9F), suggesting the infection-induced shedding of matriptase. To address if matriptase can activate prothrombin, the cleavage of the fluorogenic prothrombin peptide Thrb-324 by recombinant catalytic matriptase and HAT was examined. Both enzymes cleaved the prothrombin peptide (FIG. 11A). Further, both enzymes promoted fibrin clot formation similar to Factor Xa in component-based fibrin clotting assays by mixing the purified enzymes with prothrombin and fibrinogen (FIG. 11B). To address if the expression of ST14 or TMPRSS11D on cells is sufficient to trigger infection-induced fibrin clotting, non-clotting ACE2-293T cells were transfected with plasmids encoding full length ST14 or TMPRSS11D genes, and infected with a delta variant of pSARS-2 for fibrin clotting assays. The results showed that the infection of either ST14 or TMPRSS11D transfected but not untransfected ACE2-293T cells generated fibrin clots (FIG. 11C). Together, these results show that SARS-COV-2 infection of NHBE cells induced shedding of TMPRSS proteins, such as matriptase and HAT, that are capable of activating prothrombin for fibrin clot formations.

### Example 5

### Infected NHBE Cells Induced Acute COVID BALF to Form Fibrin Clots Ex Vivo

The above work showed that SARS-COV-2 infection of lung epithelial cells resulted in activation and shedding of membrane bound serine proteases, including matriptase and HAT, that are capable of activating prothrombin and inducing fibrosis. As the concentrations of prothrombin and fibrinogen are elevated in acute COVID BALF (FIG. 1), the risk of fibrosis is expected to be higher in the acute samples than in the recovered or healthy BALF samples. It is not clear, however, the contribution of SARS-COV-2 infection to BALF fibrin clot formation and whether the elevated levels prothrombin and fibrinogen are sufficient to form fibrin clots without the viral infection. To address this, both healthy and COVID BALF samples were concentrated 20 fold to approximate lung epithelial lining fluid, and the fibrin clotting assays were performed in the presence of pSARS-2 infected or uninfected NHBE cells.

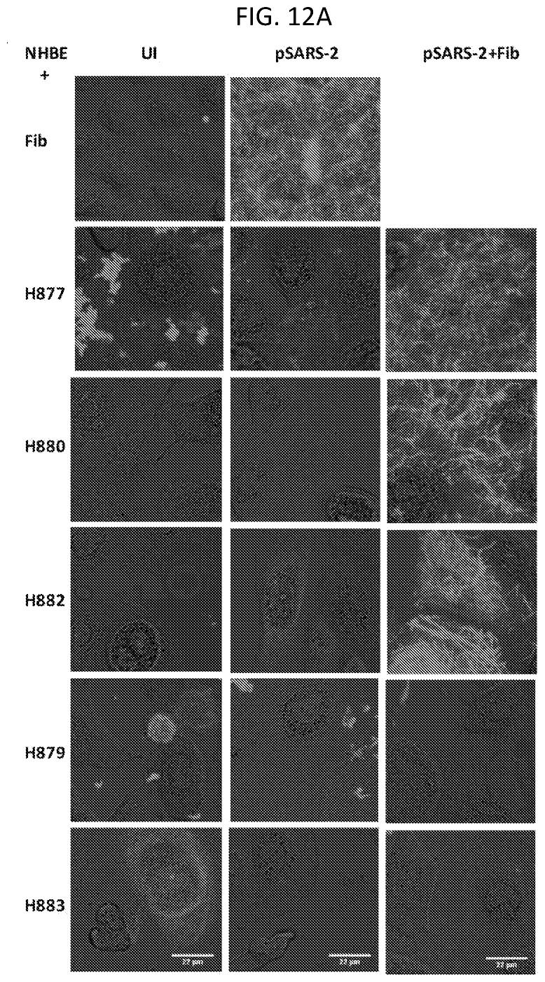

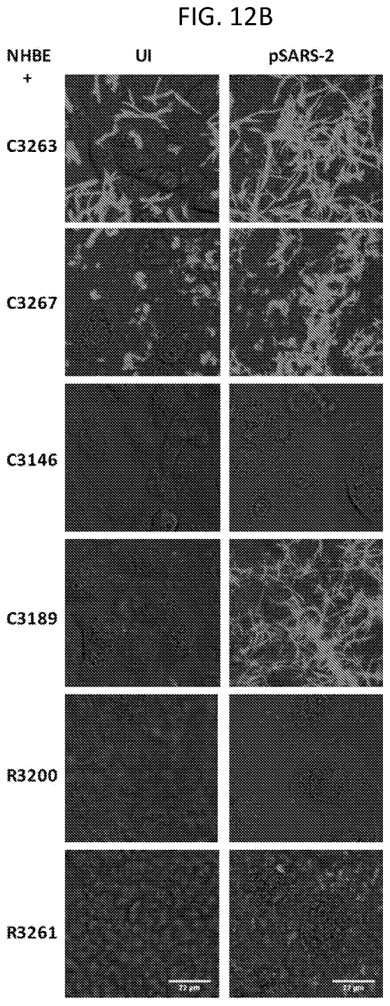

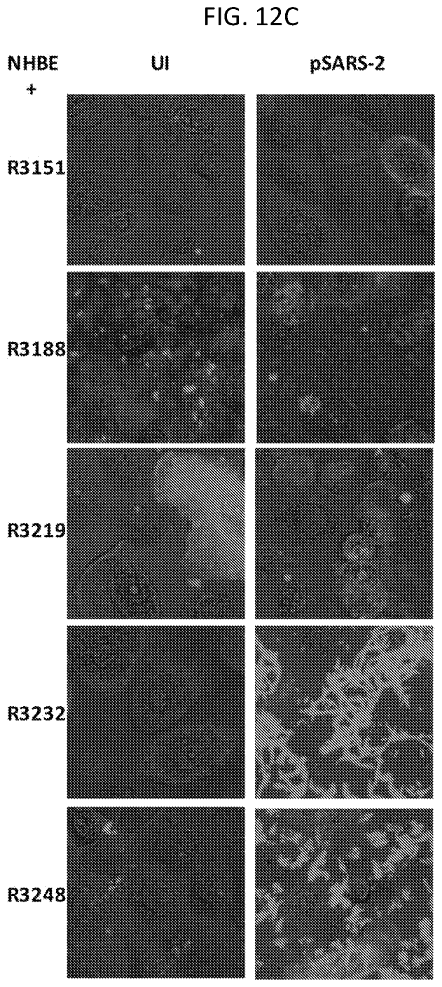

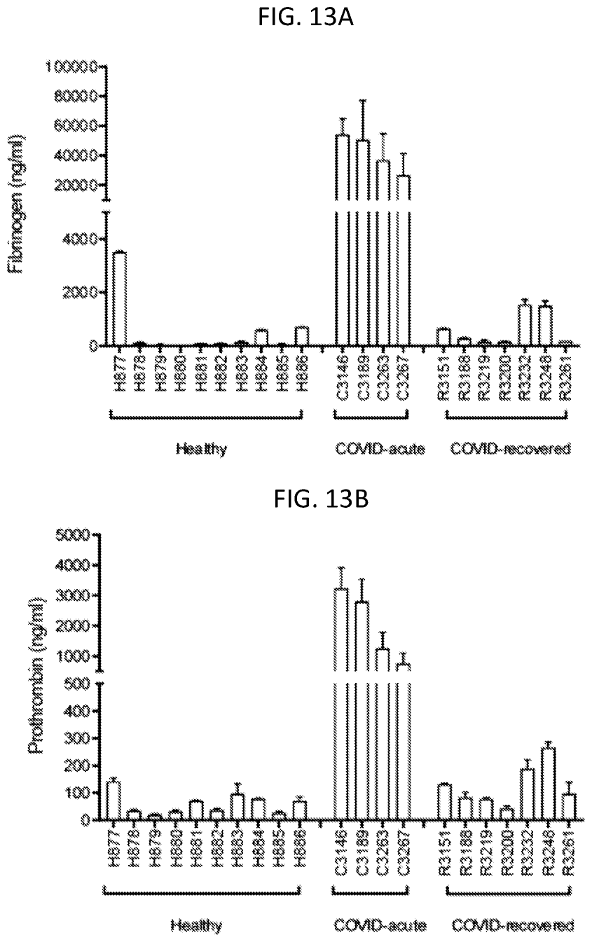

As expected, fibrin clots were readily detected in infected but not uninfected NHBE cells in the presence of fibrinogen (FIG. 12A, top row). In the presence of the healthy BALF samples, no significant fibrin clot formations were detected regardless of the viral infections (FIG. 12A). However, three of the infected NHBE cells induced fibrin clots when exogenous fibrinogen was supplemented into the healthy BALF samples (H877, H880, H882), suggesting the fibrinogen concentrations in the healthy BALF are insufficient to induce fibrin clotting. In contrast to the healthy BALF, three of the acute COVID BALF (C3263, C3267, and C3189) supported fibrin clot formation in the infected NHBE cells without addition of fibrinogen (FIG. 12B). Interestingly, visible fibrin clots were observed in uninfected NHBE cells in the presence of C3263 BALF, suggesting the presence of prior activated thrombin in this BALF sample. In all three acute COVID cases, the viral infection either triggered or enhanced fibrin clot formations, illustrating the viral contribution to BALF fibrin clot formation. Consistent with their lower concentrations of fibrinogen in recovered COVID BALF samples (FIG. 1E, FIG. 13), a majority of the recovered samples did not support fibrin clots with or without the viral infection (FIGS. 12B and 12C). Unlike the healthy BALF tested, fibrin clot formation was visible in COVID-recovered sample, R3232, in the presence but not absence of the viral infection (FIG. 12C), suggesting a potential risk of fibrosis in recovered COVID lungs. Interestingly, the fibrinogen concentration in R3232 was higher than other recovered COVID samples despite being significantly lower than those in acute COVID samples (FIG. 13). These findings showed that SARS-COV-2 infection of lung epithelial cells induced fibrin clot formation in most acutely infected lung fluids.

It is worth noting that not all acute COVID BALF showed equal fibrin clot formation. Despite the presence of elevated level of fibrinogen in C3146 BALF (FIG. 1E, FIG. 13A), no significant fibrin clot was detected in infected NHBE cells (FIG. 12B). Similarly, despite supplementing with exogenous fibrinogen, no fibrin clots were observed in BALF from two of the healthy donors (H879 and H883) (FIG. 12A), suggesting fibrinogen may not be the only factor controlling fibrosis, and there may be other fibrinolytic factors present in BALF to suppress fibrosis. Indeed, anti-coagulation factors, such as plasminogen, antithrombin-III and serine protease inhibitors (SERPIN) were present in both healthy and COVID BALF (Table 1), and the levels of plasminogen and antithrombin-III were also increased in the acute BALF C3146 (FIG. 1E). Together, these data showed that SARS-COV-2 infection of lung epithelial cells induced a cell-mediated fibrin clotting in alveolar fluid that potentially account for acute fibrosis observed in severe COVID cases.

### Example 6

### Preclinical Trial Assessing Safety and Efficacy of Nebulization of Direct Thrombin Inhibitors

This example describes studies to determine the safety doses of nebulizing thrombin inhibitors to respiratory track and alveolar space and to compare the nebulization treatment with IV injection for potential inhibition of airway coagulation. However, one skilled in the art will appreciate that methods that deviate from these specific methods can also be used.

Clinical grade of direct thrombin inhibitors, such as argatroban and dabigatran are delivered to rhesus macaques by aerosol using nebulizer in a dose escalation protocol. Both the drug concentration and delivery frequency will be included as variables in this study. Normal saline with formulation compounds of the direct thrombin inhibitors are used in parallel as controls. Treated monkeys are monitored by blood chemistry (CBC) and for signs of clinical adverse effects. Bronchoalveolar lavage (BAL) is taken regularly and tested for the concentration of thrombin inhibitor drug in BAL, as well as its ability to inhibit fibrin deposition using an in vitro coagulation assay (such as described in Example 5, above).

As comparisons, the same drug or saline control is administered to monkeys by IV injection using recommended (mg/kg) doses for human. Treated monkeys are monitored by blood chemistry and for signs of clinical adverse effects. Bronchoalveolar lavage (BAL) is taken regularly and tested for the concentration of thrombin inhibitor drug in BAL as well as its ability to inhibit fibrin deposition using the in vitro coagulation assay.

It will be apparent that the precise details of the methods or compositions described may be varied or modified without departing from the spirit of the described aspects of the disclosure. We claim all such modifications and variations that fall within the scope and spirit of the claims below.

1. A method of treating or inhibiting viral infection-induced airway fibrosis in a subject, comprising administering to the subject an effective amount of a composition comprising one or more direct thrombin inhibitors.

2, The method of claim 1, wherein the viral infection-induced airway fibrosis is a coronavirus infection-induced airway fibrosis.

3. The method of claim 2, wherein the coronavirus is severe acute respiratory syndrome (SARS)-CoV-2.

4. The method of claim 1, wherein the administering is by inhalation.

5. The method of claim 4, wherein the composition is administered as an aerosol.

6. The method of claim 5, wherein the composition is administered using a nebulizer, a dry powder inhaler, or a metered dose inhaler.

7. The method of claim 1, wherein the composition further comprises a pharmaceutically acceptable carrier.

8. The method of claim 1, wherein the direct thrombin inhibitor is selected from the group consisting of argatroban, dabigatran, ximelagatran, hirudin, lepirudin, desirudin, and bivalirudin.

9. The method of claim 8, wherein the direct thrombin inhibitor is argatroban or dabigatran.

10. The method of claim 9, wherein the dose of the argatroban or dabigatran is about 0.1 μg/kg to about 10 mg/kg.

11. A method of treating or inhibiting viral infection-induced airway fibrosis in a subject, comprising administering to the subject an effective amount of a composition comprising one or more serine protease inhibitors or metalloprotease inhibitors.

12. The method of claim 11, wherein the viral infection-induced airway fibrosis is a coronavirus infection-induced airway fibrosis.

13. The method of claim 12, wherein the coronavirus is severe acute respiratory syndrome (SARS)-CoV-2.

14. The method of claim 11, wherein the administering is by inhalation.

15. The method of claim 14, wherein the composition is administered as an aerosol.

16. The method of claim 15, wherein the composition is administered using a nebulizer, a dry powder inhaler, or a metered dose inhaler.

17. The method of claim 11, wherein the composition further comprises a pharmaceutically acceptable carrier.

18. The method of claim 11, wherein the serine protease inhibitor is camostat or nafamostat or the metalloprotease inhibitor is batimastat (BB-94) or prinomastat.

19. The method of claim 1, further comprising administering to the subject an additional treatment for the viral infection.

20. The method of claim 19, wherein the additional treatment comprises one or more of an antiviral compound, a corticosteroid, and a monoclonal antibody.

21. The method of claim 20, wherein the subject has a SARS-COV-2 infection and the antiviral compound is one or more of nirmatrelvir, ritonavir, remdesivir, and molnupiravir.

22. The method of claim 20, wherein the subject has a SARS-COV-2 infection and the monoclonal antibody is bebtelovimab.


SEQUENCE LISTING (1 sequence)

SEQ ID NO: 1 - protein, 10 aa, synthetic construct
FNPRTFGSGE